(19) United States
(12) Patent Application Publication
(10) Pub. No.: US 2024/0287489 A1
(43) Pub. Date: Aug. 29, 2024

(54) CHITINOLYTIC ENZYME-BASED PLANT PROTECTION AGENTS

(71) Applicants: GLOBACHEM NV, Sint-Truiden (BE); FRAUNHOFER-GESELLSCHAFT ZUR FÖRDERUNG DER ANGEWANDTEN FORSCHUNG E.V., München (DE)

(72) Inventors: Guy VAN DAELE, Westerlo (BE); Liesbeth VOGELS, Kuringen (BE); Liesbeth ZWARTS, Pellenberg (BE); Christel VAN CEULEBROECK, Neerlanden (BE); Siham AGDOUR, Stolberg (DE); Christian SCHMITZ, Monschau (DE); Stefan RASCHE, Erkelenz (DE)

(73) Assignees: GLOBACHEM NV, Sint-Truiden (BE); FRAUNHOFER-GESELLSCHAFT ZUR FÖRDERUNG DER ANGEWANDTEN FORSCHUNG E.V., München (DE)

(21) Appl. No.: 18/570,470

(22) PCT Filed: Jun. 15, 2022
(86) PCT No.: PCT/EP2022/066368

§ 371 Date: Dec. 14, 2023

(30) Foreign Application Priority Data
Jun. 15, 2021 (EP) 21 179 559.6

(51) Int. Cl. C12N 9/42; A01N 63/50; A01P 3/00; A01P 7/04

(52) U.S. Cl. CPC C12N 9/2442; A01N 63/50; A01P 3/00; A01P 7/04; C12Y 302/01014

(57) ABSTRACT

The present invention relates to chitinolytic enzymes, and in particular for a use thereof in plant protection. The invention also relates to a nucleic acid encoding a chitinolytic enzyme a method for producing a chitinolytic enzyme, a plant comprising a chitinolytic enzyme or a nucleic acid encoding the same, a composition of at least one chitinolytic enzyme. The invention relates in particular to the use of a chitinolytic enzyme or a composition comprising the same as a plant protection agent and to a method of protecting plants from pests.

### CROSS REFERENCE TO RELATED APPLICATIONS

The present application is a U.S. National Phase Application under 35 U.S.C. § 371 of International Patent Application No. PCT/EP2022/066368 filed Jun. 15, 2022, which claims priority of European Patent Application No. 21 179 559.6 filed Jun. 15, 2021. The entire contents of which are hereby incorporated by reference.

### FIELD OF INVENTION

The present invention relates to chitinolytic enzymes, and in particular for a use thereof in plant protection. The invention also relates to a nucleic acid encoding a chitinolytic enzyme a method for producing a chitinolytic enzyme, a plant comprising a chitinolytic enzyme or a nucleic acid encoding the same, a composition of at least one chitinolytic enzyme. The invention relates in particular to the use of a chitinolytic enzyme or a composition comprising the same as a plant protection agent and to a method of protecting plants from pests.

### BACKGROUND

Chitin is a polymer of N-acetylglucosamine, a derivative of the saccharide glucose, with the chemical formula (C₈H₁₃O₅N)ₙ. The long-chain polysaccharide occurs in a wide variety of different organisms across different clades. For example, chitin is a primary component of cell walls of fungi, the exoskeletons of arthropods, such as crustaceans and insects, the radulae of mollusks, and the scales of fish. Chitin has a structure that is comparable to cellulose.

The biological conversion of chitin polysaccharides into shorter oligomers requires hydrolytic enzymes that contain conserved chitin-binding domains and chitin-specific active sites. Many chitinolytic enzymes are produced by a variety of bacteria and fungi for degradation of chitin as energy source. All of them are glycosyl hydrolases, but they differ in terms of reaction mechanism, thermostability and product characteristics [Patil et al., Enzyme Microb. Technol., 2000. 26: p. 473-483]. Chitinolytic hydrolases can be categorized according to their mode of action. Endo-chitinases (EC 3.2.1.14) bind randomly to a chitin polysaccharide strand and hydrolyze internal glycosidic bonds producing various fragment sizes ranging from dimers to polymers. In contrast, exo-chitinases (EC 3.2.1.29) bind to the reducing or non-reducing end of chitin and release monomeric and to lesser extent dimeric GlcNAc units. These enzymes are necessary for the complete degradation of chitin. Finally, chitobiases (EC 3.2.1.29) cleave GlcNAc dimers to release GlcNAc monomers [Tews et al., Nat. Struct. Biol., 1996. 3: p. 638-648]. Other enzymes such as cellulase and lysozyme are also known to exhibit some hydrolytic activity towards chitin but are not specific for these substrates [Wu et al., J Food Sci Technol, 2012. 49(6): p. 695-703; Aiba, Carbohydr Res, 1994. 261: p. 297-306].

Common pests of plants include fungi, insects and mollusks. Pest infestation can result in crop loss and contamination of agricultural products with undesired side products.

Current commercially applied chemical pesticides comprise substances of the groups of organochlorines, organophosphates, carbamates, pyrethroids, triazines and neonicotinoids for use as insecticides, herbicides, fungicides and rodenticides. These pesticides are used not only for agricultural areas, but also for non-agricultural public urban green areas, sports fields, pet shampoos, building materials or boat bottoms to eliminate or prevent the presence of unwanted species. These substances have been critically reviewed as numerous negative health effects have been associated with chemical pesticides and high occupational, intentional or accidental exposure can result in hospitalization or death, whereas exposure occurs via skin contact, ingestion of contaminated consumables or inhalation upon which they may be metabolized, excreted, stored or accumulated in the body fat. [Nicolopoulou-Stamati et al., Front. Public Health, 2016, 4:148]

An ideal pesticide should not only be non-hazardous to the human health, but should also be environmentally friendly and as efficient and specific as possible for protecting a plant from a given pest. Moreover, a pesticide should ideally avoid development of a resistance in pests. There is still a need for new products that fulfil these criteria.

### DESCRIPTION OF INVENTION

The present invention aims to overcome the issues of current plant pesticides by providing an enzyme-based approach on pest control. Specifically, the inventive approach relies on chitinolytic enzymes for protecting plants from pests. The enzymes specifically degrade chitin, which is not produced in humans or other higher animals, and are thus expected to not pose a risk for human consumption or for other non-target organisms. Moreover, the enzymes are fully biologically degradable and thus environmentally friendly. Apart from this, given that chitin is a central structural component in pests such as fungi or insects, as well as in the radula of mollusks, such pests are not expected to easily develop a resistance against chitinolytic enzymes.

The present inventors have found out that chitinolytic enzymes can indeed be used for protecting plants from pests such as fungi and insects. Moreover, the inventors have found novel chitinolytic enzymes, which allow an improved enzymatic degradation of crystalline chitin. Chitinolytic enzymatic processes thus provided are competitive to established chemical methods of chitinolysis. The inventors further developed constructs suitable for industrial production by establishing suitable terminal tags for secretion of the enzyme and for purification, as well as an appropriate purification process.

The present application shows for the first time how a chitinolytic enzyme is applied as plant protection agent to counter pest infestation, e.g. via application of the chitinolytic enzymes to a surface of a plant or a part thereof.

Further, the invention makes an important contribution to the prior art pesticides, as the chitinolytic enzymes and their ensuing use as plant protection agent offers distinct advantages when compared to established substances. Such advantages may include the absence of safety hazards during handling or absence of pathogenicity upon entry into the food chain.

Accordingly, the present invention provides the following preferred embodiments:
[1] A chitinolytic enzyme comprising a first amino acid sequence that is at least 70%, such as 100%, identical to an amino acid sequence selected from the group of SEQ ID NOs: 1-5.
[2] The chitinolytic enzyme according to [1], wherein the chitinolytic enzyme further comprises a second amino acid sequence fused to the N-terminus of the first amino acid sequence.
[3] The chitinolytic enzyme according to [2], wherein the second amino acid sequence is less than 50 amino acids in length.
[4] The chitinolytic enzyme according to [2] or [3], wherein the second amino acid sequence consists of a signal peptide, preferably a PelB leader sequence (SEQ ID NO: 6).
[5] The chitinolytic enzyme according to any one of [1]-[4], wherein the chitinolytic enzyme further comprises a third amino acid sequence fused to the C-terminus of the first amino acid sequence.
[6] The chitinolytic enzyme according to [5], wherein the third amino acid sequence is less than 50 amino acids in length.
[7] The chitinolytic enzyme according to [5] or [6], wherein the third amino acid sequence consists of a purification tag, preferably a 6×His tag (SEQ ID NO: 7).
[8] The chitinolytic enzyme according to any one of [2]-[7], wherein the enzyme comprises or consists of a first, a second and a third amino acid sequence.
[9] The chitinolytic enzyme according to any one of [1]-[8], comprising the amino acid sequence according to SEQ ID NO: 1.
[10] The chitinolytic enzyme according to any one of [1]-[8], comprising the amino acid sequence according to SEQ ID NO: 2.
[11] The chitinolytic enzyme according to any one of [1]-[8], comprising the amino acid sequence according to SEQ ID NO: 3.
[12] The chitinolytic enzyme according to any one of [1]-[8], comprising the amino acid sequence according to SEQ ID NO: 4.
[13]. The chitinolytic enzyme according to any one of [1]-[8], comprising the amino acid sequence according to SEQ ID NO: 5.
[14]. The chitinolytic enzyme according to any one of [1]-[13], essentially consisting of a first amino acid sequence that is at least 70%, such as 100%, identical to an amino acid sequence selected from the group of SEQ ID NOs: 1-5.
[15] The chitinolytic enzyme according to any one of [1] or [9]-[14], consisting of a first amino acid sequence that is at least 70%, such as 100%, identical to an amino acid sequence selected from the group of SEQ ID NOs: 1-5.
[16] A nucleic acid encoding the chitinolytic enzyme according to any one of [1]-[15].
[17] A vector comprising the nucleic acid according to [16].
[18]. The vector according to [17], which is an expression vector.
[19] A host cell comprising the nucleic acid according to or the vector according to or [18].
[20] The host cell according to [19], which is a plant cell or a microbial cell.
[21] The host cell according to [20], wherein the plant is an arable crop, fruit-bearing plant or vegetable.
[22] The host cell according to [20], wherein the microbial cell is a bacterial cell.
[23] The host cell according to [22], wherein the bacterium is E. coli.
[24] A plant comprising the chitinolytic enzyme according to any one of [1]-[15], or the nucleic acid according to [16, or the vector according to or [18].
[25] A method for producing a chitinolytic enzyme according to any one of [1]-[15], the method comprising culturing a host cell according to any one of [19-[23].
[26] The method according to [25], further comprising harvesting the cells and/or the supernatant during and/or after the culture, preferably harvesting the supernatant after the culture.
[27] The method according to or [26], further comprising purifying the chitinolytic enzyme.
[28] A composition comprising at least one chitinolytic enzyme according to any one of [1]-[15].
[29] The composition according to [28], wherein the composition comprises at least two different chitinolytic enzymes according to any one of [1]-[15].
[30] The composition according to [29], wherein the at least two different chitinolytic enzymes exert a synergistic effect.
[31] The composition according to [30], wherein the synergistic effect is characterized by a disproportionally improved chitin degradation rate (compared to the individual enzymes).
[32] The composition according to any one of [28]-[31], comprising a chitinolytic enzyme comprising or (essentially) consisting of SEQ ID NO: 1 and a chitinolytic enzyme comprising or (essentially) consisting of SEQ ID NO: 2.
[33] The composition according to any one of [32], further comprising a chitinolytic enzyme comprising or (essentially) consisting of SEQ ID NO: 3.
[34] The composition according to any one of [28]-[33] that is a plant protection agent.
[35] Use of a composition comprising at least one chitinolytic enzyme as a plant protection agent.
[35] Use of the composition according [35], wherein the composition is according to any one of [28]-[35].
[37] Use of the composition according to or as a plant protection agent against an organism that contains chitin.
[38] Use of the composition according to any one of [35]-[37], wherein the plant protection agent is against a fungus and/or against an insect.
[39] Use of a composition according to any one of [35]-[38], wherein the fungus is a Fusiarum or Septoria species.
[40] Use of the composition according to or as a plant protection agent against abiotic stress, wherein the abiotic stress preferably is drought, frost or flooding stress.
[41] Use of the composition according to or as a biostimulant against abiotic stress in plants, wherein the abiotic stress preferably is drought, frost or flooding stress.
[42] Use according to any one of [35]-[41], wherein the plant is an arable crop, fruit-bearing plant or vegetable.
[43] A method of protecting a plant from pests and/or abiotic stress, the method comprising the application of a composition comprising at least one chitinolytic enzyme on the plant or a part thereof.
[44] The method according to [43], wherein the composition is according to any one of [28]-[35].
[45] The method according to anyone of or [44], wherein the plant or plant part is soaked in the composition.
[46] The method according to anyone of [43]-[45], wherein the composition is applied by spraying to a surface of the plant or part of the plant, such as a leave or seed.
[47] The method according to any one of [43]-[46], wherein the pest is an organism that contains chitin.
[48] The method according to any one of [43]-[47], wherein the pest is a fungus or an insect.
[49] The method according to [48], wherein the fungus is a Fusiarum or Septoria species.
[50] The method according to any one of [43]-[49], wherein the plant is an arable crop, fruit-bearing plant or vegetable.
[51] The method according to any one of [43]-[50], wherein the abiotic stress is drought, frost or flooding stress.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1C. Analysis of P. orarium supernatant for the hydrolysis of chitin. FIG. 1A: Quantification of chitinolytic activity of culture supernatant (SN) from P. orarium after incubation with chitin powder as a substrate for 16 h at 30° C. E. coli BL21 wild-type culture supernatant was used as a negative control (NC). Reducing sugars were quantified using a reducing end assay and a calibration curve of N-acetyl glucosamine was prepared freshly. The reducing end assays were carried out in three replicates (n=3). FIG. 1B: Zymogram of freshly prepared supernatant of a P. orarium culture grown on chitin as the sole carbon source. For the identification of active chitinolytic enzymes, 10% (v/v) glycol chitin was incorporated in the gel and fluorescent dye was applied after incubation of the gel at 30° C. for 2 h. E. coli BL21 supernatant was used as a negative control (NC). FIG. 1C: Fresh P. orarium cultures supernatant was incubated with 5% (w/v) chitin powder at 30° C. for 16 h. After heat inactivation (95° C., 10 min) 0.3 μl of the hydrolysate was separated by TLC. E. coli BL21 supernatant was used as a negative control (NC) and commercial chitin standard molecules (monomer-hexamer) were used as a size marker. kDa=kilo Dalton; DP=degree of polymerization.

FIG. 2. Construct variants for each putative chitinase gene cloned in E. coli DH5a using the golden gate cloning technology. Variants comprising the PelB signal peptide and 6×His tag (in red frame) were selected for upscaled expression and purification.

FIG. 3. Spider plot summarizing the specific activity of all chitinase variants generated, expressed as nmol reducing ends/mg enzyme. Cell lysates and culture supernatant were assessed separately for each variant. Enzyme activity was quantified using the reducing end assay and enzyme quantities were determined densitometrically after immunoblot analysis. PelB: PelB signal peptide; cyto: cytosolic expression; dsbA: dsbA fusion protein; NL: natural signal peptide; His: 6×His tag; T54: Tag54/6×His combi tag; P: cell pellet lysate; SN: culture supernatant.

FIG. 4. Coomassie R-250 stained SDS-PAGE gel (left panel) and corresponding immunoblot (6×His tag detection; right panel) of the elution fractions of chitinases 1-5 (C1-C5) after IMAC purification. For the Coomassie stained gel, 8 μl pf pure sample were loaded. For immunoblot analysis, samples for C1 was pre-diluted 1:5 with PBS (pH 7.4) and 4 μl were loaded for each sample. kDa=kilo dalton; M=protein marker. The results shown are representative of at least three replicates.

FIG. 5A-5B. Temperature optima and stability of recombinant chitinases (C1-C5) produced in E. coli BL21. Activity was determined using a reducing end assay with chitin powder as substrate. Highest measured absolute activity was taken as 100%. FIG. 5A: Activity within the temperature range (10-60° C.) at pH 8. FIG. 5B: Temperature stability at 60° C. Residual enzyme activity was determined at standard conditions after pre-incubation of enzymes for 0-240 min. ⋅: C1, ▪: C2, ▴: C3, ▾: C4, ♦: C5. Data represents means of three replicate experiments (n=3).

FIGS. 6A-6B. Optimum pH and salinity of recombinant chitinases (C1-C5) produced in E. coli BL21. Activity was determined using a reducing end assay with chitin powder as substrate. Highest measured absolute activity was taken as 100%. FIG. 6A: Effect of different pH on enzymatic activity (pH 4-11) at 30° C. FIG. 6B: Activity within different NaCl contents (0-20% w/v) at pH 8, 30° C. ⋅: C1, ▪: C2, ▴: C3, ▾: C4, ♦: C5. Data represents means of three replicate experiments (n=3).

FIG. 7. Michaelis-Menten fitted line plot of recombinant chitinases (C1-C5) (GraphPad software). The inserted Lineweaver-Burke plot relates reaction velocities to released GlcNAc associated with the concentration of chitin powder (0-150 mg/ml). Vmax and Km were calculated from the non-linear fit model. Data represents means of three replicate experiments (n=3).

FIG. 8. Thin layer chromatogram of enzymatic hydrolysis products from recombinant chitinases 1-5 (C1-C5) incubated for 16 h at 30° C. with chitin powder, chitosan or colloidal chitin. Chitin and chitosan standard molecules ranging from degree of polymerization (DP) 1 up to DP 5 were used to identify hydrolysis products. The results shown are representative of three replicates.

FIG. 9. Thin layer chromatograms of enzymatic hydrolysis products from recombinant chitinases (C1-5) incubated for 16 h with chitin oligomers of degree of polymerization 2-6 (DP2-DP6) at a concentration of 4 mg/ml. Spots on the far right are untreated standard molecules. The results shown are representative of three replicates.

FIG. 10. Principle of the golden gate cloning technology. Type IIs restriction enzymes such as BsaI cut outside of their recognition site (GAGACC) resulting in single stranded overhangs and the loss of the recognition site itself. The overhangs (N) are specifically designed in silico to allow a directed ligation of the gene fragments and the vector backbone. The order of the individual genes is thus predetermined and cloning efficiency is increased. Digestion and ligation can be carried out in one reaction vessel.

FIG. 11. Schematic overview of an exemplary cascading multi-enzyme process using chitin as insoluble substrate. Solid substrate residues (SR) and products (P) may serve as additional substrates (S) for sequential reactions by different enzymes (E). Thereby, different final products could be obtained. Enzymatic feed-back reactions further increasing complexity are not considered in this scheme.

FIG. 12. Thin layer chromatogram of hydrolysis products from mixture DoE of recombinant chitinases 1-5 with chitin as a substrate. A total sample volume of 0.1 μl was loaded by sequential application of 0.1 μl. A mixture of chitin oligomers was used a size marker to identify products. Differences were identified regarding the total product yields reflected also by the quantitative data from the reducing end assay. M=chitin size marker; DP=degree of polymerization.

FIG. 13. Exemplary LC-MS spectra of hydrolysis sample from chitinase mixture design (Run 15 and 12). Oligomeric chitin products were identified by their respective molecular weights. Peak integration was used to determine average mixture compositions. DP=degree of polymerization. Mono-d: mono-deacetylated product.

FIG. 14. Response plot (nmol/ml×h) for tested optimized solutions from the chitinase mixture design. Three solutions were tested to maximize the conversion rate of chitin to chitin oligomers (Black dots) and three solutions for minimized conversion rate (grey dots). The minimized solutions were used to further validate the predictability of the design model. The total molar enzyme concentration was set to 2 μM. Response plots highlight which factor combinations are necessary to achieve the desired output (red color=high response; blue color=low response). A: chitinase 1; B: chitinase 2; C: chitinase 3.

FIG. 15. Thin layer chromatogram of oligomeric chitin products for tested solutions for minimized (S1-S3) and maximized (S1-S3) chitin conversion rates. A total sample volume of 0.3 μl were loaded sequentially in 0.1 μl spots. Samples for S1-S3 Max were diluted 1:3 with dH20 before application. Oligomeric chitin standards were used as a size marker. DP=degree of polymerization; M-Chi: size marker chitin oligomers; M-COS: size marker chitosan oligomers.)

FIG. 16. Exemplary LC-MS spectra from two optimized solutions for maximized (S1 Max) and minimized (S1 Min) chitin conversion rates. Oligomeric chitin products were identified by their respective molecular weights. Peak integration was used to determine average mixture compositions. DP=degree of polymerization.

FIGS. 17A-17G. Use of chitinases as plant protection agent. F. culmorum growth and plant health was assessed in the presence of chitinases. FIG. 17A Control; FIG. 17B Control+F. culmorum; FIG. 17C Chitinase 1+F. culmorum; FIG. 17D Chitinase 2+F. culmorum; FIG. 17E Chitinase 3+F. culmorum; FIG. 17F Chitinase 4+F. culmorum; FIG. 17G Chitinase 5+F. culmorum.

FIGS. 18A-18E. Use of chitinase 1 as plant protection agent against abiotic stresses. Plant length (PL) FIG. 18A: after foliar application on corn in presence of drought or flooding stress; FIG. 18B: after foliar, seed or foliar+seed application on corn in presence of drought stress; FIG. 18C: after foliar application on rice in presence of salt stress; FIG. 18D: after foliar application on corn in presence of salt stress; FIG. 18E: after seed application on barley in presence of drought stress.

FIGS. 19A-19B. Use of chitinase 1 as plant protection agent against abiotic stresses. Yield FIG. 19A: of pears after foliar application in presence of frost stress; FIG. 19B: of cherries after foliar application in presence of frost stress.

### DETAILED DESCRIPTION OF INVENTION

Unless specifically defined herein, all technical and scientific terms used herein have the same meaning as commonly understood by a skilled artisan in the fields of enzymology, plant protection, biochemistry, genetics, and molecular biology.

All methods and materials similar or equivalent to those described herein can be used in the practice or testing of the present invention, with suitable methods and materials being described herein.

The term “about” when used in the context of the present invention means that the value following the term “about” may vary within the range of +/−20%, preferably in the range of +/−15%, more preferably in the range of +/−10%.

All publications, patents and patent applications cited herein are hereby incorporated by reference in their entirety for all purposes. In case of conflict, the present specification, including definitions, will prevail over the cited references. Further, the materials, methods, and examples are illustrative only and are not intended to be limiting, unless otherwise specified.

As used herein, each occurrence of terms such as “comprising” or “comprises” may optionally be substituted with “consisting of” or “consists of”. The term “essentially consists of” in the context of compounds or compositions means that specific further components can be present that do not materially affect the essential characteristics of the compound or composition. For example, a chitinolytic enzyme essentially consisting of a certain amino acid sequence can consist of said amino acid sequence and additional N- and/or C-terminal sequences that do not materially affect the chitinolytic activity of the enzyme (such as a second and/or third sequence as defined herein).

The present invention aims to overcome the issues of current plant pesticides by providing an enzyme-based approach on pest control. Specifically, the inventive approach relies on chitinolytic enzymes for protecting plants from pests. These enzymes specifically degrade chitin, which is not produced in humans or other higher animals and are thus expected to not pose a risk for human consumption or for other non-target organisms. In the present application, the term “chitinolytic enzyme” is used synonymously with the term “chitinase”.

Moreover, the inventors have found and characterized novel chitinolytic enzymes from a newly identified species of Photobacterium, which exhibit advantageous properties compared to prior art chitinolytic enzymes and which e.g. allow a vastly improved enzymatic turnover. The inventors surprisingly found that combinations of chitinolytic enzymes can achieve synergistically improved chitin degradation rates.

Chitinolytic processes thus provided are competitive to established chemical methods of chitinolysis. The present disclosure is described in more detail as follows.

### Chitinolytic Enzymes

The present invention relates to a chitinolytic enzyme, a composition comprising one or more chitinolytic enzymes, as well as to a plant protection agent, such as a pesticide, comprising or (essentially) consisting of one or more chitinolytic enzymes.

A chitinolytic enzyme as disclosed herein can comprise a first amino acid sequence that is at least 70% identical (such as 100% identical) to an amino acid sequence selected from the group of SEQ ID NOs: 1-5. For example, the first amino acid sequence is at least 80%, at least 90%, at least 95%, at least 98% or at least 99% identical to an amino acid sequence selected from the group of SEQ ID NOs: 1-5. Preferably, the first amino acid sequence is at least 98%, or at least 99% identical, and most preferably 100% identical to an amino acid sequence selected from the group of SEQ ID NOs: 1-5. Thus, the enzyme can comprise a first amino acid sequence selected from the group of SEQ ID NOs: 1-5.

In line with this, a preferred chitinolytic enzyme can also essentially consist of a first amino acid sequence that is at least 70% identical (such as 100% identical) to an amino acid sequence selected from the group of SEQ ID NOs: 1-5. For example, the first amino acid sequence is at least 80%, at least 90%, at least 95%, at least 98% or at least 99% identical to an amino acid sequence selected from the group of SEQ ID NOs: 1-5. Preferably, the first amino acid sequence is at least 98%, or at least 99% identical, and most preferably 100% identical to an amino acid sequence selected from the group of SEQ ID NOs: 1-5. Thus, the enzyme can essentially consist of a first amino acid sequence selected from the group of SEQ ID NOs: 1-5.

Accordingly, a chitinolytic enzyme can also consist of a first amino acid sequence that is at least 70% identical (such as 100% identical) to an amino acid sequence selected from the group of SEQ ID NOs: 1-5. For example, the first amino acid sequence is at least 80%, at least 90%, at least 95%, at least 98% or at least 99% identical to an amino acid sequence selected from the group of SEQ ID NOs: 1-5. Preferably, the first amino acid sequence is at least 98%, or at least 99% identical, and most preferably 100% identical, to an amino acid sequence selected from the group of SEQ ID NOs: 1-5. Thus, the enzyme can consist of a first amino acid sequence selected from the group of SEQ ID NOs: 1-5.

For example, the first amino acid sequence can be at least 70% identical to SEQ ID NO: 1.

For example, the first amino acid sequence can be at least 70% identical to SEQ ID NO: 2.

For example, the first amino acid sequence can be at least 70% identical to SEQ ID NO: 3.

For example, the first amino acid sequence can be at least 70% identical to SEQ ID NO: 4.

For example, the first amino acid sequence can be at least 70% identical to SEQ ID NO: 5.

A chitinolytic enzyme as disclosed herein can comprise a first amino acid sequence that exhibits up to 15 amino acid differences (such as no amino acid differences) to an amino acid sequence selected from the group of SEQ ID NOs: 1-5. For example, the first amino acid sequence exhibits up to 10, up to 5, or up to 3, 2 or 1 amino acid differences to an amino acid sequence selected from the group of SEQ ID NOs: 1-5. Preferably, the first amino acid sequence exhibits up to 3, 2 or 1 amino acid differences, most preferably no amino acid differences, to an amino acid sequence selected from the group of SEQ ID NOs: 1-5. Thus, the enzyme can comprise a first amino acid sequence selected from the group of SEQ ID NOs: 1-5.

In line with this, a chitinolytic enzyme as disclosed herein can essentially consist of a first amino acid sequence that exhibits up to 15 amino acid differences (such as no amino acid differences) to an amino acid sequence selected from the group of SEQ ID NOs: 1-5. For example, the first amino acid sequence exhibits up to 10, up to 5, or up to 3, 2 or 1 amino acid differences to an amino acid sequence selected from the group of SEQ ID NOs: 1-5. Preferably, the first amino acid sequence exhibits up to 3, 2 or 1 amino acid differences, most preferably no amino acid differences, to an amino acid sequence selected from the group of SEQ ID NOs: 1-5. Thus, the enzyme can essentially consist of a first amino acid sequence selected from the group of SEQ ID NOS: 1-5.

Accordingly, a chitinolytic enzyme as disclosed herein can consist of a first amino acid sequence that exhibits up to 15 amino acid differences (such as no amino acid differences) to an amino acid sequence selected from the group of SEQ ID NOs: 1-5. For example, the first amino acid sequence exhibits up to 10, up to 5, or up to 3, 2 or 1 amino acid differences to an amino acid sequence selected from the group of SEQ ID NOs: 1-5. Preferably, the first amino acid sequence exhibits up to 3, 2 or 1 amino acid differences, most preferably no amino acid differences, to an amino acid sequence selected from the group of SEQ ID NOs: 1-5. Thus, the enzyme can consist of a first amino acid sequence selected from the group of SEQ ID NOs: 1-5.

For example, the first amino acid sequence can exhibit up to 15 amino acid differences to SEQ ID NO: 1.

For example, the first amino acid sequence can exhibit up to 15 amino acid differences to SEQ ID NO: 2.

For example, the first amino acid sequence can exhibit up to 15 amino acid differences to SEQ ID NO: 3.

For example, the first amino acid sequence can exhibit up to 15 amino acid differences to SEQ ID NO: 4.

For example, the first amino acid sequence can exhibit up to 15 amino acid differences to SEQ ID NO: 5.

When the first amino acid sequence is less than 100% identical and/or has amino acid differences to an amino acid sequence selected from the group of SEQ ID NOs: 1-5, the chitinolytic enzyme preferably has the same or a better degradation rate as a corresponding chitinolytic enzyme (essentially) consisting of any one of SEQ ID NOs: 1-5. For example, when the first amino acid is at least 70% (and less than 100%) identical to the amino acid sequence of SEQ ID NO: 1, the chitinolytic enzyme preferably has the same or a better chitin degradation rate as a chitinolytic enzyme (essentially) consisting of SEQ ID NO: 1. Likewise, for example, when the first amino acid has up to 15 (and at least 1) amino acid differences to the amino acid sequence of SEQ ID NO: 1, the chitinolytic enzyme preferably has the same or a better chitin degradation rate as a chitinolytic enzyme (essentially) consisting of SEQ ID NO: 1. The same applies to SEQ ID NOs: 2, 3, 4 or 5 mutatis mutandis.

When the first amino acid sequence is less than 100% identical and/or has amino acid differences to an amino acid sequence selected from the group of SEQ ID NOs: 1-5, the skilled person knows how to modify the original sequence in order to maintain or improve the chitin degradation rate compared to the reference, i.e. unmodified, sequence (essentially) consisting of an amino acid sequence selected from the group of SEQ ID NOs: 1-5. The chitin degradation rate can be determined using e.g. chitin powder as substrate, for example by a method as described in the examples. Typically, the same method is used to determine the chitin degradation rate of a modified enzyme and that of the reference sequence.

The percentage of “sequence identity” or “% identical” between a first amino acid sequence and a second amino acid sequence may be calculated by dividing [the number of amino acid residues in the first amino acid sequence that are identical to the amino acid residues at the corresponding positions in the second amino acid sequence] by [the total number of amino acid residues in the first amino acid sequence] and multiplying by [100%], in which each deletion, insertion, substitution or addition of an amino acid residue in the second amino acid sequence-compared to the first amino acid sequence—is considered as a difference at a single amino acid residue (i.e. at a single position). The same applies mutatis mutandis to nucleotide sequences.

An “amino acid difference” as used herein can be an amino acid insertion, deletion or substitution, and is preferably a substitution. An amino acid substitution is preferably a conservative substitution as known in the art. Such a conservative substitution can be a substitution in which one amino acid within the following groups (a)-(e) is substituted by another amino acid residue within the same group: (a) small aliphatic, nonpolar or slightly polar residues: Ala, Ser, Thr, Pro and Gly; (b) polar, negatively charged residues and their (uncharged) amides: Asp, Asn, Glu and Gln; (c) polar, positively charged residues: His, Arg and Lys; (d) large aliphatic, nonpolar residues: Met, Leu, Ile, Val and Cys; and (e) aromatic residues: Phe, Tyr and Trp.

More specifically, a conservative substitutions can be as follows: Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

A chitinolytic enzyme can be an endo-chitinase or an exo-chitinase. Preferably, the chitinolytic enzyme is capable of cleaving chitin that is present as a structural component of a fungus and/or an insect. A structural component of a fungus can be the cell wall. A structural component of an insect can be the exoskeleton. Most preferably, the chitinolytic enzyme is capable of cleaving chitin that is present in the cell wall of a fungus. The chitinolytic enzyme can further comprise a second amino acid sequence fused to the N-terminus of the first amino acid sequence. The second amino acid sequence is typically located at the N-terminus of the enzyme.

The second amino acid sequence preferably is less than 50 amino acids in length, more preferably less than 30, even more preferably less than 25 amino acids, such as 22 amino acids.

The second amino acid sequence is typically a sequence that causes secretion from a cell, such as a bacterial cell. Accordingly, the second amino acid can be a signal peptide. Specific examples of the second amino acid sequence include a natural signal peptide, a PelB signal peptide (SEQ ID NO: 6) or a dsbA protein. Preferably, the second amino acid sequence is the PelB signal peptide.

The chitinolytic enzyme can further comprise a third amino acid sequence fused to C-terminus to the first amino acid sequence. The third amino acid sequence is typically located at the C-terminus of the enzyme.

The third amino acid sequence is preferably less than 50 amino acids in length, more preferably less than 30, even more preferably less than 20 amino acids. Most preferably, the third amino acid sequence is less than 10 amino acids in length, such as 6 amino acids.

The third amino acid sequence is typically a sequence that facilitates purification of the enzyme after production by a cell, such as a bacterial cell. Accordingly, the third amino acid can be a purification tag. Specific examples of a purification tag include a 6×His tag (SEQ ID NOs: 7) or a Tag54/6×His combi-tag). Preferably, the third amino acid sequence is a 6×His tag.

The present inventors surprisingly found that the use of a (N-terminal) PelB signal peptide and a (C-terminal) 6×His tag achieved optimized production yields of the chitinolytic enzymes. The invention thus also provides a chitinolytic enzyme comprising or (essentially) consisting of a first amino acid sequence, a second amino acid sequence and a third amino acid sequence, wherein the second amino acid sequence is a PelB signal peptide and the third amino acid sequence is a 6×His tag.

The chitinolytic enzyme is preferably a purified chitinolytic enzyme. “Purified” in this context means that less than 5% of impurities are present, such as less than 2% or even less than 1% impurities. Impurities in this context means any substances other than the enzyme and optionally a solvent.

### Nucleic Acid, Vector and Host (Cell)

The invention also relates to a nucleic acid encoding a chitinolytic enzyme. More specifically, the invention provides a nucleic acid encoding a chitinolytic enzyme as described herein. For example, the nucleic acid can comprise a nucleotide sequence that is at least 50% identical (such as 100% identical) to a nucleotide sequence selected from the group of SEQ ID NOs: 8-12. For example, the nucleotide sequence is at least 60%, at least 70%, at least 80%, at least 90%, at least 95%, or at least 99% identical to a nucleotide sequence selected from the group of SEQ ID NOs: 8-12.

A nucleic acid encoding a chitinolytic enzyme can also encode more than one chitinolytic enzyme as described herein. Thus, the invention provides a nucleic acid encoding a chitinolytic enzyme comprising a first amino acid sequence that is at least 70% identical (such as 100% identical) to SEQ ID NO: 1 and a chitinolytic enzyme comprising a first amino acid sequence that is at least 70% identical (such as 100% identical) to SEQ ID NO: 2, and optionally a chitinolytic enzyme comprising a first amino acid sequence that is at least 70% identical (such as 100% identical) to SEQ ID NO: 3.

A nucleic acid may be for example DNA, RNA, or a hybrid thereof, and may also comprise (e.g. chemically) modified nucleotides, like PNA. It can be single- or double-stranded DNA. For example, the nucleotide sequences of the present disclosure may be genomic DNA, cDNA.

The invention further provides a vector comprising the nucleic acid encoding a chitinolytic enzyme. A vector as used herein is a vehicle suitable for carrying genetic material into a cell. A vector includes naked nucleic acids, such as plasmids or mRNAs, or nucleic acids embedded into a bigger structure, such as liposomes or viral vectors.

Vectors generally comprise at least one nucleic acid that is optionally linked to one or more regulatory elements, such as for example one or more suitable promoter(s), enhancer(s), terminator(s), etc.). The vector can is an expression vector, i.e. a vector suitable for expressing an encoded polypeptide or construct under suitable conditions, e.g. when the vector is introduced into a (e.g. bacterial or plant) cell. For DNA-based vectors, this usually includes the presence of elements for transcription (e.g. a promoter and a polyA signal) and translation (e.g. Kozak sequence).

In the vector, said at least one nucleic acid and said regulatory elements can be “operably linked” to each other, by which is generally meant that they are in a functional relationship with each other. For instance, a promoter is considered “operably linked” to a coding sequence if said promoter is able to initiate or otherwise control/regulate the transcription and/or the expression of a coding sequence (in which said coding sequence should be understood as being “under the control of” said promotor).

Also, preferably the nucleic acid encoding a chitinolytic enzyme may constitute part of an expression system, wherein the nucleic acid represents an open reading frame. The open reading frame may be codon-optimized for a particular organism.

The invention further provides a (non-human) host or a host cell that comprises the nucleic acid or the vector. A suitable host cell can be a plant cell or microbial cell.

For example, a plant cell from an agricultural or ornamental plant can be used. A microbial cell can be, for example, a yeast or bacterial cell, such as E.Coli. An example of a suitable yeast is Pichia pastoris.

Also provided is a plant comprising a chitinolytic enzyme as described herein, a nucleic acid encoding the same, or a vector comprising the nucleic acid. Preferably, the nucleic acid or vector may be comprised in the genome of the plant. Examples of plants include arable crops, fruit-bearing plants or vegetables. Examples of plants include cereals, maize, oil seed rape, rice, soy bean or potato.

### Production Method

The invention also provides a method for producing a chitinolytic enzyme as described herein. Typically, the method comprises at least the step of culturing a host cell as described herein, and in particular a bacterial host cell, such as E. coli. The culture can be conducted in a medium suitable for growth of the host cell.

The method can further comprise a step of harvesting the host cell and/or the culture supernatant during and/or after the culture. Preferably, the supernatant is harvested after (a suitable period of) the culture.

The method can further comprise a step of purifying the chitinolytic enzyme. For example, the chitinolytic enzyme can be purified from the culture supernatant by an initial ammonium sulfate precipitation step and a sequential immobilized metal affinity chromatography purification of solubilized protein precipitate.

A method for producing a chitinolytic enzyme can, for example, comprise setting up an expression system comprising culturing a host cell which expresses one or more chitinolytic enzymes.

Preferably the chitinolytic enzyme produced by the method comprises N- and/or C-terminal modifications as described herein to facilitate secretion of the enzyme into the culture medium and/or purification from a culture supernatant. For example, the chitinolytic enzyme produced by the method can comprise a second and/or a third amino acid sequence as described herein.

Accordingly, the method can comprise purifying the chitinolytic enzyme from the culture supernatant by an initial ammonium sulfate precipitation step, wherein a sequential immobilized metal affinity chromatography purification of solubilized protein precipitate is performed using an amino acid tag, such as a 6×His tag, comprised in the enzyme.

### Composition

The invention also provides a composition comprising at least one of the chitinolytic enzyme as described herein.

Preferably the composition comprises at least two different chitinolytic enzymes as described herein.

Advantageously, the at least two different chitinolytic enzymes can be chosen such that they exert a synergistic effect, such as a disproportionally improved chitin degradation rate (compared to each of the chitinolytic enzymes individually).

The composition may for example comprise a chitinolytic enzyme as described herein comprising SEQ ID NO: 1 and a chitinolytic enzyme as described herein comprising SEQ ID NO: 2.

The composition may for example comprise a chitinolytic enzyme as described herein essentially consisting of SEQ ID NO: 1 and a chitinolytic enzyme as described herein essentially consisting of SEQ ID NO: 2.

The composition may for example comprise a chitinolytic enzyme as described herein consisting of SEQ ID NO: 1 and a chitinolytic enzyme as described herein consisting of SEQ ID NO: 2.

The composition may also for example comprise a chitinolytic enzyme comprising SEQ ID NO: 1, a chitinolytic enzyme comprising SEQ ID NO: 2 and a chitinolytic enzyme comprising SEQ ID NO: 3.

The composition may also for example comprise a chitinolytic enzyme essentially consisting of SEQ ID NO: 1, a chitinolytic enzyme essentially consisting of SEQ ID NO: 2 and a chitinolytic enzyme essentially consisting of SEQ ID NO: 3.

The composition may also for example comprise a chitinolytic enzyme consisting of SEQ ID NO: 1, a chitinolytic enzyme consisting of SEQ ID NO: 2 and a chitinolytic enzyme consisting of SEQ ID NO: 3.

In each of these embodiments, the composition preferably comprises a chitinolytic enzyme comprising or (essentially) consisting of SEQ ID NO: 1 at higher amounts than the other chitinolytic enzymes.

Such synergistic combinations are particularly useful for use as plant protection agent as described herein.

The composition can be a liquid or a dry composition, preferably a liquid composition. A liquid composition can suitably be an aqueous composition.

The concentrations of the chitinolytic enzyme in the composition may be e.g. 0.01 mg/L to 250 g/L, such as 0.025 mg/L to 100 g/L. Specific examples of concentrations are as follows, according to applications as further described herein:
Insecticidal application: 0.01% to 5% (w/v), such as 0.05% to 2.5% (w/v), preferably 0.1 to 1% (w/v)
Fungicidal application: 0.25 μg/100 μl to 25.0 μg/100 μl, such as 0.7 μg/100 μl to 15.0 μg/100 μl, preferably 1.25 μg/100 μl to 10.0 μg/100 μl
Indirect fungicidal application: 0.25 μg/100 μl to 25.0 μg/100 μl, such as 0.7 μg/100 μl to 15.0 μg/100 μl, preferably 0.65 μg/100 μl to 5 μg/100 μl
Abiotic stress: 0.01 mg/L to 1 mg/L, such as 0.025 mg/L to 0.5 mg/L, preferably 0.05 mg/L to 0.25 mg/L

Preferably, the composition does not comprise an inhibitor of chitinolytic activity. Such inhibitors maybe metallic ions (for example, divalent ions, such as Zn2+, Cu2+, Ni2+), detergents (for example, sodium dodecyl sulfate (SDS), Triton X100 or Polysorbate 20), or certain other chemicals (for example, EDTA, imidazole). Thus, for example, the composition does not comprise metallic ions and/or SDS.

### Plant Protection

The present inventors have surprisingly found that chitinolytic enzymes can be used for protecting plants from pests such as fungi.

Moreover, optimum temperatures for the chitinolytic enzymes described herein (30-40° C.) were found lower than those reported in the literature for bacterial chitinases, as these enzymes are predominantly reported as thermophilic or thermo-tolerant enzymes with optima in the 40-60° C. range. This makes the chitinolytic enzymes newly described herein particularly suitable for applications on plants at ambient temperatures.

The inventors further surprisingly found that the chitinolytic enzymes newly described herein can degrade crystalline chitin, exemplified by chitin powder. Powdery chitin reflects realistic process parameters for later applications of the enzymes in degradation processes. Thus, the capacity of the chitinolytic enzymes described herein to degrade crystalline chitin makes them highly suitable for degrading chitin as present in pests.

The present invention thus provides a composition comprising at least one chitinolytic enzyme as described herein that is a plant protection agent. The present invention further provides a composition comprising at least two chitinolytic enzymes as described herein that is a plant protection agent.

The present invention also provides a use of a composition comprising at least one chitinolytic enzyme as described herein as a plant protection agent. The present invention also provides a use of a composition comprising at least two chitinolytic enzymes as described herein as a plant protection agent.

The plant protection agent is preferably against an organism that contains chitin. For example, chitin is a primary component of cell walls of fungi, the exoskeletons of arthropods, such as insects and the radulae of mollusks. Accordingly, the plant protection agent can be against infestation of fungi, insects or mollusks, and preferably fungi or insects, most preferably fungi.

Examples of fungi include ascornycetes, e.g. from the family Nectriaceae or Mycosphaerellaceae. Examples of fungi from the family of Nectriaceae include fungi from the genus Fusarium, such as Fusiarum oxysporum, Fusiarum graminearum, Fusarium culmorum. Examples of fungi from the family Mycosphaerellaceae include fungi from the genus Septoia, such as Septoria trifici. Other examples include Alternaria solani, Phytophtora infestans, Pythium, Magnaporthe oryzae, Venturia inaequalis, Pyrenophora teres, Rhynchospodum secalis, Puccinia triticina and Ramularia collo-cygni. The fungi can be filamentous fungi. The fungi are typically pathogenic fungi.

Examples of insects include insects from the family Aphididae, Tenebrionidae, Drosophilidae or Aphrophoridae, Examples of insects from the family of Aphididae include insects from the genus Sitabion, such as Sitabion avanae. Examples of insects from the family of Tenebrionidae include insects from the genus Tribolium, such as Tribolium castaneum. Examples of insects from the family of Drosophilidae include insects from the genus Drosophila, such as Drosophila melanogaster. Examples of insects from the family of Aphrophoridae include insects from the genus Philaenus, such as Philaenus spurnaius.

The plant to be protected is not particularly limited, and includes, for example, arable crops, fruit-bearing plants or vegetables.

Examples of plants include cereals, corn, oil seed rape, rice, barley, soy bean, pear, cherry, apple or potato. Preferably, the plant is corn, rice, barley, pear or cherry.

The invention further provides a method of protecting a plant from pests, the method comprising the application of a chitinolytic enzyme or a composition comprising at least one chitinolytic enzyme, such as a chitinolytic enzyme as described herein, on the plant or a part thereof. Typically, the composition is applied to the surface of the plant or a part thereof.

Pests include, for example, fungi, insects or mollusks. Examples thereof are given above. Preferably, the pest is a fungus or an insect, most preferably a fungus.

The application of the chitinolytic enzyme or the composition can for example comprise soaking of a plant or a plant part in a composition as described herein. Another exemplary application of the chitinolytic enzymes can comprise spraying a composition as described herein onto a plant or a plant part.

A plant part can be, for example, a leaf, a fruit or a seed (such as a grain). A seed can be a coated or an uncoated seed. Coated seed technology is commonly known and readily amendable to a person skilled in the art.

The pest can suitably be an organism that contains chitin. Accordingly, the pest can be, for example, a fungus, an insect or a mollusk, and preferably a fungus. Examples of such organisms are described above.

The plant to be protected is not particularly limited, and includes, for example, arable crops, fruit-bearing plants or vegetables. Examples are described above.

For any application as plant protection agent, a composition comprising at least two chitinolytic enzymes as described herein is preferably used.

Plant protection may also be achieved by expressing at least one chitinolytic enzyme as described herein in a plant or plant cell. Accordingly, the invention also provides a use of the nucleic acid or the vector as described herein for expressing a chitinolytic enzyme in a plant or plant cells. Expression may be constitutive or inducible. For example, the expression may be inducible in response to external stimuli, e.g. pest infestation (for example, via endogenous sensory mechanisms in the plant that can detect tissue damage).

The chitinolytic enzymes or composition comprising the same as described herein can be suitably used at a temperature of 0-40° C., such as 10-40 C, 20-40° C. or preferably 25-35° C.

The chitinolytic enzymes or composition comprising the same as described herein can be suitably used at a pH value of 4-11, such as 5-10, 6-10, 7-10 or 8-10.

The chitinolytic enzymes or composition comprising the same as described herein can be suitably used at a salinity of 0-20% or 0-10% or 0-5%.

Plant protection may also refer to protection from abiotic stress. Abiotic stress includes, for example, frost, drought, salt, flooding or heat stress. Preferably, the abiotic stress is frost stress or drought stress.

The invention thus further provides a method of protecting a plant from abiotic stress, the method comprising the application of a chitinolytic enzyme or a composition comprising at least one chitinolytic enzyme, such as a chitinolytic enzyme as described herein, on the plant or a part thereof. Typically, the composition is applied to the surface of the plant or a part thereof.

In the context of abiotic stress, a chitinolytic enzyme is preferably used, comprising or (essentially) consisting of a first amino acid sequence that is at least 70%, such as 100%, identical to an amino acid sequence of SEQ ID NO: 1, as further described herein. The description for the method of protecting a plant from pests applied mutatis mutandis.

### Further Agents

The chitinases can be suitably combined with further agents that can act as plant protection agents, e.g. agents against abiotic stress, fungicides and/or insecticides. Such further agents can be, for example, ascorbic acid, betaine or salicylic acid.

Thus, the invention also provides a composition comprising at least one chitinase and the uses thereof as described herein, further comprising ascorbic acid, betaine and/or salicylic acid.

Thus, the invention also provides a composition comprising at least one chitinase and the uses thereof as described herein, further comprising a fungicide and/or an insecticide.

### EXAMPLES

The following experimental section of the application relates to non-limiting exemplified embodiments of the present invention.

### Example 1

A novel marine chitinolytic bacterial strain, Photobacterium orarium, was isolated from coastal seawater samples and the initial genetic analysis of the 16S rDNA revealed that it belongs to the genus Photobacterium. The strain is capable to utilize marine chitin from shrimp shells as carbon and nitrogen source after carrying out an extracellular hydrolysis of chitin to GlcNAC and GlcNAC2 by secreting a cocktail of multiple chitinolytic enzymes.

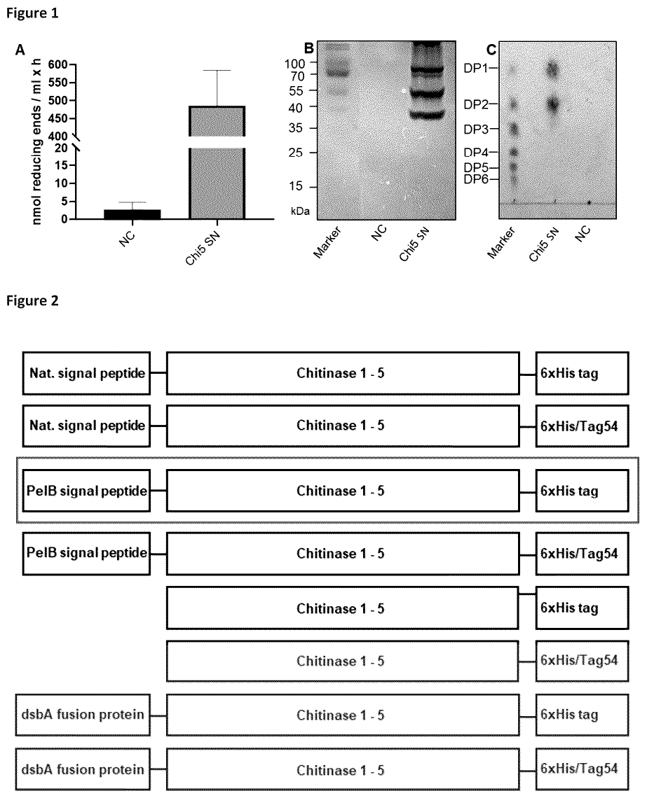

Chitinolytic activity of the culture supernatant of P. orarium was assessed using a reducing end assay, zymography as well as thin layer chromatography. All analytical methods confirmed that P. orarium is capable to degrade marine chitin. The reducing end assay demonstrated that, relative to the E. coli BL21 (DE3) negative control, substantial amounts of reducing sugars are released after incubation of the P. orarium supernatant with chitin powder for 16 h at 30° C. (FIG. 1A).

The release of free sugars is thus associated with the presence of chitinolytic enzymes that hydrolyze the glycosidic bonds within chitin. Zymography confirmed at least four chitinolytic enzymes that are involved in the degradation process ranging from ˜37 kDa to over 100 kDa (FIG. 1B). Product analysis by TLC further revealed that monomeric and dimeric compounds are generated (FIG. 1C) indicating the presence of exo-chitinase activity within the supernatant.

P. orarium proved to be an entirely novel bacterial strain and a source for enzymes with exo-chitinase activity that can be used for conversion of insoluble chitin to monomeric and dimeric sugars. In this respect, the characteristic of P. orarium to utilize chitin as a sole carbon and nitrogen source could point to novel and unique chitinolytic enzymes with high conversion rates for crystalline chitin.

### Example 2

### Introduction

Individual chitinases produced by P. orarium could be expressed recombinantiy and characterized separately. This example summarizes the gene mining of putative chitinases, their cloning and recombinant expression in E. coli BL21 and sequential characterization of the respective enzymes. Furthermore, soluble hydrolysis products are identified by thin layer chromatography by testing various chitin substrates and chitin standard molecules.

In order to employ chitinases for the production of chitosan oligomers (COS), a limited degradation of chitin by endo-chitinases must be ensured while avoiding a hydrolysis by exo-chitinases, chitobiases and N-acetyl-β-glucosarrinidases to GlcNAc. As P. orarium secretes a potent mixture of chitinolytic enzymes in order to depolymerize chitin to assimilable GlcNAC and GlcNAC2, the active culture supernatant cannot be utilized readily for the production of COS thus indicating the existence of exo-chitinase activity. In this example, all genes coding for chitinases were therefore identified from the P. orarium genome data and expressed recombinantly in order to yield single enzymes for the identification of endo- and exo-chitinases and for the determination of activity optima.

### Results

### A. Cloning and Expression of Putative Chitinases

Gene mining was carried out after next generation sequencing and 60 contigs were screened for chitinase homologues using the Pfam database (version 2.9). Five genes (C1-C5) were identified that showed several characteristic chitinase domains (Table 4.1). The chitinase A and glycosyl hydrolase 18, 19 and 20 domains have previously been reported to be specific for chitinases and chitobiases that differ from each other in structures and modes of action. Compared to the currently reported chitinases that range between 20-90 kDa, the predicted molecular weight of the novel chitinases in general is ranked high. C1, 3 and 4 are significantly larger with 83.9-86.8 kDa compared to C2 and 5 with 56.5 and 65.4 kDa, respectively. However, no additional or unknown domains were identified within the genes. Natural signal peptides (also termed natural leader sequences, NLs) were discovered for C1, C2, C3 and C4, suggesting that C5, containing also a putative chitobiase domain, remains intracellular in order to further hydrolyze GlcNAG2 to GlcNAG.

TABLE 4.1
Chitinase specific domains for the five putative chitinases (C1-C5) that
were identified in the genome of Chi5. AA = amino acid. kDa = kilo dalton.

| | Size | Molecular | | | | Signal |
|---|---|---|---|---|---|---|
| Enzyme | AA | weight [kDa] | Domain 1 | Domain 2 | Domain 3 | peptide |
| C1 | 815 | 85.5 | Chitinase A, N- | Glycosyl hydrolase | — | ✓ |
| | | | terminal domain | family 18 | | |
| C2 | 515 | 56.5 | Carbohydrate | Glycosyl hydrolase | — | ✓ |
| | | | binding domain | family 19 | | |
| C3 | 826 | 86.8 | Chitinase A, N- | Glycosyl hydrolase | — | ✓ |
| | | | terminal domain | family 18 | | |
| C4 | 777 | 83.9 | Glycosyl hydrolase | Chitinase C | Cellulose- | ✓ |
| | | | family 18 | | binding domain | |
| C5 | 594 | 65.4 | Glycosyl hydrolase | Glycosyl hydrolase | Chitobiase | — |
| | | | family 20, domain 2 | family 20, catalytic | domain | |
| | | | | domain | | |

A total of eight constructs comprising a cytosolic form, a fusion to the dsbA protein, variants with the natural and PelB signal peptide and the 6×His tag as well as Tag54/6×His combi-tag were cloned for each putative chitinase gene (FIG. 2) in E. coli DH5a.

TABLE X
The amino acid sequences used in these constructs

| | SEQ ID | |
|---|---|---|
| Name | NO | Sequence |
| Chitinase 1 | 1 | MAAPGTPQIAWMETDYAIVEVDQAASAYKSLITVKPAAEVPVAWQRYSGETADLWKV |
| | | KLNGNVVFEQSIAPASSGAGSTTLSVAQGGQYAMTVELCSGTGAAQACTSSAATNIV |
| | | VADTDGSHLDPLPMNVDANNGNYTTPANTWVGAYFVEWGVYGRKFSVDNIPAQNLT |
| | | HILYGFIPICGPNESLGEIENGNSLAALNRACQGTPDYEVVIHDPWAAVQMPQPQSGH |
| | | THSTPYKGTYGQMMALKQRYPDLKIVPSVGGWTLSDPFYSFTDKAKRDIFVASVKKFL |
| | | KTWKFFDGVDIDWEFPGGKGANANLGDPVNDGPAYVALMRELRVMLDELEAETGRQ |
| | | YELTSAIGVGYDKIDVVNYADAVQYMDYIFAMTYDFFGGWNNVPGHQTALFCGSHMS |
| | | ADVCNGTGVDENGVPRQGPAYTAAHGIDRLLAQGVPANKLVLGTAMYARGWTGVTE |
| | | ASMTDPTNPMTGTGNGMVPGSWEPGVIDYKDVVTDYINNAAVTKGYDAQAEAPWAY |
| | | NPSNGNLITYDDRRSVLAKGQYVRNLGLAGLFAWEIDADNGDILNAMQDSLAGGGGN |
| | | YAPVAKAGADQVVNGAASVTLDGSASSDRDGQIVSYQWTQTSGPALTLTGSDTATAT |
| | | VAVPEVTADTQYAFTLTVTDNAGDTATDSVTVTAKAPGSNTAPVVAVSGPATASAGD |
| | | TVVLDASQSSDADGDTLTFTWTVPAGVNATVNGATVSFVADSYTVDTPLNFTVAVSD |
| | | GTDSVTESVTVTVLKDGGSTGCTNAWATGGVYNSGDIVTHSGKSWKAKWWTTGEE |
| | | PGTTGEWGVWEDLGAANCQIE |
| Chitinase 2 | 2 | MATANYNPDGVYSAGEQVIYQGATYQALRSTENEAPDTYLGDAWQQVNAQNVTSST |
| | | TPTYPAYQSSAVYVGGDHVSYNSQIYKAKWWTQGEAPDATPGTGVWEWVSADNNP |
| | | DPGPGPDPDPTPEPTPANGIIGQNPDGSYIMSKTYLDAREAELTSSPEFAAVLASISTR |
| | | DNAWVEAWPGLSTNPDNVKRVEALISEQKWDALFPERNVVYTYSNFLKAVAKFKGFC |
| | | ATYTDERAAQSDAICAKSLAVMFAHFTQETGAHNPHSPYEEWRQGLFFVREAGCSEE |
| | | ASSCGYNSECAASNWQTEQWPCGTNPDGSYVKYFGRGAKQLSYHYNYGPFSDFIFN |
| | | DVNVLLQDPDRVADSWLNLASAVFFFVYPQPPKPSMLHVIDGTWQPTAADIAEKRVP |
| | | GFGVTTMIINGGIECTLDTEKPQSVNRIKYYRGHAAALGVAIPADEQLGCAGMKAFKKT |
| | | GDSTFGLYWENDWSYYPDNPGGSSFACRIESGYQTAHTTLKKGDYAKCIQKYYGVTI |
| | | E |
| Chitinase 3 | 3 | MALAAPGTPQLAWMETDYAIVEVDQAATAYKDLVTVKNAADVPVAWQRYSGETADH |
| | | WKVKLNGNVAFEESINPASSGAGSTTLSITQGGQYALTVELCSGSGAEEACSSSPATN |
| | | IVVADTDGSHLDPLPMNVDPNNGTYSTPADTVVGAYFVEWGVFGRKFTVDKIPAQNL |
| | | THILYGFIPVCGPNESLGEIESGNSLAALNRACAGTPDFEVAIHDPWAAVQMPQPQSG |
| | | HTYSTPYKGNYGQLMALKQRYPDLKVIPSIGGWTLSDPFFSFTDKSKRDVFVASVKKF |
| | | LQTWKFFDGVDIDWEFPGGKGANTTLGDPVNDGPAYVALMRELRTMLDGLEAETGR |
| | | QFELTSAIGVGYDKIDWNYAEASQYMDYIFNMSYDFYGGWSNVTGHQTALNCGSHL |
| | | TADQCNGTGVDENGEPRQGPAYTTAHGVERLLAQGVPANKLVVGAAMYGRGWTGV |
| | | TQASMTDPSNPMTGVGNGAVAGSWEAGVIDYKDVTRYENKAGVVLGYDEQAEAP |
| | | WAYDPSNGDLVTYDSPRSIMAKGQYVRDLGLAGLFAWEIDADNGDILNAMQESLAGA |
| | | PAGNRAPVARAGSDQQVNTAATVTLNGSSSTDSDGQIAGYQWVQTSGPALTLNGAN |
| | | TASATISVPDVTVDTQYVFTLTVTDNEGATATDSITVTAKAPGAQNTAPVASLTGPGTA |
| | | NADDVITLDASQSTDADGDTLTYDWTVPAGVNAVINGSSLSFTADSYTTDTALSFSVS |
| | | VSDGTASDAASLTVTIAKDESGTGGEGDFPAYVEGTAYQAGDQVSNGGVNFECKPY |
| | | PYSGWCSGAAWAYEPGVGVYWQDAWTQLIE |
| Chitinase 4 | 4 | MAQAAANCRPDGLYQTPGVTVPYCTVYDQDGREKMGVDHPRRVIGYFTSWRSGND |
| | | PQSSYLVNDIPWDSLTHINYAFVSIGSDGKVNIGNVNNPDNPAVGKEWPGVEIDPALG |
| | | FKGHFGALATAKAQHGVKTLISIGGWAETGGHFDDNGNRVADGGFYTMTTNADGSIN |
| | | HQGIQTFADSAVAMMRQYKFDGLDIDYEYPTSMQGAGNPDDFAYSDAMRPHLMKSY |
| | | HELMKVLREKLDQASAQDGHHYMLTIAAPSSGYLLRGMETMSVTKYLDYVNIMSYDL |
| | | HGAWNDYVGHNASLFDSGLDAELEAGNVYGTAQYKKTGYLNTDWAYHYFRGSMPA |
| | | GRINIGVPYYTRGWQGVTGGTNGLWGKAALPNQSDCPTGTGAGTSNCGYGAIGIDN |
| | | MWHDKDANGNEMGAGSNPMWHAMNLANGVYGSYTAAYGLDPVNDPQDALTGTYT |
| | | RHYDSVSVAPWLWNADKKVFLSTEDKESINTKADYVIEQGIGGIMFWELAGDYSCYNL |
| | | DANGNRTTVDPTENACKTGNGEYHMGDTMTKAIHNKFATATPYGNTLAEGAIPTEAV |
| | | NIDVSVTGFKVGDQNYPLNPTMTLTNKTGQTLPGGTEFQFDIPTSTPDNMSDQSGAN |
| | | LQVISSGHTRGDNIGGLDGNFHRVAFTLPSWKQLGNNESFELTLNYFLPISGPANYSV |
| | | RVNNADYALAFEQPDLPIADLSNGGGDNGGGDGNNGDCVTTGVNTYPNWPQTDWA |
| | | GNPSHAGTGDKIIYNGAVYQAKWWTNSVPGSDGSWDTVCTVQIE |
| Chitinase 5 | 5 | MAMGWSQININGVQKWEEAYSLNVDAANEIITIGAADTAGALYASQSLLQLVDGNKVP |
| | | EVQITDAPRFAYRGFSVDAVRNFRTKDAIIQLLDQMAAFKLNKLHLRLADDEGWRIEIA |
| | | GLPELTDVGATRCHDPEEKACILPFLGAGPDGSPESNGYYTATDYQDILSHAAALNIEV |
| | | VPEIDIPGHAHAAIKAMEARYDHYAAQGNMAEANKYLLTDFNDTTQYLSVQMFTDNAI |
| | | NVCMESSYNFVDAVVSGLVSLHQGVQPLKTFHFGGDEIAGAWINSPACQDFIANNTD |
| | | GVHSVSDLSRYFVERISVITANYGLDMAGWEDGLMHDGQVYPRSQMANNQLWGNA |
| | | WQNIWEWGVADRAYNLANNDYKWVYNHATHLYFDHPYEPDPNERGYYWAPRFTDT |
| | | RKTFGFMPDDVFANADFTRAGAPITKAEVVASAGVKKLLKPENVLGLQGSLWAEAVR |
| | | TEDQFEGMIFPRVLGLAERAWHTATWEANDNAGIALDETGRNADYNHFANLLGQKVL |
| | | PKLEQAGIAFNLPVPGGVIENGVLQANSTFPGLTIEYSTDQGTSWQSYDHLNPPAVAA |
| | | GVQLRTVSGQRTSRVTTVNIE |
| PelB | 6 | MKYLLPTAAAGLLLLAAQPA |
| 6xHis tag | 7 | HHHHHH |

TABLE Y
The nucleotide sequences used encoding the chitinases

| | SEQ ID | |
|---|---|---|
| Name | NO | Sequence |
| Chitinase | 8 | ATGGCCGCACCTGGTACACCGCAGATTGCATGGATGGAAACCGATTATGCAATTGT |
| 1 | | TGAAGTTGATCAGGCAGCAAGCGCATATAAAAGCCTGATTACCGTTAAACCGGCAG |
| | | CCGAAGTTCCGGTTGCATGGCAGCGTTATAGCGGTGAAACCGCAGATCTGTGGAA |
| | | AGTTAAACTGAATGGTAACGTGGTTTTCGAGCAGAGCATTGCACCGGCAAGCAGC |
| | | GGTGCAGGTAGCACCACACTGAGCGTTGCACAAGGTGGTCAGTATGCAATGACCG |
| | | TTGAACTGTGTAGCGGTACAGGTGCAGCACAGGCATGTACCAGCAGCGCAGCAAC |
| | | CAATATTGTTGTTGCAGATACCGATGGTAGCCATCTGGATCCGCTGCCGATGAATG |
| | | TTGATGCAAATAATGGTAATTATACCACGCCTGCCAATACCGTTGTTGGTGCATATT |
| | | TTGTTGAATGGGGTGTGTATGGTCGTAAATTTAGCGTTGATAATATCCCTGCACAG |
| | | AATCTGACCCATATTCTGTATGGTTTTATTCCGATTTGCGGTCCGAATGAAAGCCTG |
| | | GGTGAAATTGAAAATGGTAATAGCCTGGCAGCACTGAATCGTGCATGTCAGGGTAC |
| | | ACCGGATTATGAAGTTGTTATTCATGATCCGTGGGCAGCAGTTCAGATGCCGCAGC |
| | | CGCAGAGCGGTCATACCCATAGCACCCCGTATAAAGGCACCTATGGTCAGATGAT |
| | | GGCACTGAAACAGCGTTATCCGGATCTGAAAATTGTTCCGAGCGTTGGTGGTTGG |
| | | ACCCTGAGCGATCCGTTTTATAGCTTTACCGATAAAGCCAAACGCGATATCTTTGTT |
| | | GCAAGCGTGAAAAAGTTTCTGAAAACCTGGAAATTTTTCGACGGCGTGGATATCGA |
| | | TTGGGAATTTCCTGGTGGTAAAGGTGCAAATGCAAATCTGGGTGATCCGGTTAATG |
| | | ATGGTCCGGCATATGTTGCACTGATGCGTGAACTGCGTGTTATGCTGGATGAACTG |
| | | GAAGCAGAAACCGGTCGTCAGTATGAACTGACAAGCGCAATTGGTGTTGGCTATG |
| | | ACAAAATTGATGTGGTGAATTATGCAGACGCCGTGCAGTATATGGATTATATCTTTG |
| | | CCATGACCTATGATTTTTTCGGTGGCTGGAATAATGTTCCGGGTCATCAGACCGCA |
| | | CTGTTTTGCGGTAGCCATATGAGCGCAGATGTTTGTAATGGCACCGGTGTTGATGA |
| | | AAATGGCGTTCCGCGTCAGGGTCCTGCATATACCGCAGCACATGGTATTGATCGT |
| | | CTGCTGGCACAGGGTGTTCCGGCAAATAAACTGGTTCTGGGCACCGCAATGTATG |
| | | CACGCGGTTGGACCGGTGTTACCGAAGCAAGCATGACCGATCCGACCAATCCGAT |
| | | GACCGGCACCGGTAATGGTATGGTTCCTGGTAGCTGGGAACCGGGTGTTATTGAT |
| | | TATAAAGATGTCGTGACCGACTATATCAATAATGCAGCAGTTACCAAAGGTTATGAT |
| | | GCACAGGCCGAAGCACCGTGGGCATATAATCCGAGCAATGGCAATCTGATTACCT |
| | | ATGATGATCGTCGTAGCGTTCTGGCAAAAGGCCAGTATGTGCGTAATCTGGGTTTA |
| | | GCAGGTCTGTTTGCATGGGAAATTGATGCCGATAATGGCGATATTCTGAATGCAAT |
| | | GCAGGATAGTCTGGCAGGCGGTGGCGGTAATTATGCACCGGTGGCAAAAGCCGG |
| | | TGCAGATCAGGTTGTTAATGGTGCAGCCAGCGTTACCCTGGATGGTAGCGCGAGC |
| | | AGCGATCGTGATGGCCAGATTGTTAGCTATCAGTGGACCCAGACCAGCGGTCCGG |
| | | CACTGACCCTGACCGGTAGCGATACCGCAACCGCGACCGTTGCAGTTCCGGAAGT |
| | | TACCGCAGATACCCAGTATGCATTTACACTGACCGTTACAGATAATGCCGGTGATA |
| | | CAGCAACCGATAGCGTTACCGTTACCGCCAAAGCACCGGGTAGTAATACCGCACC |
| | | GGTTGTTGCCGTTTCAGGTCCGGCAACCGCAAGCGCAGGCGATACCGTGGTTCTG |
| | | GATGCAAGCCAGAGCAGTGATGCAGATGGCGATACCCTGACCTTTACCTGGACCG |
| | | TTCCGGCAGGCGTTAATGCAACCGTGAATGGTGCAACCGTTAGCTTTGTTGCCGAT |
| | | AGCTATACCGTGGATACACCGCTGAATTTTACCGTTGCCGTTAGTGATGGTACAGA |
| | | TAGCGTGACCGAAAGTGTGACCGTGACCGTTCTGAAAGATGGTGGTAGTACCGGT |
| | | TGTACCAATGCATGGGCAACCGGTGGTGTGTATAATAGTGGTGATATTGTGACCCA |
| | | TAGCGGCAAAAGCTGGAAAGCAAAATGGTGGACCACCGGTGAAGAACCGGGTAC |
| | | GACAGGTGAATGGGGAGTTTGGGAAGATCTGGGTGCAGCAAATTGTCAGATCGAA |
| Chitinase | 9 | ATGGCCACCGCAAACTATAATCCGGATGGTGTTTATAGTGCCGGTGAACAGGTTAT |
| 2 | | TTATCAGGGTGCAACCTATCAGGCACTGCGTAGCACCGAAAATGAAGCACCGGAT |
| | | ACCTATCTGGGTGATGCATGGCAGCAGGTTAATGCACAGAATGTTACCAGCAGCA |
| | | CCACCCCGACCTATCCGGCATATCAGAGCAGCGCAGTTTATGTTGGTGGTGATCAT |
| | | GTTAGCTATAACAGCCAGATCTATAAAGCAAAATGGTGGACCCAGGGTGAAGCTCC |
| | | GGATGCAACACCGGGTACAGGTGTTTGGGAATGGGTTAGCGCAGATAATAATCCT |
| | | GATCCGGGTCCTGGTCCGGATCCAGATCCGACACCGGAACCGACTCCGGCAAAT |
| | | GGTATTATTGGTCAGAACCCGGATGGTAGCTATATTATGAGCAAAACATATCTGGA |
| | | TGCACGTGAAGCAGAACTGACCAGCAGTCCGGAATTTGCAGCAGTTCTGGCAAGC |
| | | ATTAGCACCCGTGATAATGCAGTTGTTGAAGCAGTTGTTCCGGGTCTGAGCACCAA |
| | | TCCGGATAATGTTAAACGTGTTGAAGCCCTGATTAGCGAACAGAAATGGGATGCAC |
| | | TGTTTCCGGAACGTAATGTTGTTTATACCTATAGCAACTTCCTGAAAGCCGTGGCAA |
| | | AATTCAAAGGTTTTTGTGCCACCTATACCGATGAACGTGCAGCACAGAGTGATGCA |
| | | ATTTGTGCAAAAAGCCTGGCAGTTATGTTTGCCCATTTTACCCAAGAAACCGGTGC |
| | | ACATAATCCGCATAGCCCGTATGAAGAATGGCGTCAGGGTCTGTTTTTTGTTCGTG |
| | | AAGCCGGTTGTAGCGAAGAAGCAAGCAGCTGCGGTTATAATAGCGAATGTGCAGC |
| | | AAGCAATTGGCAGACCGAACAGTGGCCGTGTGGTACAAATCCTGATGGTAGTTAT |
| | | GTGAAATACTTTGGTCGTGGTGCAAAACAGCTGAGCTATCATTATAACTATGGTCC |
| | | GTTTAGCGACTTCATCTTCAATGATGTTAATGTGCTGCTGCAGGACCCTGATCGTG |
| | | TTGCAGATAGCTGGCTGAATCTGGCAAGTGCAGTTTTTTTCTTTGTTTATCCGCAGC |
| | | CTCCGAAACCGAGCATGCTGCATGTTATTGATGGCACCTGGCAGCCGACCGCAGC |
| | | AGATATTGCAGAAAAACGCGTTCCGGGTTTTGGTGTTACCACCATGATTATTAACG |
| | | GTGGTATTGAATGTACCCTGGATACCGAAAAACCGCAGAGCGTTAATCGCATTAAA |
| | | TACTATCGTGGTCATGCAGCCGCACTGGGTGTTGCAATTCCGGCAGATGAACAGC |
| | | TGGGTTGTGCAGGTATGAAAGCATTCAAAAAAACCGGTGATAGCACCTTTGGTCTG |
| | | TATTGGGAAAATGATTGGAGCTATTATCCTGATAATCCGGGTGGCAGCAGCTTTGC |
| | | ATGTCGTATTGAAAGCGGTTATCAGACCGCACATACCACCCTGAAAAAAGGTGATT |
| | | ATGCCAAATGCATTCAGAAATATTACGGCGTGACCATCGAA |
| Chitinase | 10 | ATGGCCTTAGCAGCACCGGGTACACCGCAGCTGGCATGGATGGAAACCGATTATG |
| 3 | | CAATTGTTGAAGTTGATCAGGCAGCAACCGCATATAAAGATCTGGTTACCGTTAAA |
| | | AATGCAGCCGATGTTCCGGTTGCATGGCAGCGTTATAGCGGTGAAACCGCAGATC |
| | | ATTGGAAAGTTAAACTGAATGGTAACGTGGCCTTTGAAGAAAGCATTAATCCGGCA |
| | | AGCAGCGGTGCAGGTAGCACCACACTGAGCATTACCCAAGGTGGTCAGTATGCAC |
| | | TGACCGTTGAACTGTGTAGCGGTAGCGGTGCCGAAGAAGCATGTAGCAGCAGTCC |
| | | GGCAACCAATATTGTTGTTGCAGATACCGATGGTAGCCATCTGGATCCGCTGCCGA |
| | | TGAATGTTGATCCGAATAATGGCACCTATAGCACACCGGCAGATACAGTTGTTGGT |
| | | GCATATTTTGTTGAATGGGGTGTGTTTGGTCGCAAATTTACCGTTGATAAAATTCCG |
| | | GCACAGAATCTGACCCATATTCTGTATGGTTTTATTCCGGTTTGTGGTCCGAATGAA |
| | | AGCCTGGGTGAAATTGAAAGCGGTAATAGCCTGGCAGCACTGAATCGTGCATGTG |
| | | CCGGTACACCGGATTTTGAAGTTGCAATTCATGATCCGTGGGCAGCAGTTCAGATG |
| | | CCGCAGCCGCAGAGCGGTCATACCTATAGTACCCCGTATAAAGGTAATTATGGTCA |
| | | GCTGATGGCACTGAAACAGCGTTATCCGGATCTGAAAGTTATTCCGAGCATTGGTG |
| | | GTTGGACCCTGAGCGATCCGTTTTTTAGCTTTACCGATAAAAGCAAACGTGATGTTT |
| | | TTGTTGCCAGCGTGAAAAAATTCCTGCAGACCTGGAAATTTTTCGATGGCGTTGAT |
| | | ATCGATTGGGAATTTCCTGGTGGTAAAGGTGCAAATACCACACTGGGTGATCCGGT |
| | | TAATGATGGTCCGGCATATGTTGCACTGATGCGTGAACTGCGTACCATGCTGGATG |
| | | GTCTGGAAGCAGAAACCGGTCGTCAGTTTGAACTGACCAGCGCAATTGGTGTTGG |
| | | TTATGACAAAATTGATGTGGTGAATTATGCCGAAGCAAGCCAGTATATGGACTACAT |
| | | CTTTAACATGAGCTACGACTTTTATGGCGGTTGGAGCAATGTTACCGGTCATCAGA |
| | | CCGCACTGAATTGTGGTTCACATCTGACAGCAGATCAGTGTAATGGTACAGGTGTT |
| | | GATGAAAATGGTGAACCGCGTCAGGGTCCTGCATATACCACCGCACATGGTGTTG |
| | | AACGTCTGCTGGCACAGGGTGTTCCGGCAAATAAACTGGTTGTGGGTGCAGCAAT |
| | | GTATGGTCGCGGTTGGACCGGTGTTACCCAGGCAAGCATGACCGATCCGAGCAAT |
| | | CCGATGACAGGCGTTGGTAATGGTGCAGTTGCCGGTAGCTGGGAAGCCGGTGTTA |
| | | TTGATTACAAAGATGTTGTGACCCGCTATGAAAACAAAGCGGGTGTTGTTCTGGGT |
| | | TATGATGAACAGGCCGAAGCACCGTGGGCATATGATCCGTCAAATGGTGATCTGG |
| | | TGACCTATGATAGTCCGCGTAGCATTATGGCAAAAGGCCAGTATGTTCGTGATCTG |
| | | GGTTTAGCAGGTCTGTTTGCATGGGAAATTGATGCAGATAATGGCGATATTCTGAA |
| | | CGCAATGCAAGAAAGTCTGGCAGGCGCACCGGCAGGTAATCGTGCACCGGTTGC |
| | | GCGTGCGGGTAGCGATCAGCAGGTTAATACCGCAGCAACAGTTACCCTGAATGGT |
| | | TCAAGCAGTACCGATAGTGATGGTCAGATTGCAGGTTATCAGTGGGTTCAGACCAG |
| | | CGGTCCGGCACTGACGCTGAATGGTGCCAATACCGCAAGCGCCACCATTAGCGTT |
| | | CCGGATGTTACCGTGGATACCCAGTATGTGTTTACCCTGACCGTGACCGATAATGA |
| | | AGGTGCAACCGCAACCGATAGCATTACCGTTACCGCAAAAGCTCCGGGTGCACAG |
| | | AATACCGCACCGGTGGCCAGTCTGACCGGTCCGGGTACAGCAAATGCAGATGATG |
| | | TTATTACCCTGGATGCAAGCCAGAGCACCGATGCAGATGGTGATACCCTGACATAT |
| | | GATTGGACCGTTCCGGCAGGCGTTAATGCAGTTATTAATGGTAGCAGCCTGAGTTT |
| | | TACCGCAGATAGCTATACAACCGATACCGCACTGTCATTTAGCGTTAGCGTTTCAG |
| | | ATGGCACCGCAAGTGATGCAGCAAGCCTGACCGTTACAATTGCAAAAGATGAAAG |
| | | CGGCACAGGTGGTGAAGGTGATTTTCCTGCCTATGTTGAAGGCACCGCATATCAG |
| | | GCAGGCGATCAGGTTAGCAATGGTGGTGTTAATTTTGAGTGTAAACCGTATCCGTA |
| | | TAGCGGTTGGTGTAGTGGTGCAGCATGGGCCTATGAACCAGGTGTTGGTGTTTATT |
| | | GGCAGGATGCATGGACCCAGCTGATCGAA |
| Chitinase | 11 | ATGGCCCAGGCAGCAGCAAATTGTCGTCCGGATGGTCTGTATCAGACACCGGGTG |
| 4 | | TTACCGTTCCGTATTGTACCGTTTATGACCAGGATGGTCGTGAAAAAATGGGTGTT |
| | | GATCATCCGCGTCGTGTGATTGGTTATTTCACCAGCTGGCGTAGCGGTAATGATCC |
| | | GCAGAGCAGCTATCTGGTTAATGATATTCCGTGGGATAGTCTGACCCATATCAATT |
| | | ATGCATTTGTGAGCATTGGCAGTGATGGCAAAGTGAATATTGGCAATGTTAACAAT |
| | | CCGGATAATCCGGCAGTTGGTAAAGAATGGCCTGGTGTTGAAATTGATCCGGCAC |
| | | TGGGTTTTAAAGGTCATTTTGGTGCACTGGCAACCGCAAAAGCACAGCATGGTGTT |
| | | AAAACCCTGATTAGCATTGGTGGTTGGGCAGAAACCGGTGGCCATTTTGATGATAA |
| | | TGGTAATCGTGTTGCAGATGGTGGCTTTTATACCATGACAACCAATGCCGATGGTA |
| | | GCATTAATCATCAGGGTATTCAGACCTTTGCAGATAGCGCAGTTGCAATGATGCGT |
| | | CAGTATAAATTCGATGGCCTGGATATCGATTATGAATATCCGACCAGCATGCAAGG |
| | | TGCAGGTAATCCGGATGATTTTGCATATAGTGATGCAATGCGTCCGCATCTGATGA |
| | | AAAGCTATCATGAACTGATGAAAGTGCTGCGCGAAAAACTGGATCAGGCAAGCGC |
| | | ACAGGATGGACATCATTATATGCTGACCATTGCAGCACCGAGCAGCGGTTATCTGC |
| | | TGCGTGGTATGGAAACCATGAGCGTTACCAAATATCTGGACTATGTGAACATCATG |
| | | AGCTATGATCTGCATGGTGCATGGAATGATTATGTTGGTCATAATGCAAGCCTGTTT |
| | | GATAGCGGTCTGGATGCAGAACTGGAAGCAGGTAATGTTTATGGCACCGCACAGT |
| | | ACAAAAAAACCGGCTATCTGAATACAGATTGGGCCTATCATTATTTTCGTGGTAGCA |
| | | TGCCTGCAGGTCGTATTAACATTGGTGTGCCGTATTACACCCGTGGTTGGCAGGG |
| | | TGTGACCGGTGGCACCAATGGTCTGTGGGGTAAAGCAGCACTGCCGAATCAGAGC |
| | | GATTGTCCGACCGGTACAGGTGCAGGCACCAGCAATTGCGGTTATGGTGCAATTG |
| | | GTATTGATAACATGTGGCACGATAAAGATGCCAATGGTAATGAAATGGGTGCCGGT |
| | | AGCAATCCGATGTGGCATGCAATGAATCTGGCAAATGGTGTTTATGGTAGCTATAC |
| | | CGCAGCATATGGTCTGGATCCGGTTAACGATCCGCAGGATGCACTGACCGGTACG |
| | | TATACCCGTCATTATGACAGCGTTAGCGTTGCACCGTGGCTGTGGAATGCAGATAA |
| | | AAAAGTTTTTCTGAGCACCGAGGATAAAGAGAGCATTAATACCAAAGCCGATTACG |
| | | TGATTGAACAAGGTATTGGTGGTATCATGTTTTGGGAATTAGCCGGTGATTATAGCT |
| | | GCTACAATCTGGATGCGAATGGCAATCGTACCACCGTTGATCCGACCGAAAATGCA |
| | | TGTAAAACCGGTAATGGTGAATATCATATGGGTGACACCATGACCAAAGCCATCCA |
| | | TAACAAATTTGCAACAGCAACCCCGTATGGTAATACCCTGGCAGAAGGTGCAATTC |
| | | CGACGGAAGCAGTTAATATTGATGTTAGCGTTACCGGCTTTAAAGTGGGCGATCAG |
| | | AATTATCCGCTGAATCCGACCATGACACTGACCAATAAAACGGGTCAGACACTGCC |
| | | TGGTGGCACCGAATTTCAGTTTGATATTCCTACCAGCACACCGGATAATATGAGCG |
| | | ATCAGAGCGGTGCAAATCTGCAGGTTATTAGCAGCGGTCATACCCGTGGTGACAA |
| | | TATTGGTGGACTGGATGGCAATTTTCATCGTGTGGCATTTACCCTGCCGAGCTGGA |
| | | AACAGCTGGGTAATAATGAATCATTTGAACTGACCCTGAACTACTTTCTGCCGATTA |
| | | GCGGTCCGGCAAATTATAGCGTTCGTGTTAATAATGCAGATTATGCCCTGGCATTT |
| | | GAACAGCCGGATCTGCCTATTGCAGATCTGAGCAATGGTGGTGGTGATAACGGTG |
| | | GCGGTGATGGTAACAATGGTGATTGTGTTACCACCGGTGTTAATACCTATCCGAAT |
| | | TGGCCTCAGACCGATTGGGCAGGCAATCCGAGCCATGCAGGTACGGGTGATAAAA |
| | | TCATTTATAACGGTGCAGTGTATCAGGCAAAATGGTGGACCAATAGCGTTCCGGGT |
| | | AGTGATGGTAGCTGGGATACCGTTTGCACCGTTCAGATCGAA |
| Chitinase | 12 | ATGGCCATGGGTTGGAGCCAGATTAACATTAATGGTGTTCAGAAATGGGAAGAGG |
| 5 | | CCTATAGCCTGAATGTTGATGCAGCAAATGAAATCATTACCATTGGTGCAGCAGAT |
| | | ACCGCAGGCGCACTGTATGCAAGCCAGAGCCTGCTGCAGCTGGTTGATGGTAATA |
| | | AAGTTCCGGAAGTTCAGATTACCGATGCACCGCGTTTTGCATATCGTGGTTTTAGC |
| | | GTTGATGCCGTTCGTAATTTTCGTACCAAAGATGCAATTATTCAGCTGCTGGATCAG |
| | | ATGGCAGCATTCAAACTGAATAAACTGCATCTGCGTCTGGCAGATGATGAAGGTTG |
| | | GCGTATTGAAATTGCAGGTCTGCCGGAACTGACCGATGTTGGTGCAACCCGTTGT |
| | | CATGATCCGGAAGAAAAAGCATGTATTCTGCCGTTTTTAGGTGCAGGTCCGGATGG |
| | | TAGTCCGGAAAGCAATGGTTATTATACCGCAACCGATTATCAGGATATTCTGAGCC |
| | | ATGCAGCAGCACTGAATATTGAAGTTGTTCCGGAAATTGATATTCCGGGTCATGCA |
| | | CATGCAGCAATTAAAGCCATGGAAGCACGTTATGATCATTATGCAGCACAGGGTAA |
| | | TATGGCAGAAGCAAACAAATATCTGCTGACCGATTTTAATGATACCACGCAGTATCT |
| | | GAGCGTTCAGATGTTTACCGATAATGCGATTAATGTGTGTATGGAAAGCAGCTACA |
| | | ATTTTGTGGATGCAGTTGTTAGCGGTCTGGTTAGCCTGCATCAGGGTGTTCAGCCG |
| | | CTGAAAACCTTTCATTTTGGTGGTGATGAAATTGCCGGTGCCTGGATTAATAGTCC |
| | | GGCATGTCAGGATTTTATTGCCAATAACACCGATGGTGTGCATAGCGTTAGCGATC |
| | | TGAGCCGTTATTTTGTTGAACGTATTAGCGTTATCACCGCCAATTATGGTCTGGATA |
| | | TGGCAGGTTGGGAAGATGGTCTGATGCATGATGGTCAGGTTTATCCGCGTAGCCA |
| | | GATGGCAAATAATCAGCTGTGGGGTAATGCCTGGCAGAATATTTGGGAATGGGGT |
| | | GTTGCAGATCGTGCATATAATCTGGCCAACAACGATTATAAAGTGGTGTATAATCAT |
| | | GCGACCCACCTGTATTTTGATCATCCGTATGAACCGGATCCGAATGAACGCGGTTA |
| | | TTATTGGGCTCCGCGTTTTACAGATACCCGTAAAACCTTTGGTTTTATGCCGGATGA |
| | | TGTTTTTGCCAATGCAGATTTTACCCGTGCGGGTGCACCGATTACCAAAGCAGAAG |
| | | TTGTTGCATCAGCCGGTGTTAAAAAACTGCTGAAACCGGAAAATGTTCTGGGTCTG |
| | | CAGGGTAGCCTGTGGGCAGAAGCGGTGCGTACCGAAGATCAGTTTGAGGGTATGA |
| | | TTTTTCCGCGTGTTTTAGGTCTGGCCGAACGTGCATGGCATACCGCCACCTGGGA |
| | | AGCAAATGATAATGCAGGTATTGCACTGGATGAAACCGGTCGTAATGCCGATTATA |
| | | ACCATTTTGCAAATCTGCTGGGTCAGAAAGTTCTGCCGAAACTGGAACAGGCAGG |
| | | CATTGCATTTAATCTGCCGGTTCCAGGTGGTGTGATTGAAAATGGTGTGCTGCAGG |
| | | CAAATAGCACCTTTCCGGGTCTGACCATTGAATATAGCACCGATCAGGGCACCAGC |
| | | TGGCAGAGCTATGATCATCTGAATCCGCCTGCAGTTGCAGCGGGTGTGCAGCTGC |
| | | GTACCGTGAGCGGTCAGCGTACCAGCCGTGTTACCACCGTTAATATCGAA |

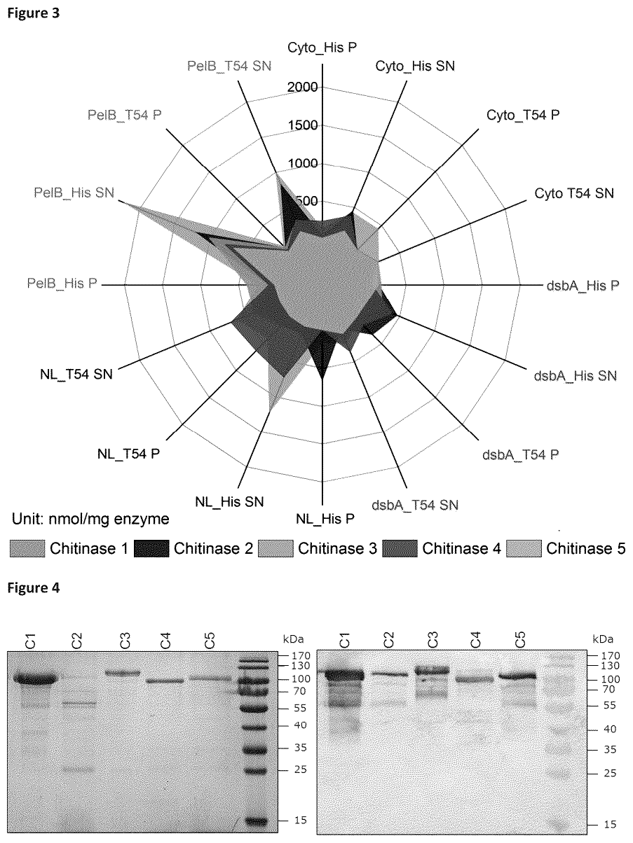

Integrity and correctness of the constructs was confirmed by colony PCR and Sanger-sequencing. All constructs were transformed into E. coli BL21 and expressed recombinantly. Expression levels of the target enzymes were determined densitometrically after immunoblot detection for cytosolic and secreted expression. Enzymatic activity was determined for all samples using a reducing end assay. Data from expression levels and activity were combined to obtain the specific enzyme activity (nmol reducing ends/mg enzyme) and it was revealed that variants comprising the PeIB signal peptide exhibit the overall highest activity (FIG. 3). Furthermore, it was confirmed that these enzymes are efficiently released into the culture supernatant, rendering cell lysis unnecessary for future production cycles. Based on these data, the enzyme versions with the PelB signal peptide were selected for further characterization studies.

### B. Purification of Recombinant Chitinases

A two-step purification of the selected recombinant enzymes with PelB signal peptide form the culture supernatant was carried out with an initial ammonium sulfate precipitation step and a sequential IMAC purification of the solubilized protein precipitate. Elution fractions were pooled and an SDS-PAGE and immunoblot was prepared loading the same volume for each sample (FIG. 4). Expression levels of the five chitinases varied largely, and C1 was detected in much greater abundance relative to the others. On the Coomassie stained SDS-PAGE gel, specific bands for C2 could not be detected after purification due to low expression levels, however immunoblot analysis revealed the presence and integrity of the enzyme. The observed molecular weight of all enzymes in SDS-PAGE gel and immunoblot was not in accordance with the expected theoretical molecular weight taking into account that a 6×His tag was added C-terminally to the enzymes and in general, bands for all enzymes were detected at a higher molecular weight. For all samples, unspecific bands could detected at ˜25 kDa and ˜50 kDa after IMAC purification, most likely representing host cell protein-contaminations. The amount of protein in the culture media, ammonium sulfate concentrates and the final dialyzed elution fractions was determined using the BCA assay and the same fractions were incubated with chitin powder for 16 h at 30° C. followed by the quantification of reducing sugars to measure specific chitinase activity (Table 4.2). Thereby it was confirmed that all target enzymes were successfully enriched during purification according to the specific activity.

TABLE 4.2
Summary of purification efficiency of recombinant chitinases C1-C5 against
chitin powder as a substrate. Total activity was assessed by the reducing
end assay and total protein content of the individual fractions was quantified
by the BCA assay. Both data were used to calculate the specific activity
in U/mg. Data represents means of three replicate experiments (n = 3).

| | | Total | Total | Specific | | |
|---|---|---|---|---|---|---|
| Enzyme/Purification | Volume | activity | protein | activity | Purification | Yield |
| fraction | [mL] | [U] | [mg] | [U/mg] | [−fold] | [%] |
| C1 | 800 | 127 | 1292 | 0.10 | 1.0 | 100 |
| Culture supernatant | | | | | | |
| C1 | 20 | 49 | 14 | 3.52 | 35.6 | 39.0 |
| Dialysed elution | | | | | | |
| C2 | 800 | 24 | 670 | 0.04 | 1.0 | 100 |
| Culture supernatant | | | | | | |
| C2 | 20 | 12 | 2.3 | 5.36 | 146.3 | 49.9 |
| Dialysed elution | | | | | | |
| C3 | 800 | 143 | 1104 | 0.13 | 1.0 | 100 |
| Culture supernatant | | | | | | |
| C3 | 20 | 53 | 6.5 | 8.13 | 62.9 | 36.9 |
| Dialysed elution | | | | | | |
| C4 | 800 | 57 | 791 | 0.07 | 1.0 | 100 |
| Culture supernatant | | | | | | |
| C4 | 20 | 19 | 3.1 | 6.07 | 84.1 | 33.1 |
| Dialysed elution | | | | | | |
| C5 | 800 | 25 | 965 | 0.03 | 1.0 | 100 |
| Culture supernatant | | | | | | |
| C5 | 20 | 12 | 1.9 | 6.67 | 258.9 | 50.0 |
| Dialysed elution | | | | | | |

### C. Characterization of Chitinolytic Enzymes

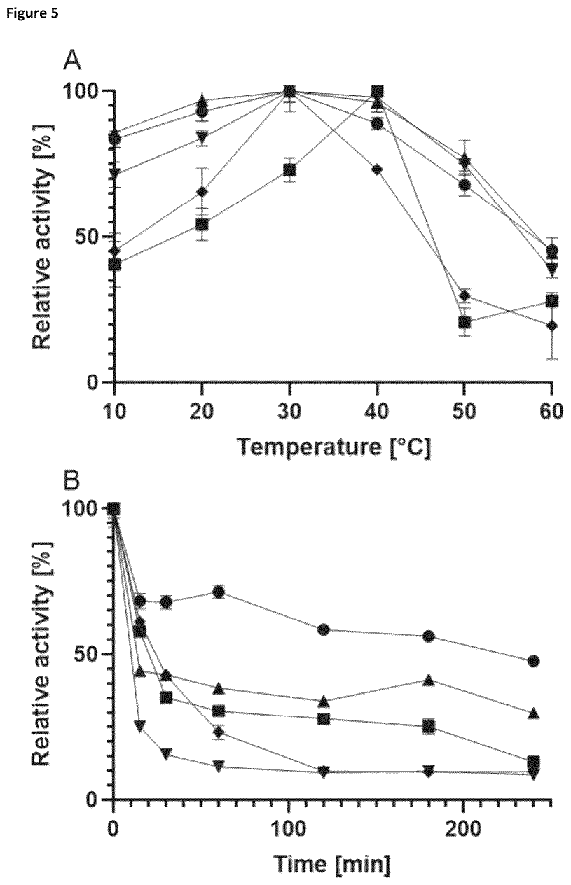

All enzymes were first characterized regarding their temperature and pH-optima as well as the optimum NaCl content as the enzymes were isolated from a sea-water microorganism. As the future intention is to integrate these novel enzymes in a degradation processes of natural chitin, all parameters were determined using chitin powder as a substrate instead of pre-treated analogs such as colloidal chitin, glycol chitin or artificial fluorogenic substrates. Investigating the effect of temperature showed that C1, 3 and 4 are highly active within a temperature range of 10-50° C., with their optimum temperature at 30° C. C2 and C5 are less tolerant and showed highest activity at 30° C. and 40° C., respectively, and lost more than 50% of activity at 50° C. and 60° C. (FIG. 5A). Next, enzyme temperature stability was investigated (FIG. 51). All enzymes showed reduced activity after 15 min incubation at 60° C. Enzyme C1 retained a 50% residual activity after 240 min incubation at 60° C., while the activity of the other chitinases dropped below 50% after 15 min and remained on that level.

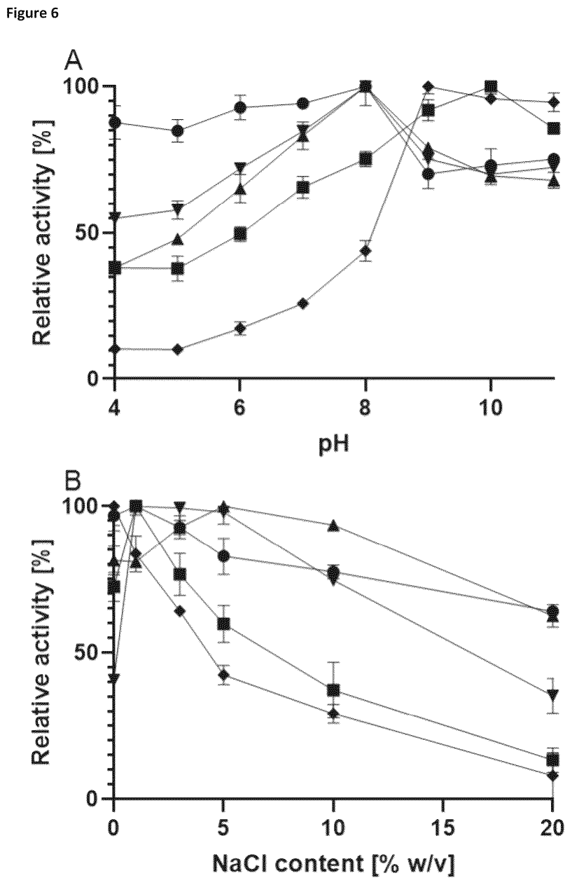

With regard to the pH, C1, 3 and 4 showed similar behavior and had the optimum at pH 8, while C2 and C5 showed optimum reaction conditions at pH 9 and pH 10, respectively (FIG. 6A).

Given the marine origin of the P. orarium strain, the effect of salt was also evaluated, testing the addition of NaCl to the standard buffer. NaCl amounts up to 5% (w/v) have a beneficial effect on enzyme activity for C1-C4 reflecting the average 3.5% salt content of the North Sea environment from which the bacterium was isolated (FIG. 6B). Increasing NaCl amounts up to 20% (w/v) reduced enzyme activity substantially to 10% for C2, 4 and 5, with C1 and C3 showing highest tolerance (65% activity). As expected, C5 that is not secreted by P. orarium showed the lowest tolerance to increasing salt contents reflecting the overall lower intracellular salt concentrations relative to salt contents in the North Sea. Enzyme optima for temperature, pH and salinity are summarized in Table 4.3.

TABLE 4.3
Optimum temperature, pH and salinity conditions for recombinant
chitinases (C1-C5) determined on chitin powder.

| | Temperature | | Salinity |
|---|---|---|---|
| Enzyme | [° C.] | pH | [% NaCl] |
| C1 | 30 | 8 | 1 |
| C2 | 40 | 10 | 1 |
| C3 | 30 | 8 | 5 |
| C4 | 30 | 8 | 1 |
| C5 | 30 | 9 | 0 |

The addition of different co-factors and chemicals was assessed to determine whether they stimulate or inhibit enzymatic activity and the effect was evaluated relative to the control in standard MAT buffer (33 mM 2-(N-morpholino) ethanesulfonic acid (MES), 33 mM sodium acetate, 33 mM Tris(hydroxymethyl) aminomethane (TRIS); pH 8.0). The selection of tested compounds at the respective concentrations was based on findings for different chitinases so far [Zarei et al., J. Microbiol., 2011. 42: p. 1017-1029.]. Incubation of 2 μM of enzymes with different metallic ions and chemical compounds revealed activity inhibitors (Table 4.4). All enzymes were inhibited the strongest by Zn2+ and Cu2+ and SDS (0.5% w/v) resulting in a relative loss of activity of up to 99%. C2 and 5 were furthermore substantially inhibited by all tested ions and no enhancers of enzymatic activity were identified. Inhibitory effects of Ni2+ and imidazole on enzymatic activity also underline the requirement for dialysis of the elution fractions after purification in order to remove residual Ni2+ and imidazole from the elution buffer.

Different chitinous substrates were tested to assess the substrate affinity of the novel chitinases relative to untreated chitin powder. Testing of different substrates revealed that chitin powder and colloidal chitin in general are degraded by all enzymes (Table 4.5). As expected, the conversion of colloidal chitin is more efficient compared to chitin powder as it provides an overall smaller particle size and thereby larger surface area. Chitosan powder (degree of acetylation: 15%) was also degraded efficiently and high chitosanase activity was observed especially for C1, C3, C4 and C5. Merely basal activity was detected when cellulose was utilized as a substrate for all enzymes.

### D. Enzyme Kinetics on Chitin Powder

Enzyme kinetics were determined using chitin powder as a substrate and the optimum enzyme to substrate ratio could be determined as well as Vmax and Km values. The concentrations of chitin powder varied

TABLE 4.4
Effect of selected metallic ions (1 mM) and chemical compounds at on chitinase activity
(C1-C5) relative to the standard MAT buffer control (100%). Data represents means
of three replicate experiments (n = 3).

| Metal ion/Chemical | [%] Residual | [%] Residual | [%] Residual | [%] Residual | [%] Residual |
|---|---|---|---|---|---|
| compound 1 mM | activity C1 | activity C2 | activity C3 | activity C4 | activity C5 |
| Control | 100.0 | 100.0 | 100.0 | 100.0 | 100.0 |
| Mg²⁺ | 99.8 ± 3.1 | 38.7 ± 1.7 | 68.8 ± 2.8 | 3.3 ± 0.2 | 25.8 ± 0.9 |
| Zn²⁺ | 30.5 ± 0.3 | 2.3 ± 0.4 | 21.7 ± 0.1 | 8.1 ± 0.7 | 0.6 ± 0.5 |
| Ca²⁺ | 92.7 ± 2.1 | 35.6 ± 1.7 | 94.7 ± 2.0 | 3.8 ± 0.4 | 21.3 ± 0.1 |
| EDTA | 68.9 ± 0.7 | 20.4 ± 1.6 | 80.8 ± 4.8 | 73.9 ± 4.1 | 28.0 ± 0.9 |
| Cu²⁺ | 11.1 ± 3.7 | 12.3 ± 0.9 | 16.9 ± 0.2 | 14.8 ± 3.4 | 3.9 ± 2.9 |
| Ni²⁺ | 76.6 ± 5.1 | 23.0 ± 0.7 | 64.8 ± 1.2 | 38.8 ± 1.7 | 5.6 ± 0.3 |
| K²⁺ | 100.1 ± 2.8 | 44.8 ± 2.3 | 72.6 ± 1.7 | 3.7 ± 0.2 | 28.6 ± 0.4 |
| Tween 20 (0.5% v/v) | 84.3 ± 4.1 | 4.2 ± 4.4 | 38.3 ± 5.4 | 63.6 ± 4.0 | 36.2 ± 1.3 |
| SDS (0.5% w/v] | 1.5 ± 0.2 | 21.6 ± 1.0 | 2.4 ± 1.9 | 33.2 ± 3.4 | 19.9 ± 0.3 |

TABLE 4.5
Relative (in % activity of chitin powder) activity testing of purified recombinant chitinases
(C1-C5) with chitin (powder), chitosan (medium molecular weight), colloidal chitin and
cellulose. Enzymes were incubated for 16 h at optimum conditions with 50 mg/ml of the
respective substrates and the reducing end assay was used for quantification of free sugars.
Data represents means of three replicate experiments (n = 3).

| | Chitin | Chitosan | Colloidal | Cellulose |
|---|---|---|---|---|
| Enzyme | [%] | [%] | chitin [%] | [%] |
| C1 | 100 | 117.1 ± 9.1 | 159.9 ± 4.8 | 14.3 ± 5.7 |
| C2 | 100 | 71.4 ± 6.7 | 191.4 ± 4.7 | 10.0 ± 1.1 |
| C3 | 100 | 113.4 ± 9.0 | 133.4 ± 6.5 | 9.4 ± 0.9 |
| C4 | 100 | 141.3 ± 5.5 | 117.7 ± 6.3 | 10.4 ± 0.9 |
| C5 | 100 | 117.8 ± 1.7 | 164.1 ± 2.8 | 11.2 ± 1.1 |

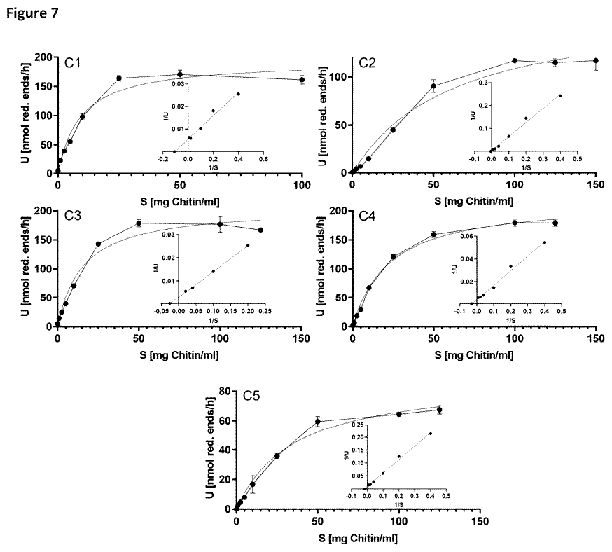

from 0 mg/ml up to 150 mg/ml while amount of enzyme was kept constant (2 μM). The initial velocities (V0) versus substrate concentration ([S]) were plotted as Lineweaver-Burk and Michaelis-Menten curves using GraphPad software (FIG. 7).

Maximum reaction velocities (Vmax) and Michaelis-Menten constants (Km) were determined using a non-linear fit Michaelis-Menten model (Table 4.6). Thereby, optimum enzyme-substrate ratios were determined that would allow a maximization of substrate conversion rates. Furthermore, the Km indicated the reciprocal enzyme-substrate affinity for C1-C5. Higher Km values reflect an overall lower affinity and thus reduced conversion efficiencies. The data indicated that C1 and C3 have the highest affinity to insoluble chitin while maintaining an overall high Vmax. In comparison, higher Km values for C2 and C5 reflected an overall lower affinity to insoluble chitin.

TABLE 4.6
Enzyme and substrate specific kinetic parameters Vₘₐₓ
(maximum reaction velocity) and Kₘ (enzyme-substrate affinity) for
recombinant chitinases determined on insoluble chitin powder using a
non-linear fit model.

| Enzyme | Vₘₐₓ [U/ml] | Kₘ [mg/ml] |
|---|---|---|
| C1 | 197.7 | 9.1 |
| C2 | 175.4 | 61.8 |
| C3 | 206.2 | 15.2 |
| C4 | 218.6 | 22.2 |
| C5 | 89.5 | 36.0 |

### E. Analysis of Hydrolysis Products

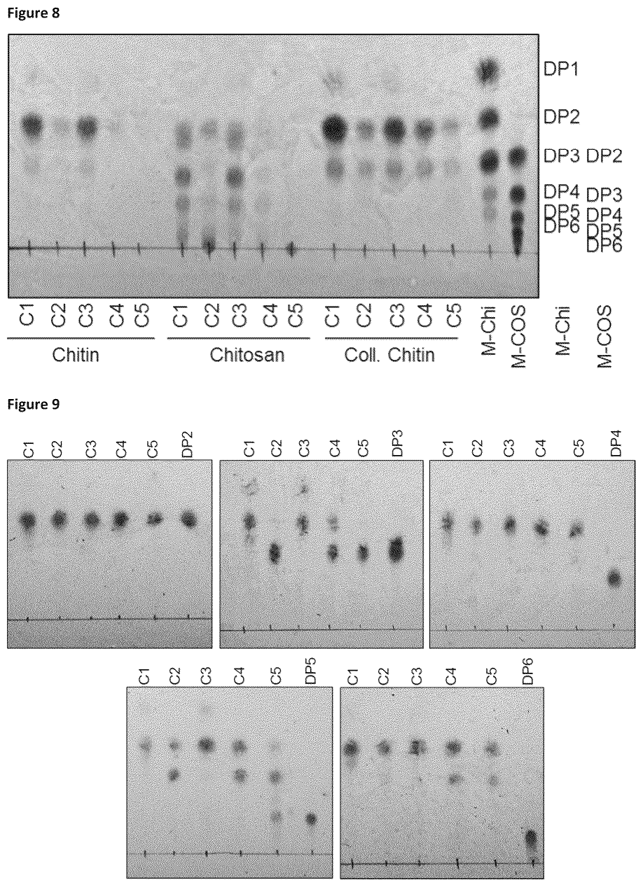

The five recombinant chitinases were tested with different substrates at optimum reaction conditions, and hydrolysis products were identified using TLC (FIG. 8). Depending on the substrates, the different enzymes yielded different product ranges. Colloidal chitin and chitin powder were mainly degraded to dimeric, trimeric and

monomeric oligomers. Chitosan was degraded to a crude mixture of multiple oligomers ranging from dimers to pentamers and even larger undefined yet water-soluble fragments that migrated slightly from the starting point. Further analysis of the cleavage patterns was carried out using chitin standard molecules of DP2-DP6 and incubating them at 4 mg/ml concentration with the individual enzymes (FIG. 9).

Dimeric chitin molecules were not converted by any of the enzymes. Surprisingly, as the Pfam search revealed that C5 contains a chitobiase domain supposed to cleave dimers to yield GlcNAc, no exochitinase activity was observed. Solely C1, 3 and 4 cleaved chitin trimers to dimers and monomers, with C2 and C5 showing no activity on trimers. All chitinases degraded chitin tetramers to dimers as a single product. Chitin pentamers were converted to dimers and monomers by C1 and C3 and to trimers and dimers by C2,4 and 5. Chitin hexamers were degraded to dimers and trimers. Although all chitinases are different in their structure and present different glycosyl hydrolase domains hydrolysis products in general were similar.

### Discussion

The recombinant overexpression of chitinolytic enzymes attracted great attention in the last few decades, as chitinases are either utilized directly as antifungal agents or alternatively to elucidate novel mechanisms to enzymatically degrade chitin into functional oligomers. Chitinolytic bacteria utilize chitin for their metabolism and therefore represent a great source for endo-, and exo-chitinases essential to convert chitin into available GlcNAc. Five different putative chitinolytic enzymes were discovered within the genome of P. orarium that are utilized for a complete degradation of insoluble chitin to GlcNAC and GlcNAC2. Pfam searches revealed homologies to glycosyl hydrolase family 18,19 and 20 characteristic for endo, and exo-chitinase activity. Natural signal peptides were identified for C1-C4 indicating that the respective enzymes are secreted by P. orarium in order to carry out an extracellular degradation. The sequence for C5 did not contain any specific signal peptide signifying that the enzyme remains intercellular for the further processing of assimilated dimeric chitin. This is further supported by the presence of a chitobiase domain.

In order to carry out an individual and adequate characterization of the identified chitinases, singular enzymes must first be isolated and purified to remove potential interferences by residual medium constituents as well as similar chitinolytic enzymes. The isolation and purification of single chitinases from the complex P. orarium supernatant was investigated earlier using ion-exchange chromatographic approaches. However, results revealed that a complete isolation of single enzymes could not be achieved (data not shown). Therefore, a recombinant approach was followed to produce all chitinases separately using E. coli BL21.

The golden gate cloning approach was used to introduce different variants of the genes-of-interest into a bacterial expression plasmid. The natural signal peptide and the PelB signal peptide were tested both for a secretion by E. coli BL21 thereby exploiting the oxidative milieu in the periplasmatic space that promotes correct protein folding. Similarly, an N-terminal fusion of the target enzymes to the dsbA protein was investigated to enhance the formation of disulfide bonds for the generation of active enzymes. Two different purification tags (6×His tag and Tag54/6×His tag) were tested to investigate whether their introduction will interfere with protein expression and activity. For all five genes, constructs with a N-terminal PelB signal peptide for secretion and a C-terminal 6×His tag for purification encoded enzymes with the highest specific activity and were selected for further research. An initial experiment of enzyme purification from the culture supernatant injected directly on the Ni2+ charged Chelating Sepharose FF column, revealed that the spent medium contained an unknown metal-complexing constituent causing ion leakage that resulted in a significant loss of binding capacity (data not shown). Thus, it became necessary to carry out an ammonia sulfate precipitation step before IMAC purification to remove interfering substances by buffer exchange to PBS (pH 8.0).

Typically, chitin powder is not utilized to determine chitinase parameters, as it is highly crystalline, less defined concerning particle size and DA, has a relatively low specific surface area and the initial molecular mass is difficult to calculate. However, in comparison to more artificial and pre-treated substrates such as glycol chitin, colloidal chitin or synthetic fluorogenic substrates that are often used to determine enzyme properties, powdery chitin reflects realistic process parameters for later applications of the enzymes in degradation processes.

Enzyme characterization studies identified optimum conditions with regards to temperature, pH and NaCl content, functional substrates as well as effects of putative co-factors for all five enzymes. Furthermore, the determination of enzyme kinetics indicate optimum enzyme to substrate ratios. Optimum temperatures for C1-C5 (30-40° C.; FIG. 5A) are overall lower than those reported in the literature for bacterial chitinases, as these enzymes are predominantly reported as thermophilic or thermo-tolerant enzymes with optima in the 40-60° C. range [Krolicka et al., J Agric Food Chem, 2018. 66(7): p. 1658-1669.; Menghiu et al., Protein Expr Purif, 2019. 154: p. 25-32; Pechsrichuang et al., Bioresour Technol, 2013. 127: p. 407-14.; Zhang et al., Biotechnol. Biofuels, 2018. 11(1): p. 179.]. Typically in industrial processes high temperatures are favored as solubility of hydrophobic compounds and the overall biodegradation process can be enhanced. Thus, thermophilic enzymes are implemented in such processes as the can withstand higher temperatures while maintaining activity over a longer period of time. Such thermophilic enzymes are typically isolated from thermophilic bacteria and fungi or modified synthetically by protein engineering. The novel chitinases of P. orarium showed to be highly active at more moderate temperatures and can maintain up to 50% activity at 60° C. The overall lower optima can be linked to the natural sea-water habitat of P. orarium with an average temperature of 5-25° C. Overall for an industrial chitin degradation process these properties could become beneficial as the enzymes are highly active on chitin powder that would not require elevated temperature thus reducing the total energy costs and process efforts.

Optimum pH conditions were determined to be between pH 8 and 10 for the five novel enzymes (FIG. 6A) reflecting adaptation to the slightly basic conditions in sea water (pH 7.5-8.4) the P. orarium strain was isolated from. Literature data reported various pH optima for different chitinolytic enzymes ranging from pH 4-8, depending on the environment the enzymes were originally isolated from. The addition of salt (1-5% (w/v) NaCl) resulted in increased enzyme activity (FIG. 6B) reflecting adaptation of the chitinases to marine conditions. C1, 3 and 4 maintained a relative activity of up 75% at 20% (w/v) salt content. Although several chitinases were also isolated from marine sources before and salinity is an essential parameter as the enzymes may have evolved to possess higher activity at a certain salt content, only limited data is published in literature on the determination of optimum salinity for chitinases. However, the findings for C1-C5 are in accordance with the data reported on preservation of activity at salinity up to 20% (w/v). As the mild temperature optima of the chitinases (30°-40° C.) could promote bacterial contaminations in non-sterile reaction conditions, additions of salt to the reaction mix could minimize the risk of contaminations.

Several co-factors were tested to elucidate whether enzyme activity can be improved and to identify potential reaction inhibitors. Co-factor analysis revealed that all tested metal ions (Mg2+, Zn2+, Ca2+, Cu2+, Ni2+, K+), chemicals (EDTA, Imidazole) and detergents (SDS, Tween 20, Triton X100) can be considered activity repressors and no activators were identified. C1 exhibits relatively high tolerance to Mg2+, Ca2+, K+ions, Tween 20, Triton X100 and Imidazole with a minimal residual activity of 84.3% relative to the control. On the other side C2, C4 and C5 were strongly inhibited by almost all compounds with most residual activities reduced below 40% of initial activity (Table 4.4). These results may furthermore prove that the studied chitinases are not metalloenyzmes, as also reported for various bacterial chitinases [Zarei et al., Microbiol., 2011. 42: p. 1017-1029.].

Enzyme kinetics were determined using chitin powder as a substrate. Substrate properties can have major impact on enzyme kinetics and colloidal chitin and untreated chitin powder differ largely in terms of their overall material properties: 1) The particle size and crystallinity of colloidal chitin is substantially lower than chitin powder 2) the surface area of colloidal chitin is higher resulting in increased enzyme accessibility. These different material parameters greatly affect the overall enzymatic activity of the chitinases and thereby conversion rates are increased substantially for colloidal chitin. However, the utilized substrate for enzyme characterizations should always be selected based on the intended future application to assess enzyme parameters under accurate process conditions. The novel chitinases will be implemented in a fully enzymatic conversion process of chitin to COS and harsh chemical substrate pre-treatment methods should be minimized or even eliminated completely. Therefore, the use of chitin powder helped to assess optimum enzyme—substrate ratios in order to maximize reaction rates.

Digestion experiments revealed that mixtures of soluble oligomers are produced ranging mainly from monomeric GlcNAG to GlcNAG3 with chitin as a substrate, and larger oligomers (≥pentamers) were observed when chitosan was degraded. Further analysis on the cleavage mechanisms revealed that C1 and C3 did exhibit exo-chitinase activity independent on oligomeric or polymeric chitin substrates (FIG. 8, FIG. 9) as monomeric GlcNAC was detected by TLC. C2, 4 and 5 yielded similar product patterns when incubated with insoluble substrates. As it was further revealed after digestion of standard molecules, C2, 4 and 5 did not yield GlcNAc, thereby exhibiting endo-chitinase activity. The presented data underline that the novel chitinases are all suited for the degradation of powdery chitin without any further harsh chemical pre-treatment.

Enzymatic degradation reactions will be further investigated in multiple aspects: 1) stimulate the production of single oligomers with a specific DP and reduce the amount of undesired oligomers and GlcNAc; 2) produce oligormers with DP>3; 3) maximize the overall conversion rates and product yields. Therefore, synergistic effects of multi-enzyme reactions will be assessed and process optimization will be carried out using the design-of-experiments approach.

### Conclusions

The genes for five novel potential chitinases were identified from genome data of the novel bacterial strain P. orarium and successfully expressed and purified from E. coli BL21. The novel enzymes are relatively large compared to current reported bacterial chitinases and also show lower temperature and pH optima with chitin powder as substrate, as well as a substantial tolerance to salinity. The lower temperature optimum at 30° C. will be beneficial especially when considering reactions at larger scales as it involves less effort and expenses when establishing and maintaining the process. The high salt tolerance could furthermore be exploited to establish non-sterile degradation reactions with higher salt content potentially inhibiting bacterial contaminations. It was also discovered that all chitinases convert chitin and colloidal chitin to mainly dimeric, trimeric and monomeric compounds and chitosan to a cruder mixture of different COS. Future analyses will focus on the implementation of the different chitinases in in vitro cocktails to maximize the conversion rates and to investigate whether different degradation products with regards to the DP can be obtained by multi-enzyme reactions.

### Experimental Section

### Materials

All chemicals used in the study were of highest purity and obtained from Carl-Roth (Germany). Chelating Sepharose FF was purchased from GE Healthcare (Sweden). All utilized buffers were prepared freshly in demineralized water.

### Bacterial Strains and Plasmids

The novel marine chitinolytic P. orarium strain was isolated from seawater samples from Oostende, Belgium. The E. coli strain DH5a dam-/dcm- (New England Biolabs, Ipswich, USA) was grown in lysogeny-broth (LB) medium. The E. coli BL21 (DE3) (New England Biolabs) strain was grown in terrific-broth (TB) medium. If needed, both media were supplemented with appropriate concentrations of ampicillin (100 μg/ml) or kanamycin (50 μg/ml).

### Next Generation Sequencing and Gene Mining

Genomic DNA was extracted from a liquid culture of P. orarium using the “NucleoBond AxG500” kit (Machery Nagel) according to the manufacturers instructions. The DNA sample was further processed for the de novo whole genome sequencing using the “Ion Xpress Plus gDNA Fragment Library Preparation” kit (Machery Nagel) and ion

torrent sequencing was carried out by the Fraunhofer Institute for Molecular Biology and Applied Ecology IME, Aachen (Ion Torrent Personal Genome Machine PGM, Thermo Fisher Scientific). The DNA-STAR assembly method lead to 60 contigs that were assembled onto scaffolds (10-fold coverage). The protein family database (Pfam; https://pfam.xfam.org) was used to identify homologous polysaccharide binding domains and chitinases active sites in the scaffolds reflecting the presence of putative chitinases. The SignalP 4.1 server was used to obtain potential signal peptides responsible for the secretion of the enzymes (http://www.cbs.dtu.dk/services/SignalP-4.1).

### Cloning of Chitinase Genes and Sequencing

The golden gate cloning technology was employed to clone the target genes into the pET39b(+) expression vector resulting in multiple constructs for each gene-of-interest. Golden gate cloning enables a simultaneous directed ligation of multiple genetic fragments or bricks into a vector backbone using type IIs restriction enzymes

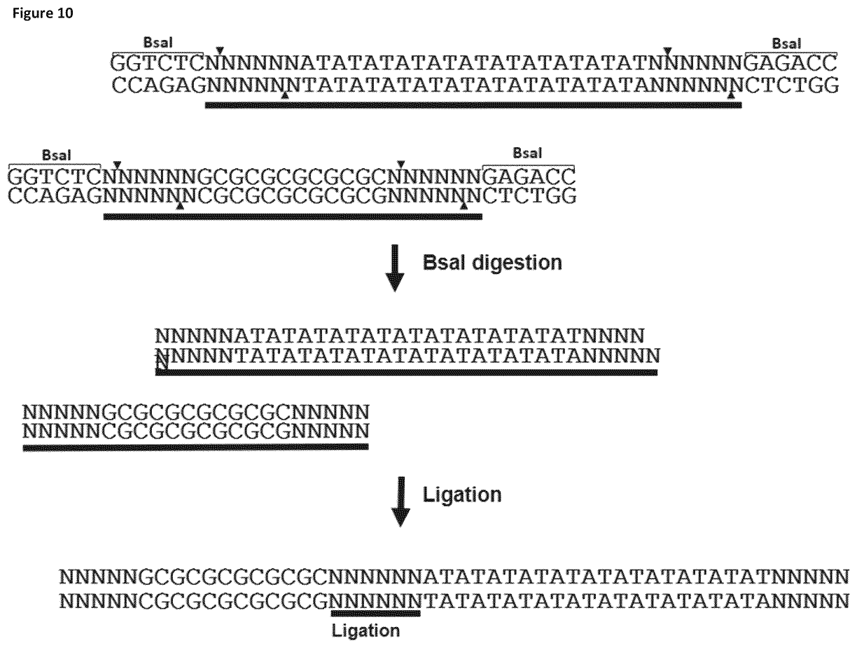

that cut outside their recognition site [El-Shemy et al., PLoS ONE, 2008. 3(11): p. e3647; Engler et al., PLoS One, 2009. 4(5): p. e5553.]. A preceding in silico design of specific cleavage sites and overhangs allows a high-throughput assembly of constructs from multiple gene-bricks in a one-pot restriction and ligation step. The general principle of golden gate cloning is displayed in FIG. 10. The sequences in FIG. 10 in order of appearance are included as SEQ ID NOs: 13-17 in the sequence listing.

Multiple genetic elements that allow a successful integration and functional expression of the novel enzymes by E. coli BL21(DE3) were assessed. Natural signal peptides identified after the Pfam search and the commonly used PeIB signal peptide from Erwinia carotovora were tested N-terminally for secretion of the target enzymes into the culture medium. A fusion to a bacterial periplasmatic oxidoreductase (dsbA) acting as a folding enhancer to oxidize disulfide bonds as well as a variant for cytosolic expression were tested. A 6×His tag or alternatively a tag546×His combi-tag were both introduced C-terminally as purification tags. Three distinct entry vectors were designed from the pET39b(+) to assemble the constructs in the correct orientation (Table 4.7): pGR_SigP for secretion of the target enzymes; pGR_dsbA for fusion to the dsbA protein and pGR_cyto for cytosolic expression.

All genetic bricks and vector backbones to assemble the final constructs were modified and synthesized (Thermo Fisher Scientific, Waltham, USA) with flanking BsaI recognition sites (5′ . . . GGTCTC(N)1▾ . . . 3′ and 3′ . . . CCAGAG(N)5▴ . . . 5) with the proper overhangs in the correct orientation to ensure the directed assembly of the constructs (Table 4.1). Cloning was carried out using the golden gate assembly mix according to the manufacturer's instructions (New England Biolabs). Integration and correctness of inserts was verified by colony PCR and Sanger-sequencing using T7 primers T7F(5′AAATTAATACGACTCACTATAGGG3′, SEQ ID NO: 18) and T7R (5′ATGCTAGTTATTGCTCAGCGG3′, SEQ ID NO: 19) flanking the cloned constructs.

Expression of Recombinant Chitinases in E. coli BL21 (DE3)

TABLE 4.7
Overhang design of genetic bricks and vectors
utilized in golden gate cloning. The 5′ and 3′
single stranded overhangs are generated after
digestion with the BsaI restriction enzyme and
allow a directed assembly of the constructs.

| Genetic element | 5′ overhang | 3′ overhang |
|---|---|---|
| pGR_SigP | TGAG | TGTA |
| pGR_dsbA | TGAG | CCGG |
| pGR_cyto | TGAG | CCGG |
| Natural signal peptide | ACAT | CCGG |
| PelB signal peptide | ACAT | CCGG |
| Gene-of-interest | GGCC | GCTT |
| 6xHis-tag | CGAA | ACTC |
| Tag54/6xHis-tag | CGAA | ACTC |

For the expression analysis of the chitinase constructs, freshly heat-shock transformed E. coli BL21 (DE3) cells were used to inoculate an overnight starter culture (180 rpm, 37° C.) in 20 mL TB medium supplemented with 50 μg/ml kanamycin. The starter culture was used to inoculate the main culture 1:100 in 25 ml TB-medium using 100 mL Ultra Yield™ flasks (Thomson Instrument Company, Oceanside, USA). Cultivation was carried out at 37° C. in orbital shakers at a shaking velocity of 200 rpm. After 4 h of cultivation (OD600 nm=4), cells were induced in the middle of the log-phase by addition of 1 mM isopropyl-s-D-thio-galactopyranoside (IPTG) to the culture broth and temperature was decreased to 28° C. After reaching a total cultivation time of 18 h, cells and supernatant were separated by centrifugation (8000×g, 30 min). For cell lysis, an aliquot of the pellet was resuspended in BugBuster® master mix (Merck Millipore, USA) and incubated at RT for 2 h in an overhead shaker (60 rpm). Cell debris were separated by centrifugation (8000×g, 2 min) and the resulting lysis supernatant as well as culture supernatant were subjected to densitometric analysis of immunoblot images and enzyme activity assay. Selected optimal enzyme candidates were produced at larger scale using 800 ml TB-medium and 2.5 L Ultra Yield™ flasks. Cultivation of starter cultures, induction and harvesting procedures were analogous to those above mentioned for small-scale expression.

Purification of Chitinases from Culture Supernatant

The protocol for the purification of 6×His tagged chitinases was adapted from “Affinity Chromatography Vol. 2: Tagged Proteins” (GE Healthcare) and consisted of two steps:
Step 1. Ammonium sulfate precipitation
Solid ammonium sulfate was slowly added to the culture supernatant to a final concentration of 70% and was stirred for 2 h at room temperature. The precipitate was collected by centrifugation (8000×g, 30 min) and dissolved in 0.1× volumes of PBS (pH 8.0) relative to the starting volume. Concentrated samples were centrifuged (8000×g, 10 min) and filtrated (0.45 μM) in order to remove any insoluble particles.
Step 2. Immobilized metal affinity chromatography (IMAC)
Samples from step 1 were applied to a Chelating Sepharose FF (GE Healthcare, Sweden) resin [column volume (CV) 5 ml] charged with 0.2 M NiSO4. The column was connected to an ÄKTA pure 25 (GE Healthcare), equilibration was carried out using 3 CV PBS (pH 8.0) and samples were injected directly on the column using a sample pump (2 ml min-1). The column was washed with equilibration buffer until the UV280 nm signal reached <30 mAU and weakly bound unspecific protein was eluted first from the column using 50 mM imidazole in PBS (pH 8.0) followed by a second elution step with 250 mM imidazole in PBS (pH 8.0). All steps were carried out at a constant flow rate of 2 ml min-1. Flow-through-, wash- and elution-fractions were collected using a fraction collector and elution fractions containing chitinases were pooled and dialyzed against a mixture of 33 mM MES, 33 mM CAPS, 33 mM TRIS (MAT-buffer; pH 8).

### SDS-PAGE, Immunoblot Analysis and Enzyme Assay

Protein analysis was carried out by SDS-PAGE using 12% separating gels and protein bands were visualized by staining with Coomassie Brilliant Blue R-250. For immunoblot analysis, samples from SDS-PAGE gel were transferred to a nitrocellulose membrane by tank-blotting; detection was carried out with a primary mouse monoclonal 6×His-tag antibody [0.2 μg/ml] (Thermo Fisher Scientific) and sequential band visualization with the secondary anti-mouse antibody alkaline phosphatase conjugate [0.2 μg/ml] (Thermo Fisher Scientific) using NBT/BCIP as a substrate. Samples were quantified densitometrically using the AIDA 5 software (Raytest IsotopenmessgerAte GmbH) relative to the 6×His tagged protein K12v105 (Fraunhofer IME). Quantification of purified proteins was carried out via the bicinchoniic acid assay (BCA) using bovine serum albumin as standard for calibration [Smith et al., Anal. Biochem., 1985. 150: p. 76-85.].

For the determination of enzymatic activity of recombinant chitinases, a reducing end assay was carried out [Svein et al., Carbohydr Polym, 2004. 56(1): p. 35-39.]. Fractions containing chitinases were incubated with 5% (w/v) chitin powder extracted from shrimp shells (˜400.000 g/mol; Carl Roth) in MAT buffer for 2 h in an overhead shaker at ambient temperature (standard conditions). Samples were centrifuged at 13000×g for 2 min and 40 μl of the supernatant was mixed with 40 μl 0.5 M NaOH and 40 μL of a reagent containing 1.5 mg/ml 3-Methyl-2-benzothiazolinonhydrazon and 0.75 mg/ml Dithiothreitol. Samples were incubated at 80° C. for 15 min and then thoroughly mixed with 80 μl of 0.5% (w/v) FeNH4(SO4)2)×12H2O, 0.5% (w/v) sulfamic acid and 0.25 M HCl. After cooling down to room temperature, 100 μl of the samples were measured at 620 nm. A calibration curve of N-acetyl-glucosamine was constructed freshly for each measuring cycle. One unit [U] was defined as the amount of enzyme required to release 1 μmol of reducing sugars per hour.

### Preparation of Homogenous Chitin Powder

Chitin from shrimp shells (˜400.000 g/mol; Carl Roth) was mechanically pre-treated using a GyroGrinder (Fritsch GmbH, Germany) at 6000 rpm and thereby converted to a homogeneous powder of 80 μM average particle diameter. The powder was used without any further treatment as a substrate for all chitinolytic degradation experiments.

### Preparation of Colloidal Chitin

The preparation of colloidal chitin was similar to the method described by Murthy and Bleakley with some minor differences [Murthy et al., Microbiol., 2012. 10(2): p. 1-5.]. Chitin powder (Carl Roth) was dissolved in concentrated hydrochloric acid (HCl) (5 g in 100 mL) and stirred at 4° C. for 24 h, after which the mixture was centrifuged at 4000×g for 15 min at 4° C. The precipitate was washed to neutral pH with distilled water and stored at 4° C.

### Characterization of Recombinant Chitinases

Enzyme samples were incubated at different temperature (10-60° C., 10° C. increment) and pH (4-11, increment 1) using different buffer systems (Acetate, Tris-HCl, MES, total molar concentration 100 mM), as well as NaCl content (0, 1. 2.5, 5, 10 and 20% w/v) to determine optimum reaction conditions. All experiments were carried out at 1.0 ml scale using 2 μM of the respective enzymes and a 5% (wv) suspension of chitin powder extracted from shrimp shells (˜400.000 g/mol; Carl, Roth) in 100 mM MAT buffer. For the determination of the substrate specificity, chitin powder (5% w/v), glycol chitin (10% v/v), colloidal chitin (5% w/v), chitosan [DA 15-25%, medium molecular weight, Sigma-Aldrich] (5% w/v) and microcrystalline cellulose powder (Sigma Aldrich) (5% w/v) were investigated, respectively. The effect of several potential co-factors on activity was elucidated at a concentration of 1 mM. Additionally, the effect of detergents [SDS 0.5% (w/v), Tween-20 and Triton X-100 both at 0.5% (v/v)] and chemicals (Imidazole 100 mM, EDTA 1 mM) on enzymatic activity was assessed. Samples were incubated in a thermomixer for 16 h at 900 rpm. After incubation, samples were analyzed using the enzyme assay. Thermostability was assessed after incubation of recombinant chitinases at 60° C. for different durations followed by the quantification of residual enzyme activity under standard assay conditions. Enzyme kinetics of recombinant chitinases were assessed by determination of Km and Vmax by Lineweaver-Burke representation of the Michaelis-Menten model preceding an incubation of constant amounts of enzyme (2 μM) with 0-150 mg/ml chitin and sequential quantification of reducing sugars.

### Product Analysis by Thin-Layer-Chromatography

Hydrolysis products were analyzed using 10×10 cm TLC Silica gel 60 F254 plates (Merck, Darmstadt, Germany) and a mixture of butanol:methanol:25% ammonia:H₂O (5:4:2:1) as mobile phase. A total sample volume of 0.5 μl was applied in 0.25 μl spots. Chitin standard sugars (Megazyme, Chicago, USA) (5 mg/ml) were applied to the plate (0.5 μL in 0.25 μL spots) as size standard for the determination of the DP. After separation, the plate was air-dried and the developing solution (200 ml Acetone, 30 ml phosphoric acid (85%), 4 ml aniline, 4 g diphenylamine) was sprayed on the plate followed by spot visualization at 300° C. using a heat gun.

### Example 3

### Introduction

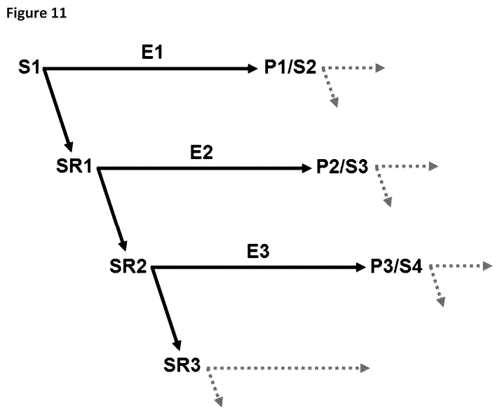

This example describes the development of a fully enzymatic depolymerization process for the controlled degradation of chitin powder using the chitinases produced recombinantiy and characterized in Example 2. Because the product spectrum of individual enzyme reactions concerning the degree of polymerization (DP) did not show major differences, synergistic effects of various chitinase combinations were investigated. Mixing enzymes with different hydrolytic domains can yield different products compared to single enzyme reactions, as cascade enzyme-substrate interactions and feed-back effects can occur. In particular, products of one enzymatic reaction can serve as substrates for different enzymes, thereby yielding final products with different properties (FIG. 11). Further potential benefits from multi-enzyme processes are synergistic effects that could result in higher overall products yields due to the shift of the reaction equilibrium along with a reduction of product inhibitions.

Although enzymatic reactions are superior to chemical reactions in terms of controllability and selectivity, major drawbacks are relatively low conversion rates due to product inhibitions as well as limited substrate availability. So far, no fully cascade multi-enzyme conversion process from insoluble chitin to defined COS has been reported. Still, it is desired to eliminate chemical pre-treatment and degradation steps entirely and to establish fully enzymatic processes. Due to the diverse nature of chitinolytic enzymes with different catalytic mechanisms and substrate specificities, intensification of the process can be achieved by modeling multi-enzyme reactions. Process analysis and optimization are essential methodologies to assess complex multi-factor procedures such as mixed enzyme reactions. A common method to investigate and optimize processes is to sequentially vary one putative influential factor and keep all other factors constant. This one-factor-at-a-time (OFAT) approach, however, conceals factor interactions and cannot expose synergistic interdependencies of two or more factors on a given output, thereby missing the true optimum. In contrast, the design-of-experiments (DoE) approach is a mathematical tool to carry out a reduced number of experiments in a systematic way to obtain meaningful information on the factor influences and factor interactions. The underlying statistical analysis allows to 1) assess all significant factors and factor interactions simultaneously 2) identify optimum factor combinations 3) extrapolate and interpolate factor combinations to achieve a desired output [Buyel and Fischer, J Vis Exp, 2014(83): p. 1-17; Rasche et al., Sci Rep, 2016. 6: p. 1-6; Vasilev et al., PLoS One, 2014. 9(8): p. 1-7; Kumaret al., Microbiol. Biotech. Res., 2011. 1(2): p. 33-53].

In this example, separate special cubic mixture designs are generated for chitinases. Each enzyme is considered a separate factor in a mixture and the effect on total conversion rate and product properties is studied. The I-optimal design type is selected for both mixture designs as the average variance of predictions is minimized resulting in more precise predictions of optimal mixtures. Optimized mixtures for chitinases were validated experimentally and implemented in depolymerization and deacetylation reactions for the conversion of chitin powder to partially deacetylated COS.

### Results

All five chitinases implemented in the DoE originated from a novel marine Photobacterium strain expressing these enzymes to carry out a conversion of crystalline chitin to monomeric and dimeric polysaccharides. All chitinases were produced recombinantly using E. coli BL21 (DE3) and characterized regarding optimum reaction conditions and product properties in Example 2. As so far only singular enzyme reactions were carried out, the DoE approach was used to 1) determine ideal enzyme combinations to maximize product rates 2) identify possible enzyme combinations to alter product distribution in terms of DP. To assess the effects of the individual factors (chitinase 1-5; C1-5) as well as two- and three-factor interactions, an I-optimal mixture design was set up and a special cubic model was implemented for evaluation. Enzymes were dialyzed and pre-diluted using MAT buffer (pH8) in order to add equal volumes recommended by the mixture design plan. Chitin and enzymes were used from the same batches to ensure consistent experimental conditions. The amount of released reducing sugars from the enzymatic degradation was quantified by the reducing end assay and used as a primary response.

Analysis of variance (ANOVA) indicated that all main factors had highly significant impact on the degradation of the substrate. Furthermore, highly significant two factor and three factor interactions between the

TABLE 6.1
Significant factors and factor interactions for the chitinase mixture
design. A reduced cubic model was used to analyze the response data
(released reducing sugars in nmol/ml × h). Significant main factors are
A (chitinase 1), B (chitinase 2). C (chitinase 3), D (chitinase 4) and E
(chitinase 5). Significant factor interdependencies were preselected by an
automated backward selection with a p-value threshold of 0.05. Factors
that were required to maintain model hierarchy were not excluded.

| Factor | F-value | P-value |
|---|---|---|
| Model | 168.97 | <0.0001 |
| Linear mixture | 585.86 | <0.0001 |
| AB | 12.12 | 0.0016 |
| AD | 87.53 | <0.0001 |
| AE | 162.03 | <0.0001 |
| BC | 21.41 | <0.0001 |
| CD | 88.40 | <0.0001 |
| CE | 32.21 | <0.0001 |
| ABE | 17.98 | 0.0002 |
| BCD | 16.15 | 0.0004 |
| BCE | 20.65 | <0.0001 |

*significant main factors: A, B, C, D and E.

TABLE 6.2
Model parameters to confirm significance for chitinase mixture design.

| Parameter | Value |
|---|---|
| R² | 0.9906 |
| Adjusted R² | 0.9847 |
| Predicted R² | 0.9381 |
| Lack of Fit | 0.7747 |

main factors were identified (Table 6.1). Significance of the model was confirmed by the not-significant lack-of-fit test and the predicted R2 value was in reasonable agreement with the adjusted R2 value (Table 6.2).

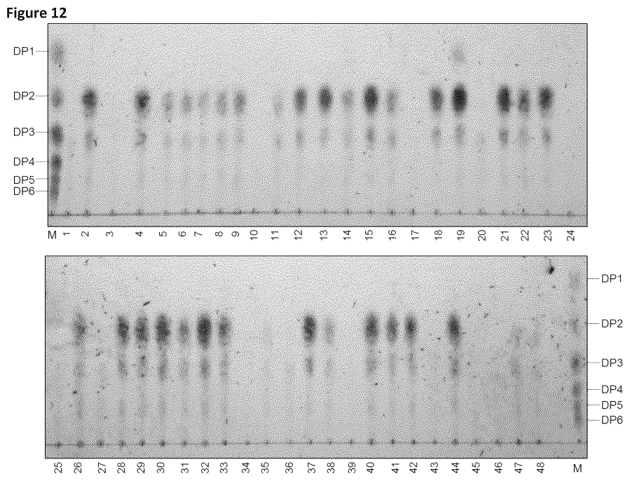

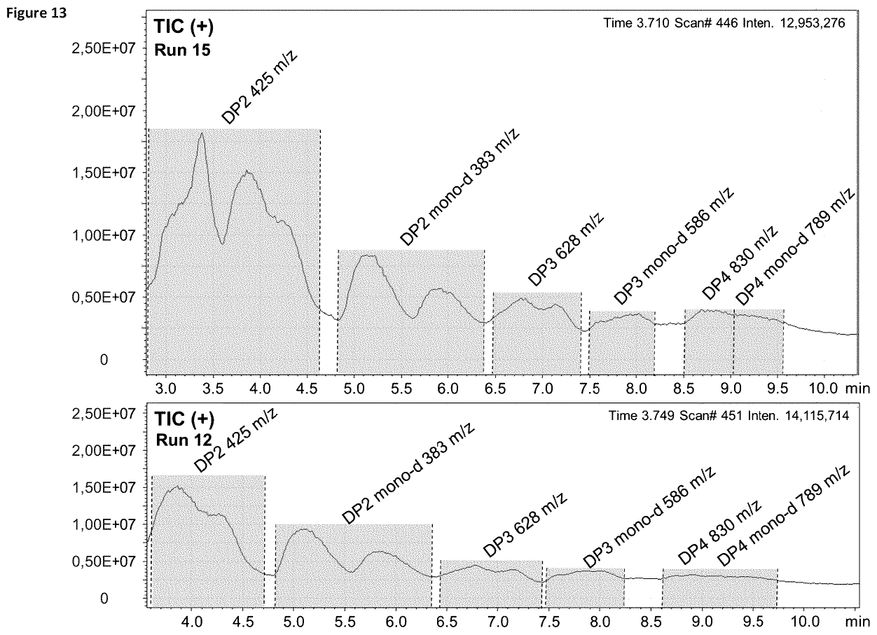

The reduced cubic model revealed further that factors A and C take part in multiple highly significant (p-value<0.0001) two factor (AB, AD, AE, BC, CD, CE) and three-factor interactions (ABE, BCD, BCE). Interestingly, no direct interactions between factor A and C were significant in the model. The overall highest conversion rate was achieved by run 19 (2 μM Factor A; 502 nmol/mL×h). Hydrolysis products were subjected to analysis by TLC (FIG. 12) and LC-MS (FIG. 13) in order to identify products and to reveal potential alterations in product properties with regards to the DP.

The relative distribution of DP2, DP2 mono-d, DP3, DP3 mono-d, DP4 and DP4 mono-d was anticipated as a further responses to evaluate the model in order to identify chitinase mixtures to predict the DP and to improve the production of chitin oligomers with a DP>2. Therefore the individual compound peaks of the LC-MS spectra were integrated for the design runs to use the relative abundances as responses. However, 16 runs could not be evaluated quantitatively as the overall product yield was too low to carry out a peak integration (runs 3, 10. 17, 20, 24, 25, 27, 34, 35, 36, 39, 43 and 45-48). The response for these runs was therefore set to zero. However, ANOVA evaluation resulted in non-statistically significant models.

The mean was calculated for 10 representative runs and confirmed that the overall proportional distribution of chitin oligomers is similar between the individual runs (Table 6.3). Therefore, it was concluded that

TABLE 6.3
Average proportional product composition of calculated from integrated
peak areas of 10 representative runs from the chitinase mixture design.

| | Average |
|---|---|
| Compound | proportion |
| DP2 | 37.2 ± 2.4 |
| DP2 mono-deacetylated | 20.0 ± 6.4 |
| DP3 | 6.4 ± 2.5 |
| DP3 mono-deacetylated | 5.1 ± 2.0 |
| DP4 | 1.0 ± 0.1 |
| DP4 mono-deacetylated | 5.0 ± 0.4 |

the individual chitinase mixtures did not change the overall product composition and only affected the overall conversion rate. The optimization function in Design Expert was used to model enzyme mixtures to maximize the overall conversion rate of chitin to oligomeric chitin mixtures. Three mixture solutions for maximized chitin (S1-83 Max) oligomer rates were tested and quantitative data was compared to the values predicted by the model. In addition, three mixture solutions for minimized chitin oligomer rates (S1-83 Min) were tested to validate the model's predictability.

All solutions for maximized conversion rates included a mixture of factor A (chitinase 1) and factor B (chitinase 2) with factor A being the predominant component (Table 6.5). Solution 3 also introduced factor C (chitinase 3) as a minor component. All solutions were validated experimentally at a 1 mL scale in three technical replicates. The achieved rates (Rate ach.) were in accordance with the predicted values (Rate pred.) and a substantial increase in conversion rates were achieved compared to the best run in the design (run 19; 502 nmol/ml×h) (Table 6.4). The optimized mixture were able to improve the rates by 80% (S1 Max), 73% (S2 Max), 53% (S3 Max) relative to run 19. The results for minimized solutions further confirmed that the model could be used to carry out reliable predictions of conversion rates.

TABLE 6.4
Optimized chitinase mixtures suggested by the design model. Multiple solutions were recommended
and three (1-3 Max) predicting the highest rate of reducing sugars (Rate pred.) were selected
for validation. In addition, three solution (1-3 Min) for minimal rates were included to validate
the predictive power of the model. Analysis was carried out in three technical replicates (n
= 3). A: chitinase 1, B: chitinase 2, C: chitinase 3, D: chitinase 4, E: chitinase 5.

| | Factor A | Factor B | Factor C | Factor D | Factor E | Rate pred. | Rate ach. |
|---|---|---|---|---|---|---|---|
| Solution | [μM] | [μM] | [μM] | [μM] | [μM] | [nmol/ml × h] | [nmol/ml × h] |
| 1 Max | 1.7 | 0.3 | 0 | 0 | 0 | 869 | 904 ± 27 |
| 2 Max | 1.6 | 0.2 | 0.1 | 0 | 0 | 843 | 869 ± 26 |
| 3 Max | 1.8 | 0.2 | 0 | 0 | 0 | 753 | 769 ± 15 |
| 1 Min | 0 | 0.4 | 0 | 0.6 | 1.1 | 3.9 | 20 ± 1 |
| 2 Min | 0 | 0 | 0 | 1.8 | 0.2 | 4.1 | 18 ± 1 |
| 3 Min | 0 | 0.6 | 0.7 | 0 | 0.7 | 8.6 | 19 ± 1 |

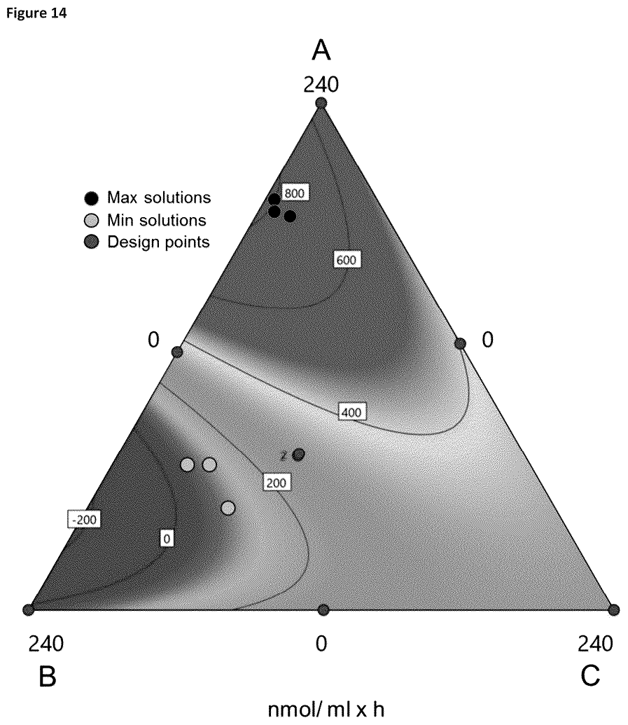

A response plot of the mixture model is displayed in FIG. 14. Tested optimized mixtures and corresponding responses for all solutions (1-3 Max black dots; 1-3 Min grey dots) were included in the plot indicating that the Max solutions were meeting the highest achievable conversion rates within the design constrains. Thus the response plot displays that the model could be used to successfully predict optimum enzyme combinations for maximized rates. Tested solutions for minimized rates were included to further validate the models predicative power and the response plot could be used to successfully predict enzyme combination to achieve minimum rates.

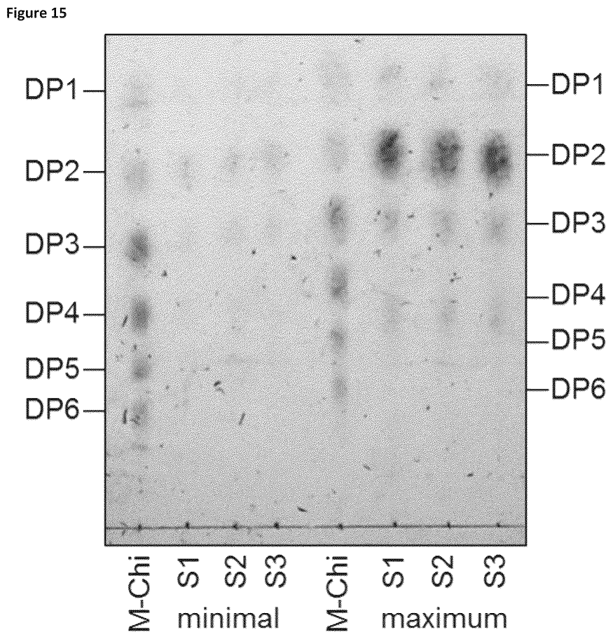

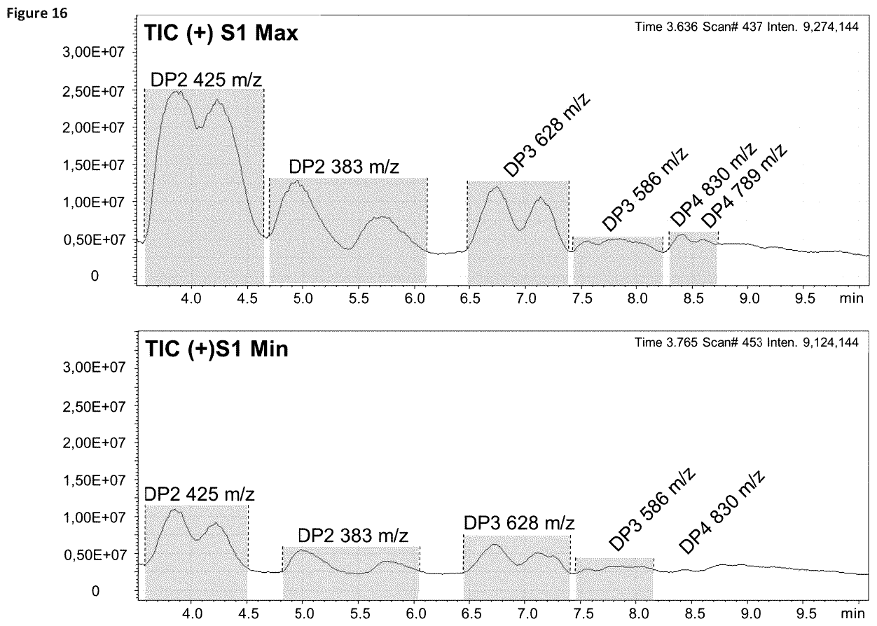

Product analysis by TLC and LC-MS further revealed the composition of the product mixture (FIG. 15 and FIG. 16). Product compositions of S1 Max-S3 Max were identical and did not change from the composition of the design runs. Chitin oligomers with DP2 were the predominant product and compounds with DP1, DP3 and DP4 were identified at lower quantities by TLC. LC-MS data further revealed the presence of partially deacetylated chitin oligomers that could not be detected by TLC due to the limited separative resolution and lack of partially deacetylate standard molecules.

Product analysis by TLC and LC-MS further revealed the composition of the product mixture (FIG. 15 and FIG. 16). Product compositions of S1 Max-S3 Max were identical and did not change from the composition of the design runs. Chitin oligomers with DP2 were the predominant product and compounds with DP1, DP3 and DP4 were identified at lower quantities by TLC. LC-MS data further revealed the presence of partially deacetylated chitin oligomers that could not be detected by TLC due to the limited separative resolution and lack of partially deacetylate standard molecules.

The overall substrate conversion yield was determined for all maximized solutions after determining the residue substrate after the reaction, relative to the starting material. With solutions S1 Max, the highest substrate conversion yield of 28.9±0.7% (28.9 mg/mL) and an improvement to 58% compared to run 19 (18.3±0.5%) was achieved.

In conclusion, the chitinase mixture model was used successfully to increase the overall conversion rates substantially relative to single enzyme reactions. However, the model could not be used to predict the DP of products and thereby altering product properties.

### Discussion

The design-of-experiments approach enables the assessment of complex biological systems using solely statistical modelling. Thereby, significant factors and factor interactions, optimum factor combinations and rate projections can be carried out for real systems. In order to obtain a robust process modeling, it is essential to carry out a factor pre-selection and to optimize the system focusing on the most crucial factors, thereby improving overall significance of the models. For this research, the different chitinolytic enzymes were analyzed to develop two individual mixture models to assess enzyme-substrate interactions and to tailor optimized enzyme cocktails for improved product rates. Furthermore, it was investigated whether the different enzyme combinations had a significant effect on altering product distributions in terms of degree of polymerization (DP). Evaluation of the model revealed that different combinations of chitinases had significant effect on the product rates. Significant factors and factor interactions were identified that indicated synergistic enzyme effects.

The chitinase mixture model revealed that all main factors (chitinase 1-5) as well as multiple two-factor and three-factor interactions had a significant impact on the overall product rate. The evaluation of the model by using the relative abundance of chitin oligomers with DP2, DP3 and DP4, that is with two, three or four GlcNAc units (i.e. as a response demonstrated that the product spectrum could not be changed by using different enzyme mixtures). This underlines that all implemented chitinases hydrolyze chitin by an overall related reaction mechanism. Therefore, further analysis of the model focused on the maximization of the production rate.

In order to assess the effect on the product rate more accurately, the optimization function in the DesignExpert software was used to specify enzyme cocktails for maximized conversion rates. Three solutions with the highest predicted rates were validated. Furthermore, the testing of minimized solutions was carried out to further validate the predictability of the model and achieved data was in agreement with the predicted rate. All tested mixtures revealed that chitinase 1 (factor A, SEQ ID NO: 1) had the strongest positive impact on the production rate and chitinase 2 and 3 (factor B, SEQ ID NO: 2; and factor C, SEQ ID NO: 3) enhanced the overall conversion. The predicted rates exceeded the highest yield achieved in the design (run 19) by 73% and experimental validation of all solutions were in great accordance with the predictions. An explanation for the substantial increase of activity by adding minor amounts of chitinase 2 to chitinase 1 is a synergistic effect coming from the different enzyme domains. Both enzymes contain different chitin-binding domains that are responsible for increased accessibility of the active site to the substrate. As chitinase 2 has an overall low chitinolytic activity, the chitin-binding domain could potentially enhance the overall accessibility for chitinase 1 resulting in an increased overall conversion rate.

So far, chitinolytic enzymes from different bacterial or fungal sources were tested for the degradation of chitin to oligomers yielding a broad spectrum of different oligomers. Typically, chitin is first pre-treated using harsh chemical or mechanical methods in order to break the crystal structure and to increase the overall substrate availability. In comparison to the data reported in the literature, the optimized enzyme cocktails were able to generate similar overall product yields ranging from DP1-DP4. Thus, even higher product rates and yields can be expected from the optimized enzyme cocktails using e.g. chemically pre-treated chitin such as colloidal chitin as it provides a higher surface area and lower crystallinity compared to chitin powder.

In summary, the implementation of the chitinase mixture model successfully revealed significant enzyme interdependencies and the model could predict optimized enzyme combinations that allowed a substantial increase in conversion rates of chitin powder compared to single enzyme reactions. The reliable predictive power even above the design constrains (502 nmol/ml×h) of the model further demonstrated that DoE can be used to develop highly specific enzyme cocktails that allow a substantial increase of conversion efficiency. The designs were furthermore carried out and evaluated within two working days leading to the optimized mixtures.

### Conclusions

The mixture models for chitinases were capable to identify significant main factors and factor interactions between the enzymes. Optimized enzyme mixture suggested by the design were validated and it was revealed that highly specific enzyme mixtures are required to achieve a substantial increase of the conversion rates. The design's predictability was exceptionally reliable as extrapolated responses with a rate increase of up to 58% were achieved. Such specific mixtures could not be determined strategically using the OFAT approach as it does not take any factor interactions into consideration. Furthermore, using the DoE approach all optimum mixtures were determined systematically using a minimum amount of individual experiments making it a time- and resource-efficient approach. Optimized mixtures were furthermore capable to produce mono-deacetylated COS of DP2-DP4 from chitin powder with overall yields of 28.9% which is competitive to current chemical degradation reactions.

### Experimental Section

### Materials

All chemicals used in the study were purchased from Carl-Roth (Germany) and were of highest purity; Chelating Sepharose FF was from GE Healthcare (Sweden). All buffers were prepared in demineralized water.

### Recombinant Production of Enzymes

Previously cloned constructs for chitinases (Example 2) were all transformed in E. coli BL21 (DE3) cells and expressed recombinant Starter cultures in 100 mL TB medium were cultivated overnight (180 rpm, 37° C.) and 10 mL of these culture were used to inoculate the main cultures (OD600 nm: 0.1). Ultra Yield™ flasks (2.5 L; Thomson Instrument Company, Oceanside, USA) were used for propagation of the main culture in 1000 ml TB-medium at 37° C. in orbital shakers and a shaking velocity of 200 rpm. Induction was carried out in the middle of the log-phase after 4 h of cultivation (OD600=4) by addition of 1 mM isopropyl-s-D-thio-galactopyranoside (IPTG) to the culture broth and temperature was then reduced to 28° C. Cells and supernatant were separated by centrifugation (8000×g, 30 min) after a total cultivation time of 18 h was reached.

Purification of Enzymes from Culture Supernatant

The protocol for the purification of 6×His tagged chitinases was adapted from Example 2 and two sequential steps were carried out:
Step 1. Ammonium sulfate precipitation
Solid ammonium sulfate was slowly added to 950 ml culture supernatant to a final concentration of 70% and was stirred for 2 h at room temperature. The precipitate was collected by centrifugation (8000×g, 30 min), dissolved in 100 ml PBS (pH 8.0) and filtrated (0.45 μm).
Step 2. Immobilized metal affinity chromatography (IMAC)
Dissolved samples from step 1 were applied to a Chelating Sepharose FF (GE Healthcare, Sweden) resin (column volume (CV) 15 ml) charged with 0.2 M NiSO4. The column was connected to an ÄKTA pure 25 (GE Healthcare, Uppsala, Sweden) and equilibrated using 3 CV PBS (pH 8.0). Samples were injected directly using a
sample pump and followed by a washing-step with equilibration buffer until the UV280 nm signal fell below 30 mAU. Weakly bound unspecific protein was eluted from the column using 50 mM imidazole in PBS (pH 8.0) followed by a second elution step at 250 mM imidazole in PBS (pH 8.0). All steps were carried out at a constant flow rate of 5 ml min-1. Elution fractions of 2 ml containing the respective enzyme were pooled and dialyzed against MAT buffer (33 mM TRIS, 33 mM CAPS, 33 mM MES [pH 8]). Proteins were quantified by the bicinchoninic acid assay (BCA) using bovine serum albumin as standard for calibration.

### Substrate Preparation

Chitin from shrimp shells (˜400.000 g/mol; Carl Roth) was mechanically pre-treated using a GyroGrinder (Fritsch GmbH, Germany) at 6000 rpm and thereby converted to a homogeneous powder of 80 μM average particle diameter. The powder was used without any further treatment as a substrate for all chitinolytic degradation experiments.

### Quantification of Chitinase and Chitin-Deacetylase Activity

Enzymatic activity of recombinant chitinases was determined by carrying out a reducing end assay [J., H., Svein, E., and H., V. G., Carbohydr Polym, 2004. 56(1): p. 35-39.]. Hydrolysis samples were centrifuged at 8000×g for 2 min and 40 μl of the supernatant was mixed with 40 μl 0.5 M NaOH and 40 μL of a aqueous MBTH reagent solution (1.5 mg/ml 3-Methyl-2-benzothiazolinonhydrazon and 0.75 mg/ml Dithiothreitol). Samples were incubated at 80° C. for 15 min and then mixed with 80 μl developing solutions (0.5% (w/v) FeNH4(SO4)2)×12H20, 0.5% (w/v) sulfamic acid and 0.25 M HCl). Samples were cooled down to room temperature and absorption was determined at 620 nm. A calibration curve of N-acetyl-glucosamine was prepared freshly for each measuring cycle. One activity unit [U] was defined as the amount of enzyme required to release 1 nmol of reducing sugars per hour.

A commercial acetic acid assay kit (K-ACETRM; Megazyme, Bray, Ireland) was used in multitier plate format measuring the amount of released acetate during CDA reactions. A calibration curve was generated freshly for each measuring cycle using acetic acid as a standard. One activity unit [U] was defined as the amount of enzyme required to release 1 μg of acetic acid per hour.

### Product Analysis by Thin-Layer-Chromatography and LC-MS

For the identification of hydrolysis products, soluble samples were subjected to TLC analysis using 10×10 cm TLC Silica gel 60 F254 plates (Merck, Darmstadt, Germany) and a mixture of butanol:methanol:25% ammonia:H2O (5:4:2:1) as mobile phase. A total volume of 0.3 μl for samples and chitin standard sugars (Megazyme, Chicago, USA) (1 mg/ml) were applied to the plate. After separation, the plate was air-dried and developed using a solution containing 200 ml Acetone, 30 ml phosphoric acid (85%), 4 ml aniline and 4 g diphenylamine. Spots were made visible using a heat gun at 300° C.

The protocol for analysis by LC-MS was developed based on the method described by Hamer et al. [Sci Rep, 2015. 5: p. 8716.]. Analysis of soluble oligomeric samples was carried out using a Shimadzu LC-30 AD system coupled to a SIL-30AC autosampler, a CTO-20AC column oven and a LCMS-2020 mass spectrometer (Shimdazu, Kyöto, Japan). A sample volume of 1 μl was separated by hydrophilic interaction chromatography using an Acquity UPLC BEH Amide column (1.7 μm, 2.1×150 mm) coupled to an Acquity UPLC BEH Amide 1.7 μm VanGuard pre-column (2.1×5 mm) (both Waters Corporation, Milford, USA). Flowrate was set to constant 0.5 ml min-1 and the column oven temperature to 30° C. Samples were eluted from the column with a gradient of A (Acetonitrile+0.1% (v/v) formic acid) and B (water). Sample separation was done over 16 min using the following gradient: 0-2.5 min isocratic 80% A; 2.5-12.5 min, linear from 80% to 35% (v/v) A, followed by column re-equilibration 12.5-13.5 min, linear from 35%-80% A (v/v); 13.5-16.0 min, isocratic 80% A. MS-detection was carried out in positive mode with the interface voltage at 4.5 kV, the nebulizer gas flow rate at 1.5 L/min, drying gas flow at 15.0 L/min and the dry temperature at 249° C. Mass spectra were recorded over a scan range from m/z 100-1500.

Peaks from target compounds were identified and integrated automatically using the Chromatopac algorithm of the post run analysis software (Shimadzu). Peak smoothing was inactivated and the baseline following degree was set to 1 while disabling the baseline correction method. Noise was calculated using the ASTM method.

### Design-of-Experiment Models

A mixture design with cubic model order was generated including all five chitinases as variable factors using the Design Expert 11 software (Stat-Ease inc. Minneapolis, USA) in order to optimize enzyme combinations for improved conversion efficiency of chitin powder to COS. In addition, the design was used to assess whether product properties can be altered in order to yield single chitin oligomers. A total molar concentration range of 0-2.0 μM (representing a volume of 0-240 μl) was selected for all chitinases (Table 6.8). The total reaction volume

TABLE 6.8
Factors and range limits used in the chitinase mixture design. The Upper
limit (240 μl) represents the maximum enzyme concentration of 2 μM.

| Factor | Name | Lower limit [μl] | Upper limit [μl] |
|---|---|---|---|
| A | Chitinase 1 | 0 | 240 |
| B | Chitinase 2 | 0 | 240 |
| C | Chitinase 3 | 0 | 240 |
| D | Chitinase 4 | 0 | 240 |
| E | Chitinase 5 | 0 | 240 |

was fixed to 1000 μL for all experiments and within this volume the total molar enzyme concentration was set. The special cubic model was created in order to assess three-factor interactions resulting in a total of 48 runs. All runs were prepared in 2 mi centrifuge tubes using 100 mg of ground chitin powder at a working volume of 1000 μl using MAT buffer at pH 8. Samples were incubated at 30° C. for 16 h in a thermomixer at 1000 rpm. After incubation, a reducing end assay was carried out to determine the total amount of reducing sugars and TLC was used to assess changes in product properties. These responses were used to evaluate the design by analysis of variance (ANOVA) within the Design Expert 11 software revealing optimum enzyme combinations.

### Optimized Depolymerization and Deacetytation Reactions

Optimized chitinase and CDA mixtures were used to carry out sequential depolymerization and deacetylation reactions. Therefore, chitin powder (100 mg chitin) was incubated for 16 h to carry out the chitinolytic enzyme reaction first using one optimized chitinase mixture at a total molar concentration of 2 μM at pH 8 and 30° C. Subsequently, the chitinolytic reaction was stopped by heat inactivation of enzymes (95° C. 10 min). The second reaction was started by addition of 2 μM of the optimized deacetylase mixture and again incubated for 16 h at 30° C. Enzymes were again deactivated as described above and products were analyzed after freeze-drying using TLC and LC-MS.

### Quantification of Products

Individual oligomeric chitin and chitosan products were quantified using TLC by establishing calibration curves of monomeric, dimeric, trimeric and tetrameric chitin standard oligosaccharides within a concentration range of 0.3125, 0.625, 1.25, 2.5 and 5 mg/ml. A total standard volume of 1 μl was applied and analysis was carried out as described. A densitometrical sample quantification was carried out using the AIDA image analyzer software (Raytest Isotopenmessgeräte GmbH, Straubenhardt, Germany).

### Conclusions on Examples 1-3

Next generation sequencing of the P. orarium genome was carried out and the assembled data was used for gene mining of homologous and characteristic enzyme domains for chitinases (glycosyl hydrolases) and chitin-deacetylases (NodB). In Example 2, five different target genes for chitinases were cloned successfully into E. coli BL21 as different constructs using the golden gate cloning technology. Constructs comprising a PeIB signal peptide for secretion and 6×His tag for purification by IMAC were identified as the most suitable candidates for expression. Purified chitinases were characterized using chitin powder regarding their pH, temperature and salt optima and their respective substrate specificity and kinetics. Molecular masses and chain lengths of oligomeric products were determined. It was determined that the novel enzymes differ in size from typical reported chitinases and exhibit in general higher pH (8-10) and lower temperature (30-40° C.) optima. Furthermore, salt contents of 1-5% (w/v) NaCl were beneficial for enzyme activity. Tetrameric trimeric, dimeric and monomeric oligomers were mostly obtained after enzymatic digestion of chitin and colloidal chitin in different quantity distributions. The results suggested that the novel enzymes can be used for a biological degradation process at moderate temperatures of insoluble chitin to chitin-oligomers, although with a rather limited range of DP.

The chitinases and chitin-deacetylases were used to explore a fully enzymatic conversion process to directly generate partially deacetylated COS from chitin powder. Thus, specific enzyme cocktails were developed using the design-of-experiments approach to elucidate optimum enzyme combinations for maximized production rates and putative changes on product characteristics with regards to DP and DA. In Example 3, mixture designs for chitinases were used to generate enzyme mixtures to maximize conversion rates. It was discovered that total conversion rates could be increased by 80% for the chitinase mixture compared to single and non-optimized enzyme reactions. Different enzyme mixtures did however not alter the overall product composition concerning DP.

Compared to current chemical COS production processes yields of the novel enzymatic process are in a similar range and thus the process can be considered efficient. Furthermore, the DoE approach demonstrated to be a more useful and rapid tool than the OFAT approach for modelling complex multi-enzyme reactions and to determine optimum enzyme combinations to maximize conversion rates.

### Example 4

The protective activity of chitinolytic enzymes against fungi infestation was tested. To this end, the chitinolytic enzymes identified and characterized in Example 2 were produced in E. coli BL21 cells. The enzymes were purified from the culture supernatant.

The start concentration of each chitinase used in minimum inhibitory concentration (MIC) tests was 100 μg/ml.

The direct effect in the MIC test on different fungal pathogens was assessed. All chitinases tested were able to inhibit the growth of fungal pathogens:

| | | | Fusarium |
|---|---|---|---|
| Sample | Septoria tritici | Gibberella zeae | oxysporum |
| Chitinase 1 | xx | x | xx |
| Chitinase 2 | xx | xx | xx |
| Chitinase 3 | nd | x | xx |
| Chitinase 4 | nd | x | xx |
| Chitinase 5 | xx | xx | xx |

xx = no growth/strong inhibition;
x = small inhibition;
nd = not determined

Chitinase 1 was also tested in the MIC assay against Fusarium culmorum and showed inhibition as well. The chitinases were further tested for their protective activity in germination tests. Briefly, the following steps were conducted:
1. Disinfection of wheat seeds in 10% bleach for 10 minutes in a biosafety cabinet
2. Germination on filter paper
3. Foliar application 3 dps (days post seeding) and 1 dbi (day before infection)
4. 4 dps: add pathogen Fusarium culmorum
5. Monitoring germination and plant health/phytotox

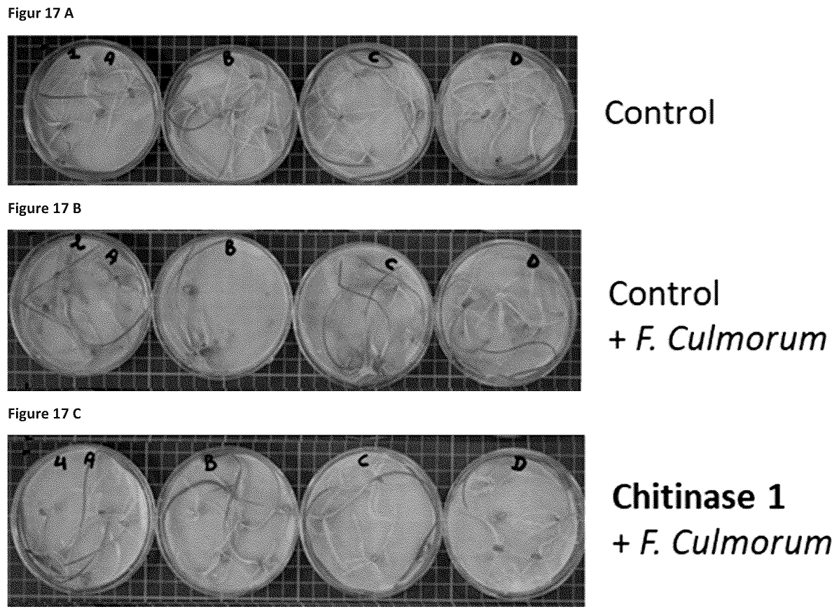

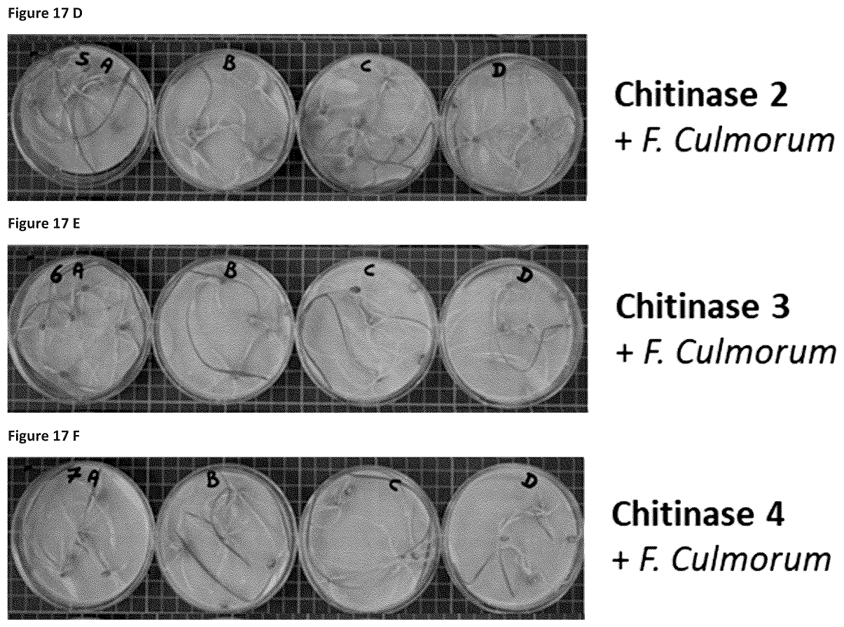

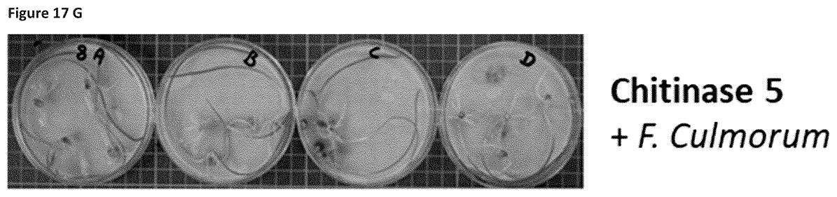

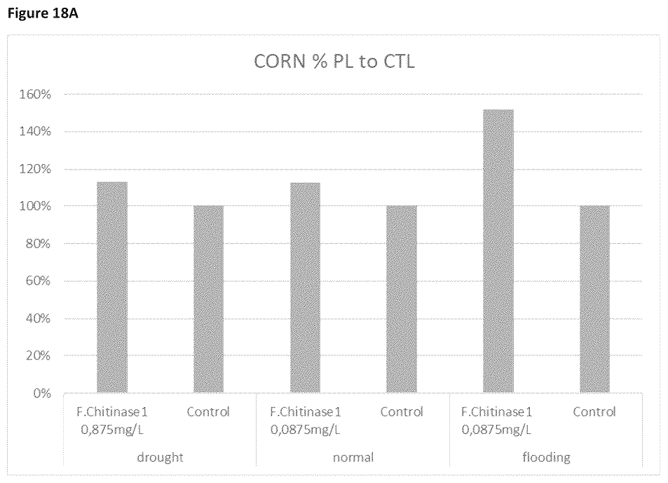

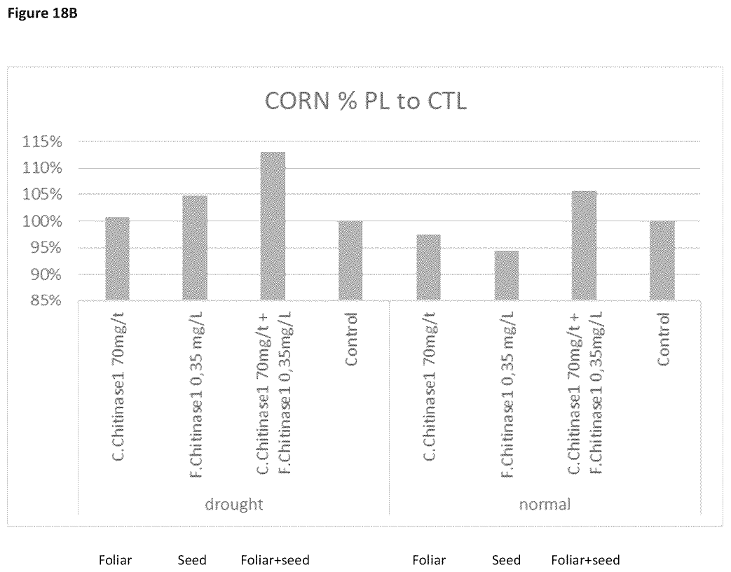

Results are shown in FIGS. 17A-17G. The results show that addition of Chitinase 1 resulted in strong inhibition of fungal growth and improved plant health (a biostimulant effect compared to the control).

These results thus show that chitinases, and in particular the chitinases described herein, can efficiently inhibit growth of pests on plants, such as fungi. Plant health can thereby be improved. Thus, chitinases can be used as plant protection agents as described herein.

### Example 5

The effects of chitinases on insects was tested on D. melanogaster eggs. Briefly, eggs were treated with chitinase 1 or a control, and larvae survival was assessed 3 days after treatment (DAT). Results for chitinase concentrations of concentration from 10% to 0.1% were as follows:

| Sample | % survival | % mortality |
|---|---|---|
| Chitinase 1 | 64 | 36 |
| Control | 75 | 25 |

These results thus show that chitinases, and in particular the chitinases described herein, can decrease the survival of pests, such as insects. This further supports that chitinases can be used as plant protection agents as described herein.

### Example 6

Chitinase 1 was tested for protective effects against abiotic stress. Abiotic stresses tested were drought, flooding, salt, or frost stress. Plants tested were: corn (maize), rice, barley, pear and apple. Test conditions were as follows:

### Drought Stress

plants are drilled in trays
germination at 25° C./15° C. (day/night) light 16 h, dark 8 h
application 10 days later
2 days later replanted in pots of 8×8 cm
3 possible regimes
   no water
   every day water with flooding system
   continuous water

### Salt Stress

plants are drilled in trays
germination at 25° C./15° C. (day/night) light 16 h, dark 8 h
application 10 days later
2 days later replanted in pots of 8×8 cm
3 possible regimes
   direct salt stress on moment of replanting
   salt stress 4 days after replanting
   salt stress 7 days after replanting
salt stress=100 mL of a solution of 120 g/L NaCl

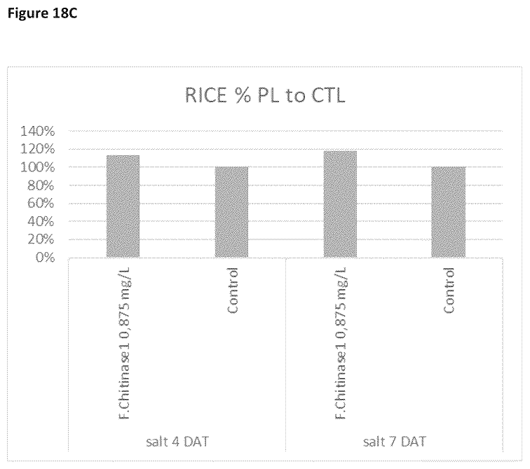

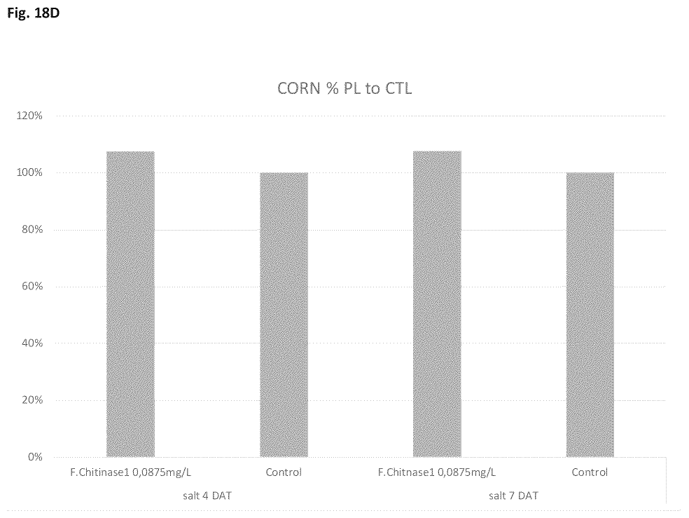

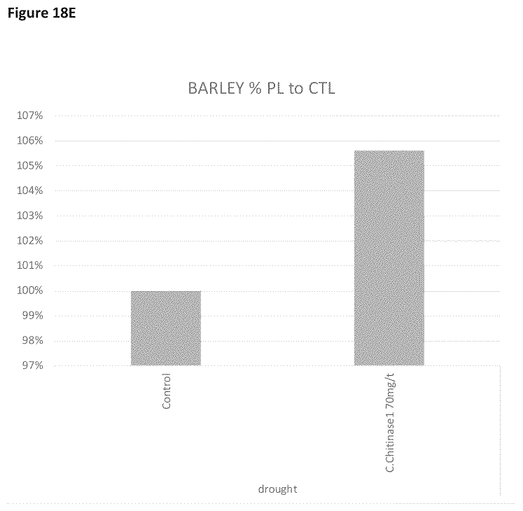

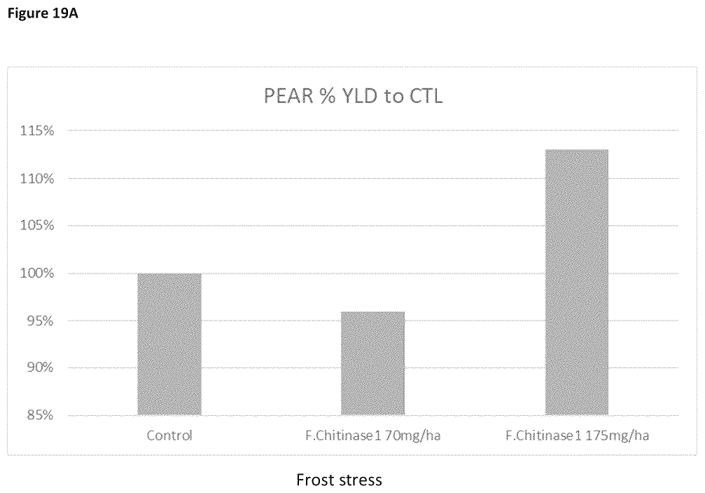

Results in terms of plant length (PL) are shown in FIG. 18 after foliar application, seed application or combined foliar and seed application. Normalized to control (CTL).

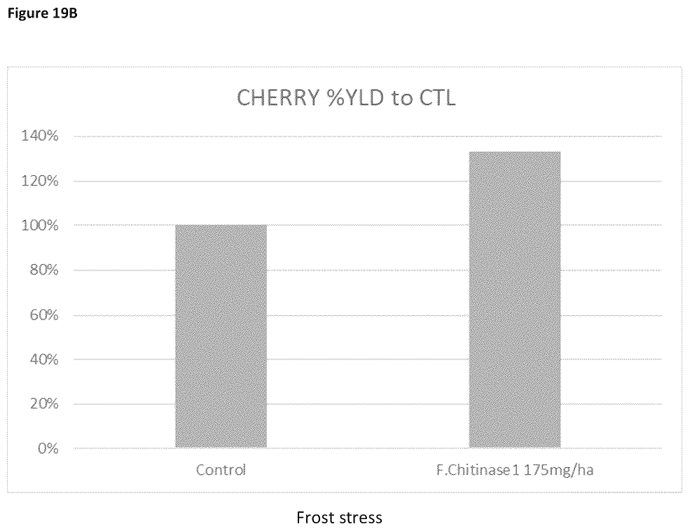

Moreover, FIG. 19 shows results on pear and cherry yield after foliar application of chitinase 1 upon frost stress.

The results clearly show that application of chitinase 1 to different plants protects the plants from different abiotic stresses, including drought, flooding, salt and frost. Without wishing to be bound a particular theory, one explanation for this surprising finding is that chitinases, and in particular chitinase 1, can activate plant defense mechanisms against abiotic stresses.

### INDUSTRIAL APPLICABILITY

The chitinolytic enzymes described herein, and in particular for a use thereof in plant protection, as well as the related products and uses described herein can be applied, for example, to commercial plant protection agents e.g. for use in agriculture. The present disclosure is thus industrially applicable.

1. A chitinolytic enzyme comprising a first amino acid sequence that is at least 70%, such as 100%, identical to an amino acid sequence selected from the group of SEQ ID NOs: 1-5.

2. The chitinolytic enzyme according to claim 1, wherein the chitinolytic enzyme further comprises a second amino acid sequence fused to the N-terminus of the first amino acid sequence.

3. The chitinolytic enzyme according to claim 2, wherein the second amino acid sequence is less than 50 amino acids in length.

4. The chitinolytic enzyme according to claim 2 or 3, wherein the second amino acid sequence consists of a signal peptide, preferably a PelB leader sequence (SEQ ID NO: 6).

5. The chitinolytic enzyme according to any one of claims 1-4, wherein the chitinolytic enzyme further comprises a third amino acid sequence fused to the C-terminus of the first amino acid sequence.

6. The chitinolytic enzyme according to claim 5, wherein the third amino acid sequence is less than 50 amino acids in length.

7. The chitinolytic enzyme according to claim 5 or 6, wherein the third amino acid sequence consists of a purification tag, preferably a 6×His tag (SEQ ID NO: 7).

8. The chitinolytic enzyme according to any one of claim 2-7, wherein the enzyme comprises or consists of a first, a second and a third amino acid sequence.

9. The chitinolytic enzyme according to any one of claims 1-8, comprising the amino acid sequence according to SEQ ID NO: 1.

10. The chitinolytic enzyme according to any one of claims 1-8, comprising the amino acid sequence according to SEQ ID NO: 2.

11. The chitinolytic enzyme according to any one of claims 1-8, comprising the amino acid sequence according to SEQ ID NO: 3.

12. The chitinolytic enzyme according to any one of claims 1-8, comprising the amino acid sequence according to SEQ ID NO: 4.

13. The chitinolytic enzyme according to any one of claims 1-8, comprising the amino acid sequence according to SEQ ID NO: 5.

14. The chitinolytic enzyme according to any one of claims 1-13, essentially consisting of a first amino acid sequence that is at least 70%, such as 100%, identical to an amino acid sequence selected from the group of SEQ ID NOs: 1-5.

15. The chitinolytic enzyme according to any one of claim 1 or 9-14, consisting of a first amino acid sequence that is at least 70%, such as 100%, identical to an amino acid sequence selected from the group of SEQ ID NOs: 1-5.

16. A nucleic acid encoding the chitinolytic enzyme according to any one of claims 1-15.

17. A vector comprising the nucleic acid according to claim 16.

18. The vector according to claim 17, which is an expression vector.

19. A host cell comprising the nucleic acid according to claim 16 or the vector according to claim 17 or 18.

20. The host cell according to claim 19, which is a plant cell or a microbial cell.

21. The host cell according to claim 20, wherein the plant is an arable crop, fruit-bearing plant or vegetable.

22. The host cell according to claim 20, wherein the microbial cell is a bacterial cell.

23. The host cell according to claim 22, wherein the bacterium is E. coli.

24. A plant comprising the chitinolytic enzyme according to any one of claims 1-15, or the nucleic acid according to claim 16, or the vector according to claim 17 or 18.

25. A method for producing a chitinolytic enzyme according to any one of claims 1-15, the method comprising culturing a host cell according to any one of claims 19-23.

26. The method according to claim 25, further comprising harvesting the cells and/or the supernatant during and/or after the culture, preferably harvesting the supernatant after the culture.

27. The method according to claim 25 or 26, further comprising purifying the chitinolytic enzyme.

28. A composition comprising at least one chitinolytic enzyme according to any one of claims 1-15.

29. The composition according to claim 28, wherein the composition comprises at least two different chitinolytic enzymes according to any one of claims 1-15.

30. The composition according to claim 29, wherein the at least two different chitinolytic enzymes exert a synergistic effect.

31. The composition according to claim 30, wherein the synergistic effect is characterized by a disproportionally improved chitin degradation rate (compared to the individual enzymes).

32. The composition according to any one of claims 28-31, comprising a chitinolytic enzyme comprising or (essentially) consisting of SEQ ID NO: 1 and a chitinolytic enzyme comprising or (essentially) consisting of SEQ ID NO: 2.

33. The composition according to any one of claim 32, further comprising a chitinolytic enzyme comprising or (essentially) consisting of SEQ ID NO: 3.

34. The composition according to any one of claims 28-33 that is a plant protection agent.

35. Use of a composition comprising at least one chitinolytic enzyme as a plant protection agent.

36. Use of the composition according claim 35, wherein the composition is according to any one of claims 28-35.

37. Use of the composition according to claim 35 or 36 as a plant protection agent against an organism that contains chitin.

38. Use of the composition according to any one of claims 35-37, wherein the plant protection agent is against a fungus and/or against an insect.

39. Use of a composition according to any one of claims 35-38, wherein the fungus is a Fusiarum or Septoria species.

40. Use of the composition according to claim 35 or 36 as a plant protection agent against abiotic stress, wherein the abiotic stress preferably is drought, frost or flooding stress.

41. Use of the composition according to claim 35 or 36 as a biostimulant against abiotic stress in plants, wherein the abiotic stress preferably is drought, frost or flooding stress.

42. Use according to any one of claims 35-41, wherein the plant is an arable crop, fruit-bearing plant or vegetable.

43. A method of protecting a plant from pests and/or abiotic stress, the method comprising the application of a composition comprising at least one chitinolytic enzyme on the plant or a part thereof.

44. The method of protecting a plant from pests according to claim 43, wherein the composition is according to any one of claims 28-35.

45. The method according to any one of claim 43 or 44, wherein the plant or plant part is soaked in the composition.

46. The method according to any one of claims 43-45, wherein the composition is applied by spraying to a surface of the plant or part of the plant, such as a leave or seed.

47. The method according to any one of claims 43-46, wherein the pest is an organism that contains chitin.

48. The method according to any one of claims 43-47, wherein the pest is a fungus or an insect.

49. The method according to claim 48, wherein the fungus is a Fusiarum or Septoria species.

50. The method according to any one of claims 43-49, wherein the plant is an arable crop, fruit-bearing plant or vegetable.

51. The method according to any one of claims 43-50, wherein the abiotic stress is drought, frost or flooding stress.


SEQUENCE LISTING (19 sequences)

SEQ ID NO: 1 - protein, 815 aa, Photobacterium orarium
  Chitinase 1
MAAPGTPQIAWMETDYAIVEVDQAASAYKSLITVKPAAEVPVAWQRYSGETADLWKVKLNGNVVFEQSIAPASSGAGSTTLSVAQGGQYAMTVELCSGTGAAQACTSSAATNIVVADTDGSHLDPLPMNVDANNGNYTTPANTVVGAYFVEWGVYGRKFSVDNIPAQNLTHILYGFIPICGPNESLGEIENGNSLAALNRACQGTPDYEVVIHDPWAAVQMPQPQSGHTHSTPYKGTYGQMMALKQRYPDLKIVPSVGGWTLSDPFYSFTDKAKRDIFVASVKKFLKTWKFFDGVDIDWEFPGGKGANANLGDPVNDGPAYVALMRELRVMLDELEAETGRQYELTSAIGVGYDKIDVVNYADAVQYMDYIFAMTYDFFGGWNNVPGHQTALFCGSHMSADVCNGTGVDENGVPRQGPAYTAAHGIDRLLAQGVPANKLVLGTAMYARGWTGVTEASMTDPTNPMTGTGNGMVPGSWEPGVIDYKDVVTDYINNAAVTKGYDAQAEAPWAYNPSNGNLITYDDRRSVLAKGQYVRNLGLAGLFAWEIDADNGDILNAMQDSLAGGGGNYAPVAKAGADQVVNGAASVTLDGSASSDRDGQIVSYQWTQTSGPALTLTGSDTATATVAVPEVTADTQYAFTLTVTDNAGDTATDSVTVTAKAPGSNTAPVVAVSGPATASAGDTVVLDASQSSDADGDTLTFTWTVPAGVNATVNGATVSFVADSYTVDTPLNFTVAVSDGTDSVTESVTVTVLKDGGSTGCTNAWATGGVYNSGDIVTHSGKSWKAKWWTTGEEPGTTGEWGVWEDLGAANCQIE

SEQ ID NO: 2 - protein, 515 aa, Photobacterium orarium
  Chitinase 2
MATANYNPDGVYSAGEQVIYQGATYQALRSTENEAPDTYLGDAWQQVNAQNVTSSTTPTYPAYQSSAVYVGGDHVSYNSQIYKAKWWTQGEAPDATPGTGVWEWVSADNNPDPGPGPDPDPTPEPTPANGIIGQNPDGSYIMSKTYLDAREAELTSSPEFAAVLASISTRDNAVVEAVVPGLSTNPDNVKRVEALISEQKWDALFPERNVVYTYSNFLKAVAKFKGFCATYTDERAAQSDAICAKSLAVMFAHFTQETGAHNPHSPYEEWRQGLFFVREAGCSEEASSCGYNSECAASNWQTEQWPCGTNPDGSYVKYFGRGAKQLSYHYNYGPFSDFIFNDVNVLLQDPDRVADSWLNLASAVFFFVYPQPPKPSMLHVIDGTWQPTAADIAEKRVPGFGVTTMIINGGIECTLDTEKPQSVNRIKYYRGHAAALGVAIPADEQLGCAGMKAFKKTGDSTFGLYWENDWSYYPDNPGGSSFACRIESGYQTAHTTLKKGDYAKCIQKYYGVTIE

SEQ ID NO: 3 - protein, 826 aa, Photobacterium orarium
  Chitinase 3
MALAAPGTPQLAWMETDYAIVEVDQAATAYKDLVTVKNAADVPVAWQRYSGETADHWKVKLNGNVAFEESINPASSGAGSTTLSITQGGQYALTVELCSGSGAEEACSSSPATNIVVADTDGSHLDPLPMNVDPNNGTYSTPADTVVGAYFVEWGVFGRKFTVDKIPAQNLTHILYGFIPVCGPNESLGEIESGNSLAALNRACAGTPDFEVAIHDPWAAVQMPQPQSGHTYSTPYKGNYGQLMALKQRYPDLKVIPSIGGWTLSDPFFSFTDKSKRDVFVASVKKFLQTWKFFDGVDIDWEFPGGKGANTTLGDPVNDGPAYVALMRELRTMLDGLEAETGRQFELTSAIGVGYDKIDVVNYAEASQYMDYIFNMSYDFYGGWSNVTGHQTALNCGSHLTADQCNGTGVDENGEPRQGPAYTTAHGVERLLAQGVPANKLVVGAAMYGRGWTGVTQASMTDPSNPMTGVGNGAVAGSWEAGVIDYKDVVTRYENKAGVVLGYDEQAEAPWAYDPSNGDLVTYDSPRSIMAKGQYVRDLGLAGLFAWEIDADNGDILNAMQESLAGAPAGNRAPVARAGSDQQVNTAATVTLNGSSSTDSDGQIAGYQWVQTSGPALTLNGANTASATISVPDVTVDTQYVFTLTVTDNEGATATDSITVTAKAPGAQNTAPVASLTGPGTANADDVITLDASQSTDADGDTLTYDWTVPAGVNAVINGSSLSFTADSYTTDTALSFSVSVSDGTASDAASLTVTIAKDESGTGGEGDFPAYVEGTAYQAGDQVSNGGVNFECKPYPYSGWCSGAAWAYEPGVGVYWQDAWTQLIE

SEQ ID NO: 4 - protein, 777 aa, Photobacterium orarium
  Chitinase 4
MAQAAANCRPDGLYQTPGVTVPYCTVYDQDGREKMGVDHPRRVIGYFTSWRSGNDPQSSYLVNDIPWDSLTHINYAFVSIGSDGKVNIGNVNNPDNPAVGKEWPGVEIDPALGFKGHFGALATAKAQHGVKTLISIGGWAETGGHFDDNGNRVADGGFYTMTTNADGSINHQGIQTFADSAVAMMRQYKFDGLDIDYEYPTSMQGAGNPDDFAYSDAMRPHLMKSYHELMKVLREKLDQASAQDGHHYMLTIAAPSSGYLLRGMETMSVTKYLDYVNIMSYDLHGAWNDYVGHNASLFDSGLDAELEAGNVYGTAQYKKTGYLNTDWAYHYFRGSMPAGRINIGVPYYTRGWQGVTGGTNGLWGKAALPNQSDCPTGTGAGTSNCGYGAIGIDNMWHDKDANGNEMGAGSNPMWHAMNLANGVYGSYTAAYGLDPVNDPQDALTGTYTRHYDSVSVAPWLWNADKKVFLSTEDKESINTKADYVIEQGIGGIMFWELAGDYSCYNLDANGNRTTVDPTENACKTGNGEYHMGDTMTKAIHNKFATATPYGNTLAEGAIPTEAVNIDVSVTGFKVGDQNYPLNPTMTLTNKTGQTLPGGTEFQFDIPTSTPDNMSDQSGANLQVISSGHTRGDNIGGLDGNFHRVAFTLPSWKQLGNNESFELTLNYFLPISGPANYSVRVNNADYALAFEQPDLPIADLSNGGGDNGGGDGNNGDCVTTGVNTYPNWPQTDWAGNPSHAGTGDKIIYNGAVYQAKWWTNSVPGSDGSWDTVCTVQIE

SEQ ID NO: 5 - protein, 594 aa, Photobacterium orarium
  Chitinase 5
MAMGWSQININGVQKWEEAYSLNVDAANEIITIGAADTAGALYASQSLLQLVDGNKVPEVQITDAPRFAYRGFSVDAVRNFRTKDAIIQLLDQMAAFKLNKLHLRLADDEGWRIEIAGLPELTDVGATRCHDPEEKACILPFLGAGPDGSPESNGYYTATDYQDILSHAAALNIEVVPEIDIPGHAHAAIKAMEARYDHYAAQGNMAEANKYLLTDFNDTTQYLSVQMFTDNAINVCMESSYNFVDAVVSGLVSLHQGVQPLKTFHFGGDEIAGAWINSPACQDFIANNTDGVHSVSDLSRYFVERISVITANYGLDMAGWEDGLMHDGQVYPRSQMANNQLWGNAWQNIWEWGVADRAYNLANNDYKVVYNHATHLYFDHPYEPDPNERGYYWAPRFTDTRKTFGFMPDDVFANADFTRAGAPITKAEVVASAGVKKLLKPENVLGLQGSLWAEAVRTEDQFEGMIFPRVLGLAERAWHTATWEANDNAGIALDETGRNADYNHFANLLGQKVLPKLEQAGIAFNLPVPGGVIENGVLQANSTFPGLTIEYSTDQGTSWQSYDHLNPPAVAAGVQLRTVSGQRTSRVTTVNIE

SEQ ID NO: 6 - protein, 20 aa, Artificial Sequence
  PelB
MKYLLPTAAAGLLLLAAQPA

SEQ ID NO: 7 - protein, 6 aa, Artificial Sequence
  6xHis tag
HHHHHH

SEQ ID NO: 8 - DNA, 2445 bp, Photobacterium orarium
  Chitinase 1
atggccgcacctggtacaccgcagattgcatggatggaaaccgattatgcaattgttgaagttgatcaggcagcaagcgcatataaaagcctgattaccgttaaaccggcagccgaagttccggttgcatggcagcgttatagcggtgaaaccgcagatctgtggaaagttaaactgaatggtaacgtggttttcgagcagagcattgcaccggcaagcagcggtgcaggtagcaccacactgagcgttgcacaaggtggtcagtatgcaatgaccgttgaactgtgtagcggtacaggtgcagcacaggcatgtaccagcagcgcagcaaccaatattgttgttgcagataccgatggtagccatctggatccgctgccgatgaatgttgatgcaaataatggtaattataccacgcctgccaataccgttgttggtgcatattttgttgaatggggtgtgtatggtcgtaaatttagcgttgataatatccctgcacagaatctgacccatattctgtatggttttattccgatttgcggtccgaatgaaagcctgggtgaaattgaaaatggtaatagcctggcagcactgaatcgtgcatgtcagggtacaccggattatgaagttgttattcatgatccgtgggcagcagttcagatgccgcagccgcagagcggtcatacccatagcaccccgtataaaggcacctatggtcagatgatggcactgaaacagcgttatccggatctgaaaattgttccgagcgttggtggttggaccctgagcgatccgttttatagctttaccgataaagccaaacgcgatatctttgttgcaagcgtgaaaaagtttctgaaaacctggaaatttttcgacggcgtggatatcgattgggaatttcctggtggtaaaggtgcaaatgcaaatctgggtgatccggttaatgatggtccggcatatgttgcactgatgcgtgaactgcgtgttatgctggatgaactggaagcagaaaccggtcgtcagtatgaactgacaagcgcaattggtgttggctatgacaaaattgatgtggtgaattatgcagacgccgtgcagtatatggattatatctttgccatgacctatgattttttcggtggctggaataatgttccgggtcatcagaccgcactgttttgcggtagccatatgagcgcagatgtttgtaatggcaccggtgttgatgaaaatggcgttccgcgtcagggtcctgcatataccgcagcacatggtattgatcgtctgctggcacagggtgttccggcaaataaactggttctgggcaccgcaatgtatgcacgcggttggaccggtgttaccgaagcaagcatgaccgatccgaccaatccgatgaccggcaccggtaatggtatggttcctggtagctgggaaccgggtgttattgattataaagatgtcgtgaccgactatatcaataatgcagcagttaccaaaggttatgatgcacaggccgaagcaccgtgggcatataatccgagcaatggcaatctgattacctatgatgatcgtcgtagcgttctggcaaaaggccagtatgtgcgtaatctgggtttagcaggtctgtttgcatgggaaattgatgccgataatggcgatattctgaatgcaatgcaggatagtctggcaggcggtggcggtaattatgcaccggtggcaaaagccggtgcagatcaggttgttaatggtgcagccagcgttaccctggatggtagcgcgagcagcgatcgtgatggccagattgttagctatcagtggacccagaccagcggtccggcactgaccctgaccggtagcgataccgcaaccgcgaccgttgcagttccggaagttaccgcagatacccagtatgcatttacactgaccgttacagataatgccggtgatacagcaaccgatagcgttaccgttaccgccaaagcaccgggtagtaataccgcaccggttgttgccgtttcaggtccggcaaccgcaagcgcaggcgataccgtggttctggatgcaagccagagcagtgatgcagatggcgataccctgacctttacctggaccgttccggcaggcgttaatgcaaccgtgaatggtgcaaccgttagctttgttgccgatagctataccgtggatacaccgctgaattttaccgttgccgttagtgatggtacagatagcgtgaccgaaagtgtgaccgtgaccgttctgaaagatggtggtagtaccggttgtaccaatgcatgggcaaccggtggtgtgtataatagtggtgatattgtgacccatagcggcaaaagctggaaagcaaaatggtggaccaccggtgaagaaccgggtacgacaggtgaatggggagtttgggaagatctgggtgcagcaaattgtcagatcgaa

SEQ ID NO: 9 - DNA, 1545 bp, Photobacterium orarium
  Chitinase 2
atggccaccgcaaactataatccggatggtgtttatagtgccggtgaacaggttatttatcagggtgcaacctatcaggcactgcgtagcaccgaaaatgaagcaccggatacctatctgggtgatgcatggcagcaggttaatgcacagaatgttaccagcagcaccaccccgacctatccggcatatcagagcagcgcagtttatgttggtggtgatcatgttagctataacagccagatctataaagcaaaatggtggacccagggtgaagctccggatgcaacaccgggtacaggtgtttgggaatgggttagcgcagataataatcctgatccgggtcctggtccggatccagatccgacaccggaaccgactccggcaaatggtattattggtcagaacccggatggtagctatattatgagcaaaacatatctggatgcacgtgaagcagaactgaccagcagtccggaatttgcagcagttctggcaagcattagcacccgtgataatgcagttgttgaagcagttgttccgggtctgagcaccaatccggataatgttaaacgtgttgaagccctgattagcgaacagaaatgggatgcactgtttccggaacgtaatgttgtttatacctatagcaacttcctgaaagccgtggcaaaattcaaaggtttttgtgccacctataccgatgaacgtgcagcacagagtgatgcaatttgtgcaaaaagcctggcagttatgtttgcccattttacccaagaaaccggtgcacataatccgcatagcccgtatgaagaatggcgtcagggtctgttttttgttcgtgaagccggttgtagcgaagaagcaagcagctgcggttataatagcgaatgtgcagcaagcaattggcagaccgaacagtggccgtgtggtacaaatcctgatggtagttatgtgaaatactttggtcgtggtgcaaaacagctgagctatcattataactatggtccgtttagcgacttcatcttcaatgatgttaatgtgctgctgcaggaccctgatcgtgttgcagatagctggctgaatctggcaagtgcagtttttttctttgtttatccgcagcctccgaaaccgagcatgctgcatgttattgatggcacctggcagccgaccgcagcagatattgcagaaaaacgcgttccgggttttggtgttaccaccatgattattaacggtggtattgaatgtaccctggataccgaaaaaccgcagagcgttaatcgcattaaatactatcgtggtcatgcagccgcactgggtgttgcaattccggcagatgaacagctgggttgtgcaggtatgaaagcattcaaaaaaaccggtgatagcacctttggtctgtattgggaaaatgattggagctattatcctgataatccgggtggcagcagctttgcatgtcgtattgaaagcggttatcagaccgcacataccaccctgaaaaaaggtgattatgccaaatgcattcagaaatattacggcgtgaccatcgaa

SEQ ID NO: 10 - DNA, 2478 bp, Photobacterium orarium
  Chitinase 3
atggccttagcagcaccgggtacaccgcagctggcatggatggaaaccgattatgcaattgttgaagttgatcaggcagcaaccgcatataaagatctggttaccgttaaaaatgcagccgatgttccggttgcatggcagcgttatagcggtgaaaccgcagatcattggaaagttaaactgaatggtaacgtggcctttgaagaaagcattaatccggcaagcagcggtgcaggtagcaccacactgagcattacccaaggtggtcagtatgcactgaccgttgaactgtgtagcggtagcggtgccgaagaagcatgtagcagcagtccggcaaccaatattgttgttgcagataccgatggtagccatctggatccgctgccgatgaatgttgatccgaataatggcacctatagcacaccggcagatacagttgttggtgcatattttgttgaatggggtgtgtttggtcgcaaatttaccgttgataaaattccggcacagaatctgacccatattctgtatggttttattccggtttgtggtccgaatgaaagcctgggtgaaattgaaagcggtaatagcctggcagcactgaatcgtgcatgtgccggtacaccggattttgaagttgcaattcatgatccgtgggcagcagttcagatgccgcagccgcagagcggtcatacctatagtaccccgtataaaggtaattatggtcagctgatggcactgaaacagcgttatccggatctgaaagttattccgagcattggtggttggaccctgagcgatccgttttttagctttaccgataaaagcaaacgtgatgtttttgttgccagcgtgaaaaaattcctgcagacctggaaatttttcgatggcgttgatatcgattgggaatttcctggtggtaaaggtgcaaataccacactgggtgatccggttaatgatggtccggcatatgttgcactgatgcgtgaactgcgtaccatgctggatggtctggaagcagaaaccggtcgtcagtttgaactgaccagcgcaattggtgttggttatgacaaaattgatgtggtgaattatgccgaagcaagccagtatatggactacatctttaacatgagctacgacttttatggcggttggagcaatgttaccggtcatcagaccgcactgaattgtggttcacatctgacagcagatcagtgtaatggtacaggtgttgatgaaaatggtgaaccgcgtcagggtcctgcatataccaccgcacatggtgttgaacgtctgctggcacagggtgttccggcaaataaactggttgtgggtgcagcaatgtatggtcgcggttggaccggtgttacccaggcaagcatgaccgatccgagcaatccgatgacaggcgttggtaatggtgcagttgccggtagctgggaagccggtgttattgattacaaagatgttgtgacccgctatgaaaacaaagcgggtgttgttctgggttatgatgaacaggccgaagcaccgtgggcatatgatccgtcaaatggtgatctggtgacctatgatagtccgcgtagcattatggcaaaaggccagtatgttcgtgatctgggtttagcaggtctgtttgcatgggaaattgatgcagataatggcgatattctgaacgcaatgcaagaaagtctggcaggcgcaccggcaggtaatcgtgcaccggttgcgcgtgcgggtagcgatcagcaggttaataccgcagcaacagttaccctgaatggttcaagcagtaccgatagtgatggtcagattgcaggttatcagtgggttcagaccagcggtccggcactgacgctgaatggtgccaataccgcaagcgccaccattagcgttccggatgttaccgtggatacccagtatgtgtttaccctgaccgtgaccgataatgaaggtgcaaccgcaaccgatagcattaccgttaccgcaaaagctccgggtgcacagaataccgcaccggtggccagtctgaccggtccgggtacagcaaatgcagatgatgttattaccctggatgcaagccagagcaccgatgcagatggtgataccctgacatatgattggaccgttccggcaggcgttaatgcagttattaatggtagcagcctgagttttaccgcagatagctatacaaccgataccgcactgtcatttagcgttagcgtttcagatggcaccgcaagtgatgcagcaagcctgaccgttacaattgcaaaagatgaaagcggcacaggtggtgaaggtgattttcctgcctatgttgaaggcaccgcatatcaggcaggcgatcaggttagcaatggtggtgttaattttgagtgtaaaccgtatccgtatagcggttggtgtagtggtgcagcatgggcctatgaaccaggtgttggtgtttattggcaggatgcatggacccagctgatcgaa

SEQ ID NO: 11 - DNA, 2331 bp, Photobacterium orarium
  Chitinase 4
atggcccaggcagcagcaaattgtcgtccggatggtctgtatcagacaccgggtgttaccgttccgtattgtaccgtttatgaccaggatggtcgtgaaaaaatgggtgttgatcatccgcgtcgtgtgattggttatttcaccagctggcgtagcggtaatgatccgcagagcagctatctggttaatgatattccgtgggatagtctgacccatatcaattatgcatttgtgagcattggcagtgatggcaaagtgaatattggcaatgttaacaatccggataatccggcagttggtaaagaatggcctggtgttgaaattgatccggcactgggttttaaaggtcattttggtgcactggcaaccgcaaaagcacagcatggtgttaaaaccctgattagcattggtggttgggcagaaaccggtggccattttgatgataatggtaatcgtgttgcagatggtggcttttataccatgacaaccaatgccgatggtagcattaatcatcagggtattcagacctttgcagatagcgcagttgcaatgatgcgtcagtataaattcgatggcctggatatcgattatgaatatccgaccagcatgcaaggtgcaggtaatccggatgattttgcatatagtgatgcaatgcgtccgcatctgatgaaaagctatcatgaactgatgaaagtgctgcgcgaaaaactggatcaggcaagcgcacaggatggacatcattatatgctgaccattgcagcaccgagcagcggttatctgctgcgtggtatggaaaccatgagcgttaccaaatatctggactatgtgaacatcatgagctatgatctgcatggtgcatggaatgattatgttggtcataatgcaagcctgtttgatagcggtctggatgcagaactggaagcaggtaatgtttatggcaccgcacagtacaaaaaaaccggctatctgaatacagattgggcctatcattattttcgtggtagcatgcctgcaggtcgtattaacattggtgtgccgtattacacccgtggttggcagggtgtgaccggtggcaccaatggtctgtggggtaaagcagcactgccgaatcagagcgattgtccgaccggtacaggtgcaggcaccagcaattgcggttatggtgcaattggtattgataacatgtggcacgataaagatgccaatggtaatgaaatgggtgccggtagcaatccgatgtggcatgcaatgaatctggcaaatggtgtttatggtagctataccgcagcatatggtctggatccggttaacgatccgcaggatgcactgaccggtacgtatacccgtcattatgacagcgttagcgttgcaccgtggctgtggaatgcagataaaaaagtttttctgagcaccgaggataaagagagcattaataccaaagccgattacgtgattgaacaaggtattggtggtatcatgttttgggaattagccggtgattatagctgctacaatctggatgcgaatggcaatcgtaccaccgttgatccgaccgaaaatgcatgtaaaaccggtaatggtgaatatcatatgggtgacaccatgaccaaagccatccataacaaatttgcaacagcaaccccgtatggtaataccctggcagaaggtgcaattccgacggaagcagttaatattgatgttagcgttaccggctttaaagtgggcgatcagaattatccgctgaatccgaccatgacactgaccaataaaacgggtcagacactgcctggtggcaccgaatttcagtttgatattcctaccagcacaccggataatatgagcgatcagagcggtgcaaatctgcaggttattagcagcggtcatacccgtggtgacaatattggtggactggatggcaattttcatcgtgtggcatttaccctgccgagctggaaacagctgggtaataatgaatcatttgaactgaccctgaactactttctgccgattagcggtccggcaaattatagcgttcgtgttaataatgcagattatgccctggcatttgaacagccggatctgcctattgcagatctgagcaatggtggtggtgataacggtggcggtgatggtaacaatggtgattgtgttaccaccggtgttaatacctatccgaattggcctcagaccgattgggcaggcaatccgagccatgcaggtacgggtgataaaatcatttataacggtgcagtgtatcaggcaaaatggtggaccaatagcgttccgggtagtgatggtagctgggataccgtttgcaccgttcagatcgaa

SEQ ID NO: 12 - DNA, 1782 bp, Photobacterium orarium
  Chitinase 5
atggccatgggttggagccagattaacattaatggtgttcagaaatgggaagaggcctatagcctgaatgttgatgcagcaaatgaaatcattaccattggtgcagcagataccgcaggcgcactgtatgcaagccagagcctgctgcagctggttgatggtaataaagttccggaagttcagattaccgatgcaccgcgttttgcatatcgtggttttagcgttgatgccgttcgtaattttcgtaccaaagatgcaattattcagctgctggatcagatggcagcattcaaactgaataaactgcatctgcgtctggcagatgatgaaggttggcgtattgaaattgcaggtctgccggaactgaccgatgttggtgcaacccgttgtcatgatccggaagaaaaagcatgtattctgccgtttttaggtgcaggtccggatggtagtccggaaagcaatggttattataccgcaaccgattatcaggatattctgagccatgcagcagcactgaatattgaagttgttccggaaattgatattccgggtcatgcacatgcagcaattaaagccatggaagcacgttatgatcattatgcagcacagggtaatatggcagaagcaaacaaatatctgctgaccgattttaatgataccacgcagtatctgagcgttcagatgtttaccgataatgcgattaatgtgtgtatggaaagcagctacaattttgtggatgcagttgttagcggtctggttagcctgcatcagggtgttcagccgctgaaaacctttcattttggtggtgatgaaattgccggtgcctggattaatagtccggcatgtcaggattttattgccaataacaccgatggtgtgcatagcgttagcgatctgagccgttattttgttgaacgtattagcgttatcaccgccaattatggtctggatatggcaggttgggaagatggtctgatgcatgatggtcaggtttatccgcgtagccagatggcaaataatcagctgtggggtaatgcctggcagaatatttgggaatggggtgttgcagatcgtgcatataatctggccaacaacgattataaagtggtgtataatcatgcgacccacctgtattttgatcatccgtatgaaccggatccgaatgaacgcggttattattgggctccgcgttttacagatacccgtaaaacctttggttttatgccggatgatgtttttgccaatgcagattttacccgtgcgggtgcaccgattaccaaagcagaagttgttgcatcagccggtgttaaaaaactgctgaaaccggaaaatgttctgggtctgcagggtagcctgtgggcagaagcggtgcgtaccgaagatcagtttgagggtatgatttttccgcgtgttttaggtctggccgaacgtgcatggcataccgccacctgggaagcaaatgataatgcaggtattgcactggatgaaaccggtcgtaatgccgattataaccattttgcaaatctgctgggtcagaaagttctgccgaaactggaacaggcaggcattgcatttaatctgccggttccaggtggtgtgattgaaaatggtgtgctgcaggcaaatagcacctttccgggtctgaccattgaatatagcaccgatcagggcaccagctggcagagctatgatcatctgaatccgcctgcagttgcagcgggtgtgcagctgcgtaccgtgagcggtcagcgtaccagccgtgttaccaccgttaatatcgaa

SEQ ID NO: 13 - DNA, 46 bp, Artificial Sequence
  Figure 10 Sequence 1
  unsure 7..12
  unsure 35..40
ggtctcnnnnnnatatatatatatatatatatatnnnnnngagacc

SEQ ID NO: 14 - DNA, 36 bp, Artificial Sequence
  Figure 10 Sequence 2
  unsure 7..12
  unsure 25..30
ggtctcnnnnnngcgcgcgcgcgcnnnnnngagacc

SEQ ID NO: 15 - DNA, 28 bp, Artificial Sequence
  Figure 10 Sequence 3
  unsure 1..5
  unsure 28
nnnnnatatatatatatatatatatatn

SEQ ID NO: 16 - DNA, 18 bp, Artificial Sequence
  Figure 10 Sequence 4
  unsure 1..5
  unsure 18
nnnnngcgcgcgcgcgcn

SEQ ID NO: 17 - DNA, 46 bp, Artificial Sequence
  Figure 10 Sequence 5
  unsure 1..5
  unsure 18..23
  unsure 46
nnnnngcgcgcgcgcgcnnnnnnatatatatatatatatatatatn

SEQ ID NO: 18 - DNA, 24 bp, Artificial Sequence
  T7F Primer
aaattaatacgactcactataggg

SEQ ID NO: 19 - DNA, 21 bp, Artificial Sequence
  T7R Primer
atgctagttattgctcagcgg